(19) United States
(12) Patent Application Publication
(10) Pub. No.: US 2009/0156537 A1
(43) Pub. Date: Jun. 18, 2009

(54) COMPOSITIONS AND METHODS FOR DIAGNOSING AND TREATING ASTHMA OR OTHER ALLERGIC OR INFLAMMATORY DISEASES

(75) Inventors: Michael R. Bowman, Westwood, MA (US); Maximilliam T. Follettie, Belmont, MA (US); Hang Chen, Framingham, MA (US); Cara Williams, Methuen, MA (US); Aaron Winkler, Arlington, MA (US); Debra Ellis, Lowell, MA (US); Wei Liu, Lexington, MA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/257,852

(22) Filed: Oct. 24, 2008

Related U.S. Application Data
(62) Division of application No. 10/792,280, filed on Mar. 4, 2004, now abandoned.
(60) Provisional application No. 60/451,396, filed on Mar. 4, 2003.
(60) Provisional application No. 60/475,870, filed on Jun. 5, 2003.

(51) Int. Cl. A61K 31/7105; A61P 11/06; A61P 11/00; A61K 31/198

(52) U.S. Cl. 514/44; 514/564

(57) ABSTRACT

The present invention provides compositions and methods useful for detecting or treating asthma or other allergic or inflammatory diseases. In one aspect, the methods of the present invention include inhibiting the activity or expression of a component of an arginine metabolic pathway in tissues affected by asthma or other allergic or inflammatory diseases. In many embodiments, the component being inhibited is a cationic amino acid transporter, an arginase, or a component downstream of the arginase. In many other embodiments, the activity or expression of the component is inhibited by an agent that binds to the component. In still many other embodiments, the activity or expression of the component is inhibited by an agent that binds a polynucleotide encoding the component.

### TECHNICAL FIELD

The present invention relates to compositions and methods useful for the diagnosis or treatment of asthma or other allergic or inflammatory diseases.

### BACKGROUND

Asthma is a chronic inflammatory disease of the airways that is characterized by recurrent episodes of reversible airway obstruction and airway hyperresponsiveness (AHR). Typical clinical manifestations include shortness of breath, wheezing, coughing and chest tightness that can become life threatening or fatal. While existing therapies focus on reducing the symptomatic bronchospasm and pulmonary inflammation, there is a growing awareness of the role of long-term airway remodeling in accelerated lung deterioration in asthmatics. Airway remodeling refers to a number of pathological features including epithelial smooth muscle and myofibroblast hyperplasia and/or metaplasia, subepithelial fibrosis and matrix deposition. The processes collectively result in up to about 300% thickening of the airway in cases of fatal asthma. Despite the considerable progress that has been made in elucidating the pathophysiology of asthma, the prevalence, morbidity, and mortality of the disease has increased during the past two decades. In 1995, in the United States alone, nearly 1.8 million emergency room visits, 466,000 hospitalizations and 5,429 deaths were directly attributed to asthma.

It is generally accepted that allergic asthma is initiated by an inappropriate inflammatory reaction to airborne allergens. The lungs of asthmatics demonstrate an intense infiltration of lymphocytes, mast cells and eosinophils. A large body of evidence has demonstrated this immune response is driven by CD4⁺ T-cells expressing a T_{H}2 cytokine profile. One murine model of asthma involves sensitization of the animal to ovalbumin (OVA) followed by intratracheal delivery of the OVA challenge. This procedure generates a T_{H}2 immune reaction in the mouse lung and mimics four major pathophysiological responses seen in human asthma, including upregulated serum IgE (atopy), eosinophilia, excessive mucus secretion, and AHR. The cytokine IL-13, expressed by basophils, mast cells, activated T cells and NK cells, plays a central role in the inflammatory response to OVA in mouse lungs. Direct lung instillation of murine IL-13 elicits all four of the asthma-related pathologies and, conversely, the presence of a soluble IL-13 antagonist (sIL-13Rα2-Fc) completely blocked both the OVA-challenge induced goblet cell mucus synthesis and the AHR to acetylcholine. Thus, IL-13 mediated signaling is sufficient to elicit all four asthma-related pathophysiological phenotypes and is required for the hypersecretion of mucous and induced AHR in the mouse model.

Biologically active IL-13 binds specifically to a low-affinity binding chain IL-13Rα1 and to a high-affinity multimeric complex composed of IL-13Rα1 and IL-4R, a shared component of IL-4 signaling complex. The high-affinity complex is expressed in a wide variety of cell types including monocyte-macrophage populations, basophils, eosinophils, mast cells, endothelial cells, fibroblasts, airway smooth muscle and airway epithelial cells. IL-13 mediated ligation of the functional receptor complex results in the phosphorylation dependent activation of JAK1 and JAK or Tyk-2 kinases and IRS1/2 proteins. Activation of the IL-13 pathway cascade triggers the recruitment, phosphorylation and ultimate nuclear translocation of the transcriptional activator Stat6. A number of physiological studies demonstrate the inability of pulmonary OVA-challenge to elicit major pathology related phenotypes including eosinophil infiltration, mucus hypersecretion and airway hyperreactivity in mice homozygous for the Stat6^{-/-} null allele. Recent genetic studies have demonstrated a linkage between specific human alleles of IL-13 and its signaling components with asthma and atopy, demonstrating the critical role of this pathway in the human disease.

IL-13 also binds to an additional receptor chain, IL-13Rα2, expressed in both human and mouse with as yet undefined biological function. The murine IL-13Rα2 binds IL-13 with approximately 100-fold greater affinity (Kd of 0.5 to 1.2 nM) relative to IL-13Rα1, allowing the construction of a potent soluble IL-13 antagonist, sIL-13Rα2-Fc. The sIL-13Rα2-Fc has been used as an antagonist in a variety of disease models to demonstrate the role of IL-13 in Schistosomiasis induced liver fibrosis and granuloma formation, tumor immune surveillance, as well as in the OVA-challenge asthma model.

Chronic obstructive pulmonary disease (COPD) is an umbrella term used to describe airflow obstruction that is associated mainly with emphysema and chronic bronchitis. Emphysema causes irreversible lung damage by weakening and breaking the air sacs within the lungs. As a result, elasticity of the lung tissue is lost, causing airways to collapse and obstruction of airflow to occur. Chronic bronchitis is an inflammatory disease that begins in the smaller airways within the lungs and gradually advances to larger airways. It increases mucus in the airways and bacterial infections in the bronchial tubes, which, in turn, impedes airflow.

COPD affects tens of millions of Americans and is a serious health problem in the U.S. A 1998 prevalence survey suggest that three million Americans have been diagnosed with emphysema and nine million are affected by chronic bronchitis. COPD is the fourth leading cause of death in the U.S. in 1998 and accounted for 112,584 deaths in 1998. COPD also accounted for an estimated 668,362 hospital discharges in 1998.

Current therapy for asthma and COPD includes use of bronchodilators, corticosteroids, and leukotriene inhibitors. The treatments share the same therapeutic goal of bronchodilation, reducing inflammation, and facilitating expectoration. Many of such treatments, however, include undesired side effects and lose effectiveness after being used for a period of time. Additionally, only limited agents for therapeutic intervention are available for decreasing the airway remodeling process that occurs in asthmatics. Therefore, there remains a need for an increased molecular understanding of asthma and COPD, and a need for the identification of novel therapeutic strategies to combat these complex diseases.

### SUMMARY OF THE INVENTION

The present invention identifies numerous genes that are differentially expressed in asthmatic lung tissues as compared to non-asthmatic lung tissues. The genes thus identified include members of arginine metabolic pathways, such as cationic amino acid transporter 2 gene (CAT2) and arginase type I gene (ARG1). These genes are potential drug targets for treating asthma or other allergic or inflammatory diseases.

In one aspect, the present invention provides methods for treating allergic or inflammatory diseases. The methods include administering a therapeutically effective amount of an agent to a mammal which has an allergic or inflammatory disease, where the agent inhibits the activity or expression of a component of the arginine metabolic pathway in tissues affected by the disease. The component being inhibited is not a nitric oxide synthase (NOS). In many embodiments, the component being inhibited is an arginase (e.g., arginase type I) or a protein downstream thereof. Examples of the downstream proteins include, but are not limited to, ornithine decarboxylase, ornithine aminotransferase, ornithine transcarbamylase, spermidine synthase, and spermine synthase. In one instance, S-adenosylmethionine decarboxylase, which is involved in the biosynthesis of polyamines, may also be inhibited. In another embodiment, the component being inhibited is a cationic amino acid transporter (e.g., cationic amino acid transporter 2).

Allergic or inflammatory diseases amenable to the present invention include, but are not limited to, asthma, airway hyperresponsiveness, chronic airway remodeling, chronic obstructive pulmonary disease (COPD), and arthritis. Other diseases associated with dysfunctions or abnormalities in arginine metabolism can also be treated by the present invention. In many embodiments, the allergic or inflammatory diseases are respiratory diseases. Administration of a therapeutic agent of the present invention inhibits the activity or expression of a component of an arginine metabolic pathway in lung tissues, thereby ameliorating or eliminating syndromes associated with the diseases.

Therapeutic agents suitable for the present invention include, but are not limited to, polynucleotides capable of inhibiting the expression of the target component by RNA interference or an antisense mechanism, antibodies reactive with the target component, inhibitors of a biological function of the target component, or other modulators that can bind to the target component or the polynucleotides encoding the same (e.g., mRNA or genomic sequences, including the 3′ or 5′ untranslated regulatory sequences). In many embodiments, the activity or expression is inhibited at the transcriptional, post-transcriptional, translational, or post-translational level. In many other embodiments, the inhibitory agents can decrease the activity or expression of the target component by at least 10%, 20%, 30%, 40%, 50%, 60%, 70%, 80%, 90%, 95% or more as compared to the original activity or expression level.

In one embodiment, the therapeutic agents of the present invention encode or comprise siRNA sequences that are directed to CAT2, ARG1, or other genes that encode components downstream of arginase. In another embodiment, the therapeutic agents are expressed from gene therapy vectors. In many instances, the gene therapy vectors are under control of a tissue- or cell-specific promoter. In one example, the promoter is lung specific. Examples of lung-specific promoters include, but are not limited to, the lung epithelial cell-specific surfactant protein B gene promoter and the Clara cell-specific promoter CC10. In another example, the promoter is monocyte or macrophage specific. Examples of macrophage-specific promoters include, but are not limited to, the proximal promoter of the human acetyl-LDL receptor (SRA) gene and those described in Ross et al., J. Biol. Chem., 273:6662-6669, 1998).

In yet another embodiment, the therapeutic agents of the present invention are selected from lysine, poly-L-lysine, poly-L-arginine, or other cationic polypeptides that can inhibit cationic amino acid transporters. In a further embodiment, the therapeutic agent is α-difluoromethylomithine which inhibits the function of ornithine decarboxylase. In still another embodiment, the therapeutic agent is an IL-13 antagonist or an antagonistic anti-IL-13 antibody. In one example, the therapeutic agent is a soluble IL-13 receptor.

The therapeutic agents of the present invention can be formulated to be compatible with their intended routes of administration. Examples of routes of administration include, but are not limited to, parenteral, enteral, and topical administration. For instance, a therapeutic agent of the present invention can be administered via intracutaneous, epicutaneous, inhalative, oral, rectal, intravenous, intraarterial, intramuscular, subcutaneous, intradermal, transdermal, transmucosal, or other suitable routes. In one embodiment, the therapeutic agent is administered via inhalation. For instance, the therapeutic agent can be delivered in the form of an aerosol spray from a pressured container or dispenser which contains a suitable propellant (e.g., carbon dioxide) or nebulizer.

In one embodiment, the mammal being treated is a human who has asthma or another allergic or inflammatory disease.

In another aspect, the present invention provides methods useful for identifying or evaluating drugs for the treatment of asthma or other allergic or inflammatory diseases. The methods include contacting a candidate molecule with a tissue affected by asthma or another allergic or inflammatory disease, and determining if the candidate molecule can ameliorate or eliminate a disease syndrome or phenotype in the tissue. The candidate molecule inhibits the activity or expression of a non-NOS component of an arginine metabolic pathway in the tissue. Exemplary non-NOS components include, but are not limited to, arginase or cationic amino acid transporter. Tissues suitable for use in the present invention include, but are not limited to, tissues/cells in animal models of the disease, tissues/cells isolated from animal models of the disease, or cell cultures that mimic certain aspects (e.g., expression profiles) of disease-affected tissues/cells. In one example, the therapeutic effect of a candidate molecule is assessed by a human clinical trial.

In one embodiment, the candidate molecule is selected or generated based on a structure-based rational drug design. Molecules capable of interacting with a non-NOS component of an arginine metabolic pathway are identified. These molecules are then brought into contact with tissues affected by asthma or other allergic or inflammatory diseases to determine if they can ameliorate or eliminate disease syndromes or phenotypes.

In another embodiment, high throughput screening methods or compound libraries are used to identify drug candidates.

The present invention also features methods useful for detecting, diagnosing, or monitoring asthma or other allergic or inflammatory diseases. The methods include detecting an expression profile of at least one gene in a biological sample of a ma mal, and comparing the expression profile to a reference expression profile of the gene to determine if the mammal has or is at risk for an allergic or inflammatory disease. In many cases, the gene encodes a non-NOS component of an arginine metabolic pathway.

In one embodiment, the allergic or inflammatory disease is asthma or COPD. The biological sample can be a lung sample. Mucus, blood, or other types of samples can also be used. In another embodiment, the reference expression profile is an average expression profile of the arginine metabolic gene in disease-affected tissues. The reference expression profile can also be an expression profile of the arginine metabolic gene in disease-affected tissues. In yet another embodiment, the arginine metabolic gene is selected from ARG1 or CAT2. The materials used for the detection or diagnosis of asthma or other allergic or inflammatory diseases can be included in a kit.

In yet another aspect, the present invention provides pharmaceutical compositions that are useful for treating asthma or other allergic or inflammatory diseases. The pharmaceutical compositions include a pharmaceutically-acceptable carrier and a therapeutically effective amount of an agent which is capable of inhibiting an activity or expression of a non-NOS component of an arginine metabolic pathway. In many embodiments, the agent can bind to the non-NOS component, or a polynucleotide encoding the same. In one example, the non-NOS component is encoded by ARG1 or CAT2.

Other objects, features and advantages of the present invention will become apparent from the following detailed description. The detailed description and specific examples, while indicating preferred embodiments, are given for illustration only since various changes and modifications within the scope of the invention will become apparent to those skilled in the art from this detailed description. Further, the examples demonstrate the principle of the invention and should not be expected to specifically illustrate the application of this invention to all the examples of infections where it obviously will be useful to those skilled in the prior art.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The drawing is provided for illustration, not limitation.

FIG. 1 is a graphical representation showing that CAT2 and arginase type I (ARG1) are coinduced in Balb/c mice by both allergen (OVA) and recombinant murine IL-13 (IL-13). Briefly, Balb/c mice were sensitized to OVA by intraperitoneal injection on Day 0, challenged by intratracheal (IT) injection of either vehicle (phosphate buffered saline (PBS)) or OVA on Days 14 and 25, and the lungs harvested at Day 28. Additionally, naïve Balb/c mice were treated IT with either PBS or IL-13 for 3 consecutive days and the lungs harvested at 72 hours. Total lung RNA was isolated and analyzed for mRNA expression using GeneChip technology (Affymetrix) as described in Example 1. The mRNA frequency is expressed as parts per million.

FIG. 2 is a graphical representation showing that ARG1 expression is induced in Balb/c mice by both allergen (OVA) and recombinant murine IL-13 (IL-13). Briefly, Balb/c mice were sensitized to OVA by intraperitoneal injection on Day 0, challenged by intratracheal (IT) injection of either vehicle (phosphate buffered saline (PBS)) or OVA on Days 14 and 25, and the lungs harvested at Day 28. Additionally, naïve Balb/c mice were treated IT with either PBS or IL-13 for 3 consecutive days and the lungs harvested at 72 hours. Total lung RNA was isolated and analyzed for mRNA expression using GeneChip technology (Affymetrix) as described in Example 1. The mRNA frequency is expressed as parts per million.

FIG. 3 is a graphical representation showing that ARG1 gene is induced by OVA or adenovirus-mediated expression of IL-13 in Balb/c mice. Briefly, Balb/c mice were intratracheally inoculated with recombinant adenovirus expression murine IL-13 or murine secreted alkaline phosphatase (SEAP) on Day 0, and the lungs harvested at Day 3. Control mice were treated with PBS, OVA, or IL-13 as described in FIG. 1. The mRNA frequency is expressed as parts per million.

FIG. 4 is a graphical representation showing that ARG1 gene is induced by adenovirus-mediated expression of IL-13 in C57bl/6 mice. Briefly, C57bl/6 mice were intratracheally inoculated with recombinant adenovirus expression murine IL-13 or murine secreted alkaline phosphatase (SEAP) on Day 0, and the lungs harvested at Day 3. The mRNA frequency is expressed as parts per million.

FIG. 5 is a graphical representation showing that arginine uptake is optimally induced by LPS IL-13 in the murine macrophage cell line RAW264.7. Briefly, RAW264.7 macrophages were induced to express various levels of CAT2 by treatment for 24 hours with LPS and/or IL-13. Arginine transport in the presence or absence of competing L-Lysine was evaluated over a three-minute period in a final arginine concentration of 400 μM as described in Example 3. Specific arginine uptake (CPM/mg protein lysate) is expressed as a percentage of that measured in un-stimulated cells.

FIG. 6 is a graphical representation showing that CAT2 and ARG1 are coinduced by lipopolysaccharide (LPS)/IL-13 in the murine macrophage cell line RAW264.7. Briefly, RAW264.7 cells were treated with 10 ng/ml IL-13 and/or 1 μg/ml LPS. Total RNA isolated from cells 24 hours after the treatment was analyzed for ARG1 and CAT isoform-specific mRNA expression using TaqMan real-time quantitative RT-PCR as described in Example 2. Glucose-6-phosphate dehydrogenase (GAPDH)-normalized mRNA frequency was estimated from the threshold cycle numbers using the methods of Fink et al. (Fink et al., Nat. Medicine, 4:1329-1333, 1998) and expressed as copies/copy of GAPDH.

FIG. 7 is a graphical representation showing that arginine uptake is inhibited by 20 mM lysine in LPS/L-13 treated RAW264.7 murine macrophage cells. Briefly, RAW264.7 cells were exposed to media alone (Control) or CAT2-inducing conditions (LPS/IL-13) for 24 hours and were then evaluated for arginine transport over a three-minute period in a final arginine concentration of 100 μM. The addition of the competitive CAT inhibitor 20 mM lysine to the transport buffer abolished all saturable arginine transport in Control and LPS/IL-13 treated cells.

FIG. 8 is a graphical representation showing that urea production in the murine macrophage cell line RAW264.7 is inhibited by 20 mM lysine. Briefly, RAW264.7 macrophages exposed to media alone (Control) or CAT2-inducing conditions (LPS/IL-13) for 24 hours were then equilibrated for 2 hours in Arginine Transport Buffer. After a 24 hour incubation in the presence or absence of competing L-lysine in Arginine Transport Buffer containing a final arginine concentration of 400 μM, urea production was evaluated as described in Example 4. Urea production is expressed as μg of urea in the supernatant/mg of cell lysate protein.

FIG. 9 is a graphical representation showing that carbachol-induced rat tracheal contraction is inhibited by 100 mM lysine. Briefly, rat tracheal explants were preincubated for 15 to 20 hours with either media or media containing 100 mM L-lysine. The trachea were then washed and contraction measured in a Krebs-Henseleit solution in the presence or absence of 100 mM L-lysine. Tensions were calculated as mg of tension/mg of trachea and expressed as mean and standard error of % of maximal contraction (i.e. the contraction evoked by 10⁻⁵ M carbachol in the absence of lysine).

FIG. 10 is a graphical representation showing that induction of ARG1 expression requires IL-4 receptor. Briefly, IL-4 receptor knockout mice (IL4R−/−) and IL-4 knockout mice (IL4−/−) sensitized to OVA, or treated with PBS or IL-13 as described in FIG. 1. Total lung RNA was isolated and analyzed for mRNA expression using GeneChip technology (Affymetrix) as described in Example 1. The mRNA frequency is expressed as parts per million.

FIG. 11 compares tracheal contraction in CAT-2 knockout mice to that in wild-type mice. CATS2-KO denotes CAT2 knockout mice.

FIG. 12 is a graphical representation showing that ARG1 mRNA expression increases following direct pulmonary instillation of rIL-13 or intratracheal Ovalbumin allergen challenge. Blockade of IL-13 signaling by administration of sIL13Rα2.Fc inhibits 67% of induced Arg1 mRNA expression. Blockade of IL-13 using the soluble receptor sIL13Rα2.Fc inhibits the allergen induced mucus production and airway hyperresponsiveness (AHR).

### DETAILED DESCRIPTION OF THE INVENTION

The present invention relates to compositions and methods useful for the diagnosis and treatment of asthma or other allergic or inflammatory diseases. In one aspect, the methods of the present invention include inhibiting the activity or expression of a component of an arginine metabolic pathway in tissues affected by asthma or other allergic or inflammatory diseases. In many embodiments, the component being inhibited is a cationic amino acid transporter, an arginase, or a component downstream of the arginase. Inhibition of the activity or expression of these components reduces or eliminates the disease syndrome or phenotype in the affected tissues. The present invention also provides methods for identifying therapeutic agents for treating asthma or other allergic or inflammatory diseases.

Various aspects of the invention are described in further detail in the following subsections. The use of subsections is not meant to limit the invention; subsections may apply to any aspect of the invention. In this application, the use of “or” means “and/or” unless stated otherwise.

### CAT2, ARG1 and Inflammatory Diseases

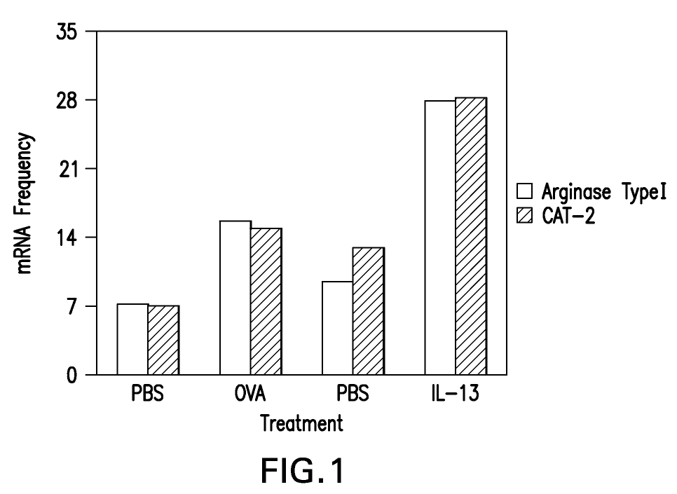

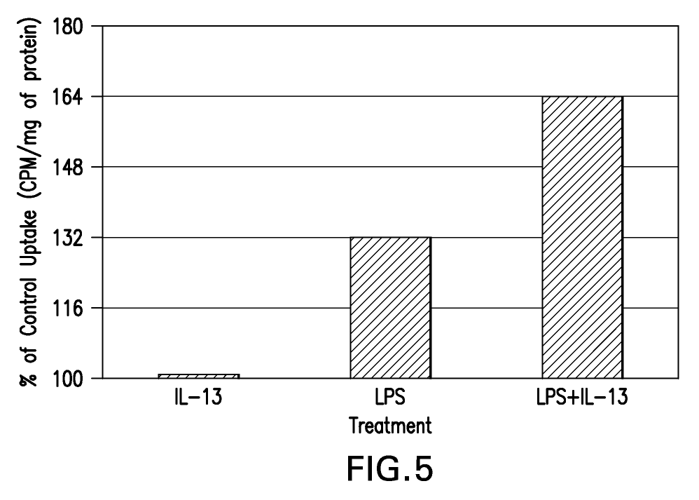

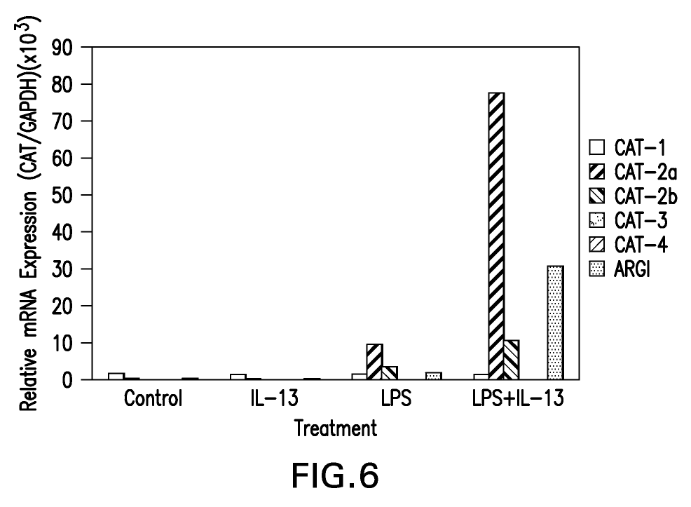

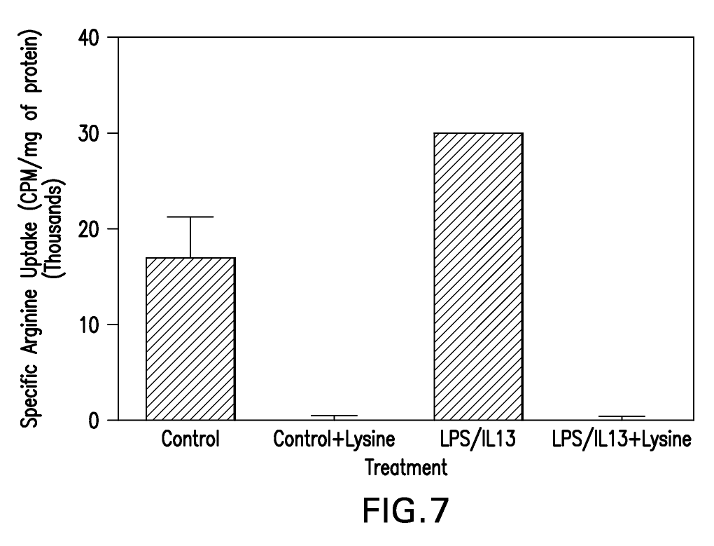

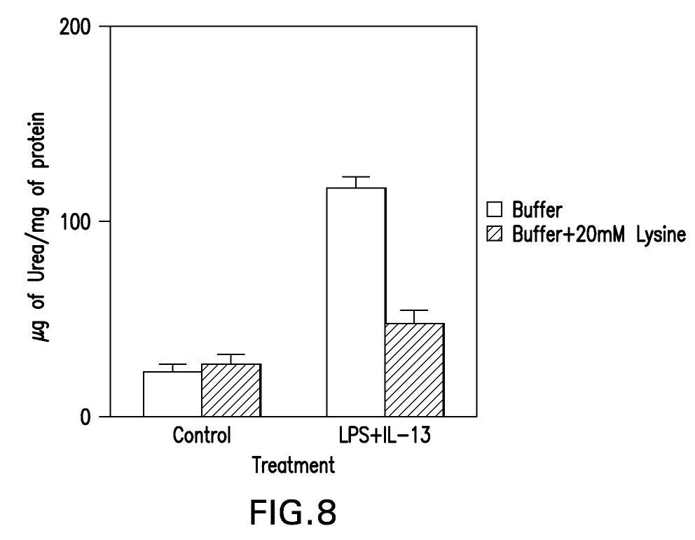

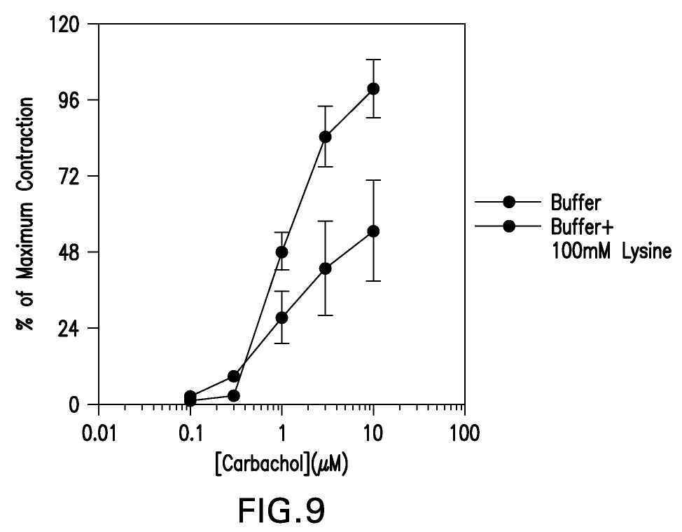

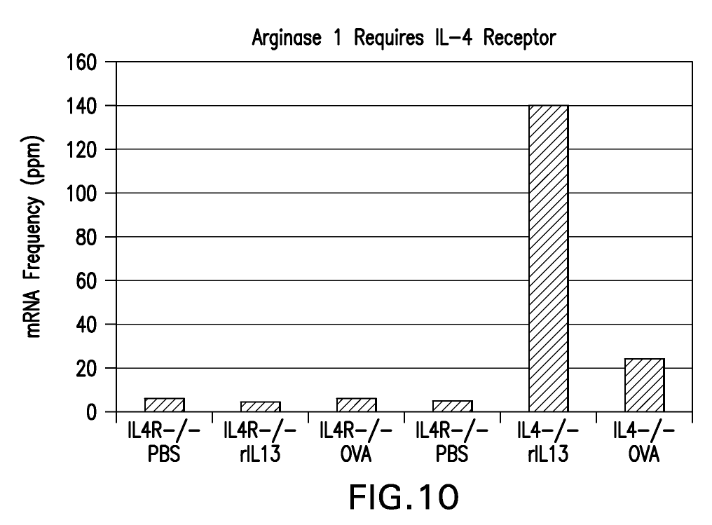

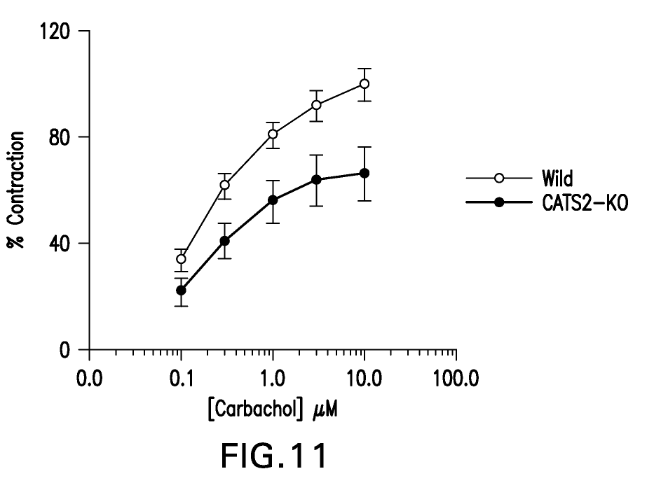

Intratracheal OVA challenge in sensitized mice generates a T_{H}2 immune reaction in the lung that mimics several physiological characteristics of human allergic asthma. Significant evidence has demonstrated the central role of IL-13 mediated signal transduction in this animal disease model. Oligonucleotide arrays were used to profile the transcriptional changes in mouse lung tissue following either intratracheal OVA challenge or direct lung instillation of IL-13. As shown in FIGS. 1-4, mRNA frequencies of CAT2 and/or ARG1 are significantly increased when mice were treated with either OVA or IL-13. Likewise, CAT2 and ARG1 are also co-induced in murine macrophage cells RAW264.7 treated with a combination of lipopolysaccharide (LPS) and IL-13 (FIGS. 5 and 6). The induced CAT2 and ARG1 expression is associated with an increase in arginine transport (FIG. 7) but also an increase in urea production in RAW264.7 cells (FIG. 8), suggesting the activation of the arginase pathway. Further studies revealed that the increase in arginine uptake and urea production can be inhibited using a competitive inhibitor of CAT2, lysine (FIGS. 7 and 8). In addition, carbachol-induced tracheal contraction is also inhibited by lysine (FIG. 9) or a genetic deletion of the CAT2 gene (FIG. 11), further suggesting the involvement of CAT2 in the pathophysiology of inflammatory diseases. Furthermore, the induction of ARG1 expression requires the IL-4 receptor is demonstrated in FIG. 10. Lastly, administration of soluble IL13Rα2.Fc. blocks IL-13 signaling, which, in turn, inhibits ARG1 mRNA expression.

Arginine is a semi-essential amino acid that is metabolized to important regulatory molecules. Arginine is transported into vascular smooth muscle cells (SMC) by the cationic amino acid transporter (CAT) family of proteins where it is metabolized to nitric oxide (NO), polyamines, or proline. Inflammatory mediators, growth factors, and hemodynamic forces stimulate the transport of arginine in vascular SMC by inducing CAT gene expression. Inflammatory cytokines also induce the expression of inducible NO synthase (iNOS) and direct the metabolism of arginine to the antiproliferative gas, NO. In contrast, cyclic mechanical strain blocks both iNOS and ODC activity and stimulates arginase I gene expression, directing the metabolism of arginine to the formation of L-proline and collagen.

In non-hepatic tissue, which is incapable of recycling arginine, the upregulated CAT2 transporter supplies the increased arginase activity with a sufficient supply of substrate, arginine. This increase in arginase activity is part of a biochemical pathway critical for such pathogenic processes as fibrosis, airway hyper-responsiveness, goblet cell hyperplasia, oxidative stress associated apoptosis, and airway inflammation, which are commonly found in inflammatory diseases. Accordingly, inhibition of CAT2's transport of arginine will block the induced non-hepatic arginase pathway while sparing the hepatic urea cycle, which also utilizes arginase but is able to recycle arginine as a substrate.

### The Biochemical and Biological Characteristics of CAT2

The nucleotide and amino acid sequences of human CAT2 (also known as SLC7A2) are set forth in SEQ ID NOS:1 and 2, respectively. The nucleotide and amino acid sequences of murine CAT2 are set forth in SEQ ID NOS:3 and 4, respectively.

Human CAT2 cDNA (SEQ ID NO:1) was isolated from a human intestine cDNA library. The nucleotide sequence of the coding region predicts a 658-amino-acid protein (SEQ ID NO:2) with a calculated molecular weight of 71,669. As 91% of the residues are identical with those of the mouse CAT2, human CAT2 seems to be a human counterpart of the mouse CAT2. In Northern blot analysis, a single (9.0 kb) human CAT2 mRNA transcript was present in various tissues. The highest level of expression was observed in skeletal muscle and the lowest level in the kidney. Hydropathy plots indicated that the translated protein is predicted to have 14 transmembrane domains with three potential N-glycosylation sites. The human CAT2 gene was assigned to human chromosome 8p21.3-p22.

Analysis of the genomic organization revealed that human CAT2 consists of 12 translated exons and most likely of 2 untranslated exons. The CAT2 gene encodes two protein isoforms, CAT2A and CAT2B, that result from mutually exclusive alternate splicing (exon 7 for CAT2A and exon 6 for CAT2B). The human CAT2 gene structure is closely related to the structure of human CAT1, suggesting that they belong to a common gene family.

In mouse, the CAT2 gene is transcribed from five distinct promoters dispersed over a space of 18 kb, which result in several distinct CAT2 mRNA isoforms due to transcriptional initiation at distinct promoters. The isoform adjacent to the most distal promoter is found in all tissues and cell types previously shown to express mouse CAT2, while the other 5′ UTR isoforms are more tissue specific in their expression. Utilization of some or all of five putative promoters was documented in Lymphoma cell clones, liver, and skeletal muscle. TATA-containing and (G+C)-rich TATA-less promoters appear to control mouse CAT2 gene expression. It was suggested that the multiple isoforms of CAT2 mRNA permit flexible transcriptional regulation of this cationic amino acid transporter gene.

Since CAT2 plays an important role in the production of NO, which is a highly reactive free radical that is associated with a variety of diseases including cancer, inhibition of CAT2 has been proposed as a treatment for diseases characterized by undesirable levels of NO. For example, U.S. Pat. No. 5,866,123 to MacLeod describes a method to inhibit CAT2 expression by an antibody raised against the mouse CAT2 protein. International Patent Application WO 00/44766 also describes methods of inhibiting CAT2 expression by both antisense and antibody technology.

### The Biochemical and Biological Characteristics of ARG1

The nucleotide and amino acid sequences of human ARG1 are set forth in SEQ ID NOS:5 and 6, respectively. The nucleotide and amino acid sequences of murine ARG1 are set forth in SEQ ID NOS:7 and 8, respectively.

Arginase catalyzes the hydrolysis of arginine to ornithine and urea. At least two isoforms of mammalian arginase exist (types I and II) which differ in their tissue distribution, subcellular localization, immunologic crossreactivity and physiologic function. ARG1 encodes the type I isoform, which is a cytosolic enzyme and expressed predominantly in the liver as a component of the urea cycle. ARG1 functions as a trimer of three identical subunits. Inherited deficiency of this enzyme results in argininemia, an autosomal recessive disorder characterized by hyperammonemia.

The structure of the trimeric rat ARG1 has been determined at 2.1-A resolution (Kanyo et al., Nature 383:554-55, 1996). A key feature of the structure is a novel S-shaped oligomerization motif at the carboxyl terminus of the protein that mediates approximately 54% of the intermonomer contacts. Arg-308, located within this oligomerization motif, nucleates a series of intramonomer and intermonomer salt links. In contrast to the trimeric wild-type enzyme, the R308A, R308E, and R308K variants of rat ARG1 exist as monomeric species, as determined by gel filtration and analytical ultracentrifugation, indicating that mutation of Arg-308 shifts the equilibrium for trimer dissociation by at least a factor of 105. These monomeric arginase variants are catalytically active, with k_{cat}/Kₘ values that are 13-17% of the value for wild-type enzyme. The rat ARG1 variants are characterized by decreased temperature stability relative to the wild-type enzyme. The crystal structure of the complex between human arginase and a boronic acid analog of L-arginine, 2(S)-amino-6-boronohexanoic acid (ABH), has been determined at 1.7 A resolution (Cox et al., Nat. Struct. Biol. 6: 1043-1047, 1999). Mutational analyses have also revealed that amino acid residues Lys-141, Glu-256, and Gly-235 are critical for the function of human ARG1.

The human ARG1 gene has been cloned and the structure determined. The human ARG1 gene is 11.5 kilobases long and is split into 8 exons. The cap site was determined by nuclease S1 mapping and primer extension. A “TATA box”-like sequence is located 28 bases upstream from the cap site, and a sequence similar to the binding sites of the transcription factor CTF/NF1, a “CAAT box”-binding protein, is located 72 bases upstream. In the 5′ end region, sequences resembling the glucocorticoid responsive elements, the cAMP responsive elements, and the enhancer core sequences are present. The immediately 5′ flanking region of the human ARG1 gene up to position −105 is 84% identical with the corresponding segment of the rat gene. In this region of the human ARG1 gene, one DNase I-protected area and several hypersensitive cleavage sites were detected by footprint analysis. The protected area contains the sequence similar to the binding sites of CTF/NF1 and also overlaps with the sequence resembling the glucocorticoid responsive elements.

A number of assay procedures have been developed to measure arginase activity. For example, Greenberg described an enzymatic assay in Arginase, The Enzymes 4, edited by P. Boyer, H. Lardy, and K. Myrback, Academic Press, NY, 257, 1960. Geyer et al. described an assay for arginase in tissue homogenates (Geyer et al. Anal. Biochem. 39:412, 1971). Nishibe and Makino reported an automated assay method for erythrocyte arginase (Nishibe et al., Anal. Biochem. 43: 357, 1971). A microassay was also described (Hirsch-Kolb et al., Anal. Biochem. 35: 60, 1970). Most methods are based upon the colorimetric determination of the urea nitrogen released during the hydrolysis of arginine.

Over-expression of arginase has been detected in patients with colorectal carcinoma (Porembska et al., Cancer 94: 2930-2934, 2002). Increased arginase activity has been associated with allergen-induced deficiency of cNOS-derived nitric oxide and airway hyperresponsiveness (Meurs et al, Br. J. Pharmacol. 136: 391-398, 2002).

### Inhibition of Arginase Activity

Arginase activity can be inhibited by many amino acids, such as valine, lysine, leucine, isoleucine, proline and threonine, as well as arginine analogues and derivatives such as L-canavame (Can) and L-ornithine (Orn). All these amino acids function as competitive inhibitors. Orinithine and urea, the products of the reaction catalyzed by arginase, also function as competitive inhibitors of arginase. The competitive inhibition by the products ornithine and urea indicates a rapid-equilibrium random mechanism for the enzyme.

Arginase activity is associated with a tightly bound Mn⁺⁺ whose catalytic action may be stimulated by addition of a more loosely bound Mn⁺⁺, to generate a fully activated enzyme form. However, despite this requirement for added divalent cations in the activation of arginase, metal chelators such as EDTA and citrate do not inhibit the enzyme. It thus appears that the metal binding site is not readily accessible to solvent. On the other hand, arginase activity is inhibited by a borate, which behaves as an S-hyperbolic I-hyperbolic non-competitive inhibitor and had no effect on the interaction of the enzyme with the competitive inhibitors L-ornithine (Ki=2+/−0.5 mM), L-lysine (Ki=2.5+/−0.4 mM), and guanidinium chloride (Ki=100+/−10 mM). It has been proposed that borate binds in close proximity to the loosely bound Mn⁺⁺ and interferes with its stimulatory action. It was further suggested that borate inhibition arises from chelation of Mn⁺⁺ in the binuclear Mn⁺⁺ center, thus displacing a metal-bound water molecule that is responsible for nucleophilic attack on the guanidium carbon (Carvajal et al., J. Inorg. Biochem. 77: 163-167, 1999). Other experiments also demonstrate that borate and urea bind in a mutually exclusive manner, while L-ornithine and borate can bind simultaneously to the enzyme.

The inhibitory effects of anions, such as N₃⁻, NO₂³⁻, BO₄³⁻, SCN⁻, CH₃COO⁻, SO₄²⁻, ClO₄⁻, H₂PO₄⁻, CN⁻, I⁻, Br⁻, Cl⁻ and F⁻, on the hydrolysis of L-arginine (L-Arg) by rat liver arginase (RLA) have been studied (Pethe et al., J. Inorg. Biochem. 88:397-402, 2002). Among all these anions, only F⁻ exhibited a clear inhibitory effect at the mM level. Inhibition of RLA by F⁻ is reversible and uncompetitive towards L-Arg binding with a K(i) value of 1.3+/−0.5 mM at pH 7.4. This effect is dependent on pH as the IC₅₀ value of F-towards RLA increases from 1.2 to 19 mM when increasing the pH from 7 to 10. Another study has confirmed that fluoride is an uncompetitive inhibitor of rat liver arginase. This study also showed that fluoride caused substrate inhibition of rat liver arginase at substrate concentrations above 4 mM (Tormanen C D, J. Inorg. Biochem. 93:243-246, 2003).

N(omega)-Hydroxy-L-arginine (L-NOHA) is one of the most powerful arginase inhibitors reported so far (Ki=150 μM). The other products of NO synthase are either without effect (NO₂⁻, NO₃⁻) or much weaker inhibitors (L-Cit and NO) of arginase. Products derived from a possible hydrolysis of L-Arg (L-Orn and urea) or of L-NOHA (L-Cit, hydroxyurea and hydroxylamine) are also inactive toward arginase at concentrations up to 2 mM. The configuration of L-NOHA is important as D-NOHA is much less active, and its free —COOH and alpha-NH2 functions are required for recognition of liver arginase. L-NOHA is also a potent inhibitor of the arginase activity of rat liver homogenates and of murine macrophages (IC₅₀ of 150 and 450 μM, respectively) (Buga et al., Am. J. Physiol., 271: H1988-1998, 1996).

Another specific inhibitor of arginase, N(omega)-hydroxy-L-nor-arginine (nor-NOHA), is about 40-fold more potent than L-NOHA in inhibiting the hydrolysis of L-arginine to L-ornithine catalyzed by unstimulated murine macrophages (IC₅₀ values 12+/−5 and 400+/−50 μM, respectively). Stimulation of murine macrophages with interferon-ganuna and lipopolysaccharide (IFN-gamma+LPS) results in clear expression of an inducible NOS (iNOS) and to an increase in arginase activity. Nor-NOHA is also a potent inhibitor of arginase in IFN-gamma+LPS-stimulated macrophage (IC50 value 10+/−3 μM). In contrast to NOHA, nor-NOHA is neither a substrate nor an inhibitor for iNOS and it appears as a useful tool to study the interplays between arginase and NOS (Tenu et al., Nitric Oxide, 3: 427-438, 1999).

Other arginase inhibitors found in recent years include: N omega-hydroxy-D,L-indospicine and 4-hydroxyaridino-D,L-phenylalanine, which inhibit both liver arginase and arginase in the alveolar macrophages (Hey et al., Br. J. Pharmacol. 121:395-400, 1997); 2(S)-amino-6-boronohexanoic acid (ABH), which was found to be approximately 250 times more potent than L-NOHA in inhibiting the arginase activity in internal anal sphincter (Baggio et al., J. Pharmacol. Exp. Ther. 290:1409-1416, 1999); and α-difluoromethylornithine (DFMO), which is commonly used as a specific ornithine decarboxylase irreversible inhibitor but also shows an inhibitive effect on the flux of L-arginine through arginase in intact cells (Selamnia et al., Biochem. Pharmacol. 55:1241-1245, 1998). These arginase inhibitors may have important pathophysiological and therapeutic implications in diseases involving elevated arginase activity.

An alternative to direct inhibition of arginase activity is the inhibition of signal transduction pathways leading to the activation of arginase activity or arginase expression. For example, pathogenesis relating to elevated arginase activity may be ameliorated by the administration of IL-13 receptor (IL-13R). As described earlier, IL-13 is an immunoregulatory cytokine secreted predominantly by activated TH2 cells. Over the past several years, it has become evident that IL-13 is a key mediator in the pathogenesis of allergic inflammation. In mice, IL-13 mediated signaling is sufficient to elicit all four asthma-related pathophysiological phenotypes and is required for the hypersecretion of mucus and induced AHR. Given the importance of IL-13 as an effector molecule, regulation at the level of its receptors might be an important mechanism of modulating IL-13 responses and hence the propagation of the allergic response. IL-13 shares a common receptor subunit with IL-4, i.e., the alpha subunit of the IL-4 receptor (IL-4Rα). Characterization of IL-13-deficient mice, IL-4-deficient mice, and IL-4 receptor alpha-deficient (IL-4Rα(−/−)) mice have demonstrated nonredundant roles for IL-13. IL-13 mediates its effects by interacting with a complex receptor system comprised of IL-4Rα and two IL-13 binding proteins, IL-13Rα1 and IL-13Rα2. IL-13 receptors are expressed on human B cells, basophils, eosinophils, mast cells, endothelial cells, fibroblasts, monocytes, macrophages, respiratory epithelial cells, and smooth muscle cells.

However, functional IL-13 receptors have not been demonstrated on human or mouse T cells. Unlike IL-4, IL-13 does not appear to be important in the initial differentiation of CD4 T cells into TH2-type cells but rather appears to be important in the effector phase of allergic inflammation. This assessment is further supported by many in vivo observations, including that administration of IL-13 resulted in allergic inflammation, tissue-specific overexpression of IL-13 in the lungs of transgenic mice resulted in airway inflammation and mucus hypersecretion, IL-13 blockade abolished allergic inflammation independently of IL-4, and IL-13 appears to be more important than IL-4 in mucus hypersecretion. Accordingly, IL-13 is an attractive, novel therapeutic target for pharmacologic intervention in allergic disorders. Administration of IL-13R could potentially inhibit or even block the IL-13 signaling pathway, prevent IL-13-induced ARG1 expression, and ameliorate asthma-related pathologies.

### ARG1 and CAT2 as Markers for Inflammatory Diseases

The present invention identifies that CAT2 and ARG1 are over-expressed in the lung tissue of an animal model of asthma. Accordingly, these genes or their expression products can be used as markers for inflammatory diseases such as asthma or COPD. The expression levels of these genes can be detected by using, for example, RT-PCR, nucleic acid arrays, or immunoassays. Examples of immunoassay formats include, but are not limited to, latex or other particle agglutination, electrochemiluminescence, ELISAs, RIAs, sandwich or immunometric assays, time-resolved fluorescence, lateral flow assays, fluorescence polarization, flow cytometry, immunohistochemical assays, Western blots, and proteomic chips. CAT2 and ARG1 protein or mRNA levels can be detected in body fluids or tissue samples.

The markers can be used to provide diagnosis or prognosis information in a particular subject sample or to assess the efficacy of a treatment or therapy of inflammatory diseases. For example, comparison of expression levels of CAT2 and ARG1 at different stages of the disease progression provides a method for long-term prognosis, including survival. CAT2 and ARG1 gene polymorphism may also be indicative of a subject's susceptibility to inflammatory diseases.

In another example, the efficacy of a particular treatment regime can be evaluated, including whether a particular drug will act to improve the long-term prognosis in a particular patient. Asthma, COPD, and arthritis are complex diseases whose clinical manifestations are diverse and variable. Patients vary both with respect to disease course and response to available therapy, and these variations most probably reflect differences in type of the disease. Therefore, an added utility of the current invention is to provide methods of identifying patients most likely to respond to a treatment course.

Although the initial differentiation expression analysis was performed in a mouse model, it is well-appreciated that a dysfunctional gene that leads to disease in animals can also, when dysfunctional in human, lead to a similar syndrome in humans. It is thus specifically intended by the present invention and understood that the present invention specifically encompasses human CAT2 and ARG1 genes. CAT2 and ARG1 homologs from other organisms may also be useful in the use of animal models for the study of asthma, COPD, or other inflammatory diseases. ARG1 and CAT2 homologs from other organisms may be obtained by using any method known in the art.

### Treating Inflammatory Diseases by Inhibiting CAT2 or ARG1 Activities

CAT2 or ARG1 genomic sequences, promoters, exons, introns, RNA transcripts, or encoded proteins can be targets for a treatment or therapeutic agent. They can also be used to generate gene therapy vectors that inhibit CAT2 and/or ARG1 expression or CAT2 and/or ARG1 protein activities.

Without limitation as to mechanism, the invention is based in part on the principle that inhibition of CAT2 and/or ARG1 expression or activity may ameliorate inflammatory diseases such as asthma or COPD. CAT2 and/or ARG1 inhibitors may also be efficacious in treating fibrosis, airway hyperresponsiveness, goblet cell hyperplasia, airway inflammation, and oxidative stress. The inhibition may occur at transcriptional, post-transcriptional, translational, or post-translational levels. For example, a CAT2 or ARG1 promoter or mRNA can be targeted to inhibit transcription or translation, respectively. The post-translational processing of CAT2 or ARG1 proteins, such as glycosylation and dimerization, may also be targeted.

The discovery of the CAT2 and ARG1 expression pattern in the mouse model of asthma allows for the screening of test agents with the goal of modulating CAT2 and/or ARG1 expression or CAT2 and/or ARG1 activities. The test agents may be screened by their effect on CAT2 and/or ARG1 expression at the mRNA or protein level, or by their effect on the activity of the CAT2 and/or ARG1 gene products.

In another embodiment, a modulator of CAT2 and/or ARG1 expression or CAT2 and/or ARG1 activities may be used as a therapeutic agent for asthma, COPD and other inflammatory diseases. The modulator may be a polynucleotide such as a ribozyme or an RNAi, a polypeptide such as CAT2 and/or ARG1 mutant having a dominant negative effect on an activity of the wild-type CAT2 and/or ARG1, a viral or non-viral gene therapy vector, or any other small molecule or biomolecule that is capable of inhibiting CAT2 and/or ARG1 activity or CAT2 and/or ARG1 gene expression. Such a modulator can be formulated into pharmaceutical compositions for use in the present invention.

### Probes, Primers, Antisense and RNAi Sequences

One aspect of the invention pertains to polynucleotide probes or primers for detecting or quantitating CAT2 or ARG1 gene products in biological samples. CAT2 or ARG1 probes/primers can be derived from any portion of CAT2 or ARG1 genes. The probes/primers can have any desirable length. For instance, the probes can have 15, 20, 25, 50, 75, 100, 125, 150, 175, 200, 225, 250, 275, 300, 325, 350, 400 or more consecutive nucleotides. In many embodiments, the probes can hybridize under stringent or highly stringent conditions to a RNA transcript, or the complement thereof, of CAT2 or ARG1 genes.

Examples of conditions of different hybridization stringency are listed in Table 1. Highly stringent conditions are those that are at least as stringent as conditions A-F; stringent conditions are at least as stringent as conditions G-L; and reduced stringency conditions are at least as stringent as conditions M-R. As used in Table 1, hybridization is carried out under a given hybridization condition for about 2 hours, followed by two 15-minute washes under the corresponding washing condition(s).

TABLE 1
Stringency Conditions

| | Poly- | | | |
|---|---|---|---|---|
| Stringency | nucleotide | Hybrid | Hybridization Temperature and | Wash Temp. |
| Condition | Hybrid | Length (bp)¹ | Buffer^{H} | and Buffer^{H} |
| A | DNA:DNA | >50 | 65° C.; 1xSSC -or- | 65° C.; 0.3xSSC |
| | | | 42° C.; 1xSSC, 50% formamide | |
| B | DNA:DNA | <50 | T_{B}*; 1xSSC | T_{B}*; 1xSSC |
| C | DNA:RNA | >50 | 67° C.; 1xSSC -or- | 67° C.; 0.3xSSC |
| | | | 45° C.; 1xSSC, 50% formamide | |
| D | DNA:RNA | <50 | T_{D}*; 1xSSC | T_{D}*; 1xSSC |
| E | RNA:RNA | >50 | 70° C.; 1xSSC -or- | 70° C.; 0.3xSSC |
| | | | 50° C.; 1xSSC, 50% formamide | |
| F | RNA:RNA | <50 | T_{F}*; 1xSSC | T_{F}*; 1xSSC |
| G | DNA:DNA | >50 | 65° C.; 4xSSC -or- | 65° C.; 1xSSC |
| | | | 42° C.; 4xSSC, 50% formamide | |
| H | DNA:DNA | <50 | T_{H}*; 4xSSC | T_{H}*; 4xSSC |
| I | DNA:RNA | >50 | 67° C.; 4xSSC -or- | 67° C.; 1xSSC |
| | | | 45° C.; 4xSSC, 50% formamide | |
| J | DNA:RNA | <50 | T_{J}*; 4xSSC | T_{J}*; 4xSSC |
| K | RNA:RNA | >50 | 70° C.; 4xSSC -or- | 67° C.; 1xSSC |
| | | | 50° C.; 4xSSC, 50% formamide | |
| L | RNA:RNA | <50 | T_{L}*; 2xSSC | T_{L}*; 2xSSC |
| M | DNA:DNA | >50 | 50° C.; 4xSSC -or- | 50° C.; 2xSSC |
| | | | 40° C.; 6xSSC, 50% formamide | |
| N | DNA:DNA | <50 | T_{N}*; 6xSSC | T_{N}*; 6xSSC |
| O | DNA:RNA | >50 | 55° C.; 4xSSC -or- | 55° C.; 2xSSC |
| | | | 42° C.; 6xSSC, 50% formamide | |
| P | DNA:RNA | <50 | T_{P}*; 6xSSC | T_{P}*; 6xSSC |
| Q | RNA:RNA | >50 | 60° C.; 4xSSC -or- | 60° C.; 2xSSC |
| | | | 45° C.; 6xSSC, 50% formamide | |
| R | RNA:RNA | <50 | T_{R}*; 4xSSC | T_{R}*; 4xSSC |

¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity.
^{H}SSPE (1xSSPE is 0.15M NaCl, 10 mM NaH₂PO₄, and 1.25 mM EDTA, pH 7.4) can be substituted for SSC (1xSSC is 0.15M NaCl and 15 mM sodium citrate) in the hybridization and wash buffers.
T_{B}*-T_{R}*: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). For hybrids between 18 and 49 base pairs in length, Tₘ(° C.) = 81.5 + 16.6(log₁₀ Na⁺) + 0.41(% G + C) − (600/N), where N is the number of bases in the hybrid, and Na⁺ is the concentration of sodium ions in the hybridization buffer (Na⁺ for 1xSSC = 0.165M).

Another aspect of the invention pertains to polynucleotides encoding CAT2 and ARG1 mutants that contain changes in amino acid residues. Such mutants may compete with the wild-type CAT2 and ARG1 proteins and inhibit the activity of the wild-type CAT2 and ARG1 proteins. An isolated polynucleotide molecule encoding a mutant CAT2 and an ARG1 gene can be created by introducing one or more nucleotide substitutions, additions or deletions into the nucleotide sequence of the polynucleotide, such that one or more amino acid substitutions, additions or deletions are introduced into the encoded protein. Such techniques are well-known in the art. Mutations can be introduced into a CAT2 and an ARG1 gene by standard techniques, such as site-directed mutagenesis and PCR-mediated mutagenesis. Alternatively, mutations can be introduced randomly along all or part of a coding sequence of the CAT2 and ARG1 gene or cDNA, such as by saturation mutagenesis, and the resultant mutants can be screened for biological activity to identify mutants that are capable of inhibiting wild-type protein activity (the dominant negative mutant). Following mutagenesis, the encoded protein can be expressed recombinantly and the activity of the protein can be determined.

A polynucleotide may be further modified to increase stability in vivo. Possible modifications include, but are not limited to, the addition of flanking sequences at the 5′ and/or 3′ ends; the use of phosphorothioate or 2-o-methyl rather than phosphodiesterase linkages in the backbone; and/or the inclusion of nontraditional bases such as inosine, queosine and wybutosine, as well as acetyl-methyl-, thio- and other modified forms of adenine, cytidine, guanine, thymine and uridine.

Another aspect of the invention pertains to isolated polynucleotide molecules that are antisense to CAT2 or ARG1 genes or their transcripts. An “antisense” polynucleotide comprises a nucleotide sequence which is complementary to a “sense” polynucleotide encoding a protein, e.g., complementary to the coding strand of a double-stranded cDNA molecule or complementary to an mRNA sequence. Accordingly, an antisense polynucleotide can form hydrogen bonds with a sense polynucleotide. The antisense polynucleotide can be complementary to an entire coding strand of the CAT2 or ARG1 gene of the invention or to only a portion thereof. In one embodiment, an antisense polynucleotide molecule is antisense to a “coding region” of the coding strand of a nucleotide sequence of the invention. In another embodiment, the antisense polynucleotide molecule is antisense to a “noncoding region” of the coding strand of a nucleotide sequence of the invention.

Antisense polynucleotides of the invention can be designed according to the rules of Watson and Crick base pairing. The antisense polynucleotide molecule can be complementary to the entire coding region of an mRNA corresponding to a gene of the invention. It can also be an oligonucleotide which is antisense to only a portion of the coding or noncoding region. An antisense oligonucleotide can be, for example, about 5, 10, 15, 20, 25, 30, 35, 40, 45 or 50 nucleotides in length. An antisense polynucleotide of the invention can be constructed using chemical synthesis and enzymatic ligation reactions using procedures known in the art. For example, an antisense polynucleotide (e.g., an antisense oligonucleotide) can be chemically synthesized using naturally occurring nucleotides or variously modified nucleotides designed to increase the biological stability of the molecules or to increase the physical stability of the duplex formed between the antisense and sense polynucleotides, e.g., phosphorothioate derivatives and acridine substituted nucleotides can be used. Examples of modified nucleotides which can be used to generate the antisense polynucleotide include 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenosine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, (acp3)w, and 2,6-diaminopurine. Alternatively, the antisense polynucleotide can be produced biologically using an expression vector into which a polynucleotide has been subcloned in an antisense orientation (i.e., RNA transcribed from the inserted polynucleotide will be of an antisense orientation to a target polynucleotide of interest, described further in the following subsection).

The antisense polynucleotide molecules of the invention are typically administered to a subject or generated in situ such that they hybridize with or bind to cellular mRNA and/or genomic DNA encoding a CAT2 and an ARG1 protein to thereby inhibit expression of the protein, e.g., by inhibiting transcription and/or translation. The hybridization can be by conventional nucleotide complementarity to form a stable duplex, or, for example, in the cases of an antisense polynucleotide molecule which binds to DNA duplexes, through specific interactions in the major groove of the double helix. An example of a route of administration of antisense polynucleotide molecules of the invention include direct injection at a tissue site (e.g., intestine or blood). Alternatively, antisense polynucleotide molecules can be modified to target selected cells and then administered systemically. For example, for systemic administration, antisense molecules can be modified such that they specifically bind to receptors or antigens expressed on a selected cell surface, e.g., by linking the antisense polynucleotide molecules to peptides or antibodies which bind to cell surface receptors or antigens. The antisense polynucleotide molecules can also be delivered to cells using the vectors described herein. To achieve sufficient intra-cellular concentrations of the antisense molecules, vector constructs in which the antisense polynucleotide molecule is placed under the control of a strong pol II or pol III promoter can be used.

Another aspect of the invention pertains to an α-anomeric polynucleotide molecule. The α-anomeric polynucleotide molecule is capable of forming specific double-stranded hybrids with a CAT2 and an ARG1 RNA in which, contrary to the usual β-units, the strands run parallel to each other. The α-anomeric polynucleotide molecule can also comprise a 2-o-methylribonucleotide or a chimeric RNA-DNA analogue.

In another embodiment, the isolated polynucleotide is a ribozyme. Ribozymes are catalytic RNA molecules with ribonuclease activity which are capable of cleaving a single-stranded polynucleotide, such as an mRNA, to which they have a complementary region. Thus, ribozymes can be used to catalytically cleave mRNA transcripts of the CAT2 and/or ARG1 gene to thereby inhibit translation of said mRNA. A ribozyme having specificity for the CAT2 and ARG1 gene can be designed based upon the nucleotide sequence of the CAT2 and ARG1 gene. An mRNA transcribed from the CAT2 and ARG1 gene can be used to select a catalytic RNA having a specific ribonuclease activity from a pool of RNA molecules. Alternatively, expression of the CAT2 and ARG1 gene can be inhibited by targeting nucleotide sequences complementary to the regulatory region of these genes (e.g., the promoter and/or enhancers) to form triple helical structures that prevent transcription of the gene in target cells.

Expression of the CAT2 and ARG1 gene can also be inhibited using RNA interference (RNAi). This is a technique for post-transcriptional gene silencing (“PTGS”), in which target gene activity is specifically abolished with cognate double-stranded RNA (“dsRNA”). In many embodiments, dsRNA of about 21 nucleotides, homologous to the target gene, is introduced into the cell and a sequence specific reduction in gene activity is observed. RNA interference provides a mechanism of gene silencing at the mRNA level. It offers an efficient and broadly applicable approach for gene knock-out as well as for therapeutic purposes.

Sequences capable of inhibiting gene expression by RNA interference can have any desired length. For instance, the sequence can have at least 15, 20, 25, or more consecutive nucleotides. The sequence can be dsRNA or any other type of polynucleotide, provided that the sequence can form a functional silencing complex to degrade the target mRNA transcript.

In one embodiment, the sequence comprises or consists of a short interfering RNA (siRNA). The siRNA can be, for example, dsRNA having 19-25 nucleotides. siRNAs can be produced endogenously by degradation of longer dsRNA molecules by an RNase III-related nuclease called Dicer. siRNAs can also be introduced into a cell exogenously or by transcription of an expression construct. Once formed, the siRNAs assemble with protein components into endoribonuclease-containing complexes known as RNA-induced silencing complexes (RISCs). An ATP-generated unwinding of the siRNA activates the RISCs, which in turn target the complementary mRNA transcript by Watson-Crick base-pairing, thereby cleaving and destroying the mRNA. Cleavage of the mRNA takes place near the middle of the region bound by the siRNA strand. This sequence-specific mRNA degradation results in gene silencing.

At least two ways can be employed to achieve siRNA-mediated gene silencing. First, siRNAs can be synthesized in vitro and introduced into cells to transiently suppress gene expression. Synthetic siRNA provides an easy and efficient way to achieve RNAi. siRNA are duplexes of short mixed oligonucleotides which can include, for example, 19 nucleotides with symmetric dinucleotide 3′ overhangs. Using synthetic 21 bp siRNA duplexes (e.g., 19 RNA bases followed by a UU or dTdT 3′ overhang), sequence-specific gene silencing can be achieved in mammalian cells. These siRNAs can specifically suppress targeted gene translation in mammalian cells without activation of DNA-dependent protein kinase (PKR) by longer dsRNA, which may result in non-specific repression of translation of many proteins.

Second, siRNAs can be expressed in vivo from vectors. This approach can be used to stably express siRNAs in cells or transgenic animals. In one embodiment, siRNA expression vectors are engineered to drive siRNA transcription from polymerase III (po III) transcription units. Pol III transcription units are suitable for hairpin siRNA expression, since they deploy a short AT rich transcription termination site that leads to the addition of 2 bp overhangs (e.g., UU) to hairpin siRNAs—a feature that is helpful for siRNA function. The Pol III expression vectors can also be used to create transgenic mice that express siRNA.

In another embodiment, siRNAs can be expressed in a tissue-specific manner. Under this approach, long double-stranded RNAs (dsRNAs) are first expressed from a tissue-specific promoter in the nuclei of selected cell lines or transgenic mice. The long dsRNAs are processed into siRNAs in the nuclei (e.g., by Dicer). The siRNAs exit from the nuclei and mediate gene-specific silencing. A similar approach can be used in conjunction with tissue-specific promoters to create tissue-specific knockdown mice. Any 3′ dinucleotide overhang, such as UU, can be used for siRNA design. In some cases, G residues in the overhang are avoided because of the potential for the siRNA to be cleaved by RNase at single-stranded G residues. With regard to the siRNA sequence itself, it has been found that siRNAs with 30-50% GC content can be more active than those with a higher G/C content in certain cases. Moreover, since a 4-6 nucleotide poly(T) tract may act as a termination signal for RNA pol III, stretches of >4 Ts or As in the target sequence may be avoided in certain cases when designing sequences to be expressed from an RNA pol III promoter. In addition, some regions of mRNA may be either highly structured or bound by regulatory proteins. Thus, it may be helpful to select siRNA target sites at different positions along the length of the gene sequence. Finally, the potential target sites can be compared to the appropriate genome database (human, mouse, rat, etc.). Any target sequences with more than 16-17 contiguous base pairs of homology to other coding sequences may be eliminated from consideration in certain cases.

In one embodiment, siRNA is designed to have two inverted repeats separated by a short spacer sequence and end with a string of Ts that serve as a transcription termination site. This design produces an RNA transcript that is predicted to fold into a short hairpin siRNA. The selection of siRNA target sequence, the length of the inverted repeats that encode the stem of a putative hairpin, the order of the inverted repeats, the length and composition of the spacer sequence that encodes the loop of the hairpin, and the presence or absence of 5′-overhangs, can vary to achieve desirable results.

The siRNA targets can be selected by scanning an mRNA sequence for AA dinucleotides and recording the 19 nucleotides immediately downstream of the AA. Other methods can also been used to select the siRNA targets. In one example, the selection of the siRNA target sequence is purely empirically determined (see, e.g., Sui et al, Proc. Natl. Acad. Sci. USA 99: 5515-5520, 2002), as long as the target sequence starts with GG and does not share significant sequence homology with other genes as analyzed by BLAST search. In another example, a more elaborate method is employed to select the siRNA target sequences. This procedure exploits an observation that any accessible site in endogenous mRNA can be targeted for degradation by synthetic oligodeoxyribonucleotide/Rnase H method (Lee et al, Nature Biotechnol 20: 500-505, 2002).

In another embodiment, the hairpin siRNA expression cassette is constructed to contain the sense strand of the target, followed by a short spacer, the antisense strand of the target, and 5-6 Ts as transcription terminator. The order of the sense and antisense strands within the siRNA expression constructs can be altered without affecting the gene silencing activities of the hairpin siRNA. In certain instances, the reversal of the order may cause partial reduction in gene silencing activities.

The length of nucleotide sequence being used as the stem of siRNA expression cassette can range, for instance, from 19 to 29. The loop size can range from 3 to 23 nucleotides. Other lengths and/or loop sizes can also be used.

In yet another embodiment, a 5′ overhang in the hairpin siRNA construct can be used, provided that the hairpin siRNA is functional in gene silencing. In one example, the 5′ overhang includes about 6 nucleotide residues.

In still yet another embodiment, the target sequences for RNAi are about 21-mer sequence fragments selected from the CAT2 and ARG1 coding sequences, such as SEQ ID NOS:1 and 5. The target sequences can be selected from either ORF regions or non-ORF regions. The 5′ end of each target sequence has dinucleotide “NA,” where “N” can be any base and “A” represents adenine. The remaining 19-mer sequence has a GC content of between 30% and 65%. In many examples, the remaining 19-mer sequence does not include any four consecutive A or T (i.e., AAAA or TTTT), three consecutive G or C (i.e., GGG or CCC), or seven “GC” in a row. Examples of the target sequences prepared using the above-described criteria (“Relaxed Criteria”) are illustrated in Table 2. Each target sequence in Table 2 has SEQ ID NO:3n, and the corresponding siRNA sense and antisense strands have SEQ ID NO:3n+1 and SEQ ID NO:3n+2, respectively, where n is a positive integer. For each CAT2 and ARG1 coding sequence (e.g. SEQ ID NOS: 1 and 5, respectively), multiple target sequences can be selected.

Additional criteria can be used for RNAi target sequence design. In one example, the GC content of the remaining 19-mer sequence is limited to between 35% and 55%, and any 19-mer sequence having three consecutive A or T (i.e., AAA or TTT) or a palindrome sequence with 5 or more bases is excluded. In addition, the 19-mer sequence can be selected to have low sequence homology to other human genes. In one embodiment, potential target sequences are searched by BLASTN against NCBI's human UniGene cluster sequence database. The human UniGene database contains non-redundant sets of gene-oriented clusters. Each UniGene cluster includes sequences that represent a unique gene. 19-mer sequences producing no hit to other human genes under the BLASTN search can be selected. During the search, the e-value may be set at a stringent value (such as “1”). Furthermore, the target sequence can be selected from the ORF region, and is at least 75-bp from the start and stop codons. Examples of the target sequences prepared using these criteria (“Stringent Criteria”) are demonstrated in Table 2 (SEQ ID NO:3n, where n is a positive integer). siRNA sense and antisense sequences (SEQ ID NO:3n+1 and SEQ ID NO:3n+2, respectively) for each target sequence (SEQ ID NO:3n) are also provided.

TABLE 2
RNAi Target Sequences and siRNA Sequences

| | Relaxed Criteria | Stringent Criteria |
|---|---|---|
| | (target: SEQ ID NO: 3n; | (target: SEQ ID NO: 3n; |
| SEQ ID NO | siRNA sense: SEQ ID NO: 3n + 1; | siRNA sense: SEQ ID NO: 3n + 1; |
| (coding sequences) | siRNA antisense: SEQ ID NO: 3n + 2) | siRNA antisense: SEQ ID NO: 3n + 2) |
| SEQ ID NO: 1 | SEQ ID NOS: 9-725 | SEQ ID NOS: 1,332-1,409 |
| SEQ ID NO: 4 | SEQ ID NOS: 726-1,331 | SEQ ID NOS: 1,410-1,517 |

The effectiveness of the siRNA sequences can be evaluated using various methods known in the art. For instance, an siRNA sequence of the present invention can be introduced into a cell that over-expresses a CAT2 or ARG1 gene. The polypeptide or mRNA level of the CAT2 or ARG1 in the cell can be detected. A substantial change in the expression level of the LRG before and after the introduction of the siRNA sequence is indicative of the effectiveness of the siRNA sequence in suppressing the expression of the CAT2 or ARG1 gene. In one example, the expression levels of other genes are also monitored before and after the introduction of the siRNA sequence. An siRNA sequence which has inhibitory effect on the CAT2 or ARG1 expression but does not significantly affect the expression of other genes can be selected. In another example, multiple siRNA or other RNAi sequences can be introduced into the same target cell. These siRNA or RNAi sequences specifically inhibit the CAT2 or ARG1 gene expression but not the expression of other genes. In yet another example, siRNA or other RNAi sequences that inhibit the expression of both the CAT2 or ARG1 gene and other gene or genes can be used.

In yet another embodiment, the polynucleotide molecules of the present invention can be modified at the base moiety, sugar moiety or phosphate backbone to improve, e.g., the stability, hybridization, or solubility of the molecule. For example, the deoxyribose phosphate backbone of the polynucleotide molecules can be modified to generate peptide polynucleotides. As used herein, the terms “peptide polynucleotides” or “PNAs” refer to polynucleotide mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols.

PNAs can be used in therapeutic and diagnostic applications. For example, PNAs can be used as antisense agents for sequence-specific inhibition of CAT2 or ARG1 expression by, for example, inducing transcription or translation arrest or inhibiting replication. PNAs of the polynucleotide molecules of the invention can also be used in the analysis of single base pair mutations in a gene e.g., by PNA-directed PCR clamping, as artificial restriction enzymes when used in combination with other enzymes (e.g., S1 nucleases) or as probes or primers for DNA sequencing or hybridization.

In another embodiment, PNAs can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of the polynucleotide molecules of the invention can be generated which may combine the advantageous properties of PNA and DNA. Such chimeras allow DNA recognition enzymes, (e.g., DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation. The synthesis of PNA-DNA chimeras can be performed. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a spacer between the PNA and the 5′ end of DNA. PNA monomers are then coupled in a stepwise manner to produce a chimeric molecule with a 5′ PNA segment and a 3′ DNA segment. Alternatively, chimeric molecules can be synthesized with a 5′ DNA segment and a 3′ PNA segment.

### CAT2 and ARG1 Polypeptides

Several aspects of the invention pertain to mutated CAT2 and ARG1 polypeptides capable of inhibiting normal CAT2 or ARG1 polypeptide activity, as well as polypeptide fragments suitable for use as immunogens to raise anti-CAT2 or anti-ARG1 antibodies. In one embodiment, mutated CAT2 and ARG1 polypeptides (e.g., dominant-negative mutants) are produced by recombinant DNA techniques. Alternatively, mutated CAT2 and ARG1 polypeptides can be synthesized chemically using standard peptide synthesis techniques.

The present invention also pertains to variants of a CAT2 or an ARG1 polypeptide which function as antagonists to the CAT2 or ARG1 polypeptide. In one embodiment, antagonists or agonists of CAT2 or ARG1 polypeptides are used as therapeutic agents. For example, antagonists to a CAT2 or an ARG1 polypeptide can decrease the activity of the CAT2 or ARG1 protein and ameliorate an inflammatory disease in a subject wherein the CAT2 or ARG1 protein is over-expressed. Variants of CAT2 or ARG1 polypeptide can be generated by mutagenesis, e.g., discrete point mutation or truncation of a CAT2 or an ARG1 gene.

In certain embodiments, an antagonist of a CAT2 or ARG1 polypeptide can inhibit one or more of the activities of the naturally occurring form of the CAT2 or ARG1 polypeptide by, for example, competitively modulating an activity of the CAT2 or ARG1 polypeptide. Thus, specific biological effects can be elicited by treatment with a variant of limited function.

Mutants of a CAT2 or an ARG1 polypeptide which function as either CAT2 or ARG1 polypeptide agonists or as CAT2 or ARG1 polypeptide antagonists can be identified by screening combinatorial libraries of mutants. In certain embodiments, such variants may be used, for example, as a therapeutic protein of the invention. A variegated library of CAT2 or ARG1 polypeptide variants can be produced by, for example, enzymatically ligating a mixture of synthetic oligonucleotides into gene sequences such that a degenerate set of potential CAT2 or ARG1 polypeptide sequences is expressible as individual polypeptides, or alternatively, as a set of larger fusion proteins (e.g., for phage display) containing the set of CAT2 or ARG1 polypeptide sequences therein. There are a variety of methods which can be used to produce libraries of potential CAT2 or ARG1 polypeptide variants from a degenerate oligonucleotide sequence. Chemical synthesis of a degenerate gene sequence can be performed in an automatic DNA synthesizer, and the synthetic gene is then ligated into an appropriate expression vector. Use of a degenerate set of genes allows for the provision, in one mixture, of all of the sequences encoding the desired set of potential CAT2 or ARG1 polypeptide sequences. Methods for synthesizing degenerate oligonucleotides are known in the art.

In addition, libraries of fragments of a protein coding sequence corresponding to a CAT2 or an ARG1 gene can be used to generate a variegated population of CAT2 or ARG1 polypeptide fragments for screening and subsequent selection of variants of a CAT2 or an ARG1 polypeptide. In one embodiment, a library of coding sequence fragments can be generated by treating a double-stranded PCR fragment of a CAT2 or an ARG1 gene-coding sequence with a nuclease under conditions wherein nicking occurs only about once per molecule, denaturing the double-stranded DNA, renaturing the DNA to form double-stranded DNA which can include sense/antisense pairs from different nicked products, removing single-stranded portions from reformed duplexes by treatment with S1 nuclease, and ligating the resulting fragment library into an expression vector. By this method, an expression library can be derived which encodes N-terminal, C-terminal and internal fragments of various sizes of the CAT2 or ARG1 polypeptide.

Several techniques are known in the art for screening gene products of combinatorial libraries made by point mutations or truncation, and for screening cDNA libraries for gene products having a selected property. The most widely used techniques, which are amenable to high-throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify CAT2 or ARG1 polypeptide variants (Delgrave et al., Protein Engineering 6:327-331, 1993).

Portions of a CAT2 or an ARG1 polypeptide or variants of a CAT2 or an ARG1 polypeptide having less than about 100 amino acids, and generally less than about 50 amino acids, may also be generated by synthetic means, using techniques well-known to those of ordinary skill in the art. For example, such polypeptides may be synthesized using any of the commercially available solid-phase techniques, such as the Merrifield solid-phase synthesis method, where amino acids are sequentially added to a growing amino acid chain. Equipment for automated synthesis of polypeptides is commercially available from suppliers such as Perkin Elmer/Applied BioSystems Division (Foster City, Calif.), and may be operated according to the manufacturer's instructions.

Methods and compositions for screening for protein inhibitors or activators are known in the art (see U.S. Pat. Nos. 4,980,281, 5,266,464, 5,688,635, and 5,877,007, which are incorporated herein by reference).

### Antibodies

In accordance with another aspect, antibodies specific to CAT2 or ARG1 proteins can be prepared. In many embodiments, the antibodies of the present invention can bind to CAT2 or ARG1 proteins with binding affinities of no less than than 10⁵ M⁻¹. The antibodies can be, without limitation, monoclonal, polyclonal, chimeric, humanized, scFv, Fv, Fab′, Fab, or F(ab′)₂.

A full-length CAT2 or ARG1 protein can be used or, alternatively, the invention provides antigenic peptide fragments of the CAT2 or ARG1 protein for use as immunogens. In many embodiments, the antigenic peptides of the CAT2 or ARG1 protein comprise at least 8 amino acid residues, and encompass epitopes of the CAT2 or ARG1 protein such that an antibody raised against the peptide forms a specific immune complex with the CAT2 or ARG1 protein. In many other embodiments, the antigenic peptide comprises at least 8, 12, 16, 20 or more amino acid residues.

Immunogenic portions (epitopes) may generally be identified using well-known techniques. Such techniques include screening polypeptides for the ability to react with antigen-specific antibodies, antisera and/or T-cell lines or clones. Such antisera and antibodies may be prepared as described herein, and using well-known techniques. An epitope of the CAT2 or ARG1 protein is a portion that reacts with such antisera and/or T-cells at a level that is not substantially less than the reactivity of the full-length polypeptide (e.g., in an ELISA and/or T-cell reactivity assay). Such epitopes may react within such assays at a level that is similar to or greater than the reactivity of the full-length polypeptide. Such screens may generally be performed using methods well known to those of ordinary skill in the art. For example, a polypeptide may be immobilized on a solid support and contacted with patient sera to allow the binding of antibodies within the sera to the immobilized polypeptide. Unbound sera may then be removed and bound antibodies detected using, for example, ¹²⁵I-labeled Protein A.

Exemplary epitopes encompassed by the antigenic peptide are regions of the CAT2 or ARG1 protein that are located on the surface of the polypeptide, e.g., hydrophilic regions, as well as regions with high antigenicity.

A CAT2 or ARG1 immunogen (e.g., the CAT2 or ARG1 protein, a fragment thereof, or a CAT2- or ARG1-fusion protein) typically is used to prepare antibodies by immunizing a suitable subject, (e.g., rabbit, goat, mouse or other mammal) with the immunogen. An appropriate immunogenic preparation can contain, for example, recombinantly expressed CAT2 or ARG1 immunogen or a chemically synthesized CAT2 or ARG1 immunogen. The preparation can further include an adjuvant, such as Freund's complete or incomplete adjuvant, or similar immunostimulatory agent. Immunization of a suitable subject with the immunogenic preparation induces an anti-CAT2 or -ARG1 antibody response. Techniques for preparing, isolating and using monoclonal and polyclonal anti-CAT2 or -ARG1 antibodies are well-known in the art.

Accordingly, another aspect of the invention pertains to monoclonal or polyclonal anti-CAT2 or -ARG1 antibodies. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind to CAT2 or ARG1 protein.

The anti-CAT2 or -ARG1 antibody titer in the immunized subject can be monitored over time by standard techniques, such as with an enzyme linked immunosorbent assay (ELISA) using immobilized CAT2 or ARG1 protein or a fragment of CAT2 or ARG1 protein. If desired, the antibody molecules directed against CAT2 or ARG1 protein can be isolated from the mammal (e.g., from the blood) and further purified by well-known techniques, such as protein A chromatography, to obtain the IgG fraction. At an appropriate time after immunization, e.g., when the anti-CAT2 or -ARG1 antibody titers are the highest, antibody-producing cells can be obtained from the subject and used to prepare monoclonal antibodies by standard techniques, such as the hybridoma technique, human B cell hybridoma technique, the EBV-hybridoma technique, or trioma techniques. The technology for producing monoclonal antibody hybridomas is well-known.

Any of the many well-known protocols used for fusing lymphocytes and immortalized cell lines can be applied for the purpose of generating an anti-CAT2 or -ARG1 monoclonal antibody. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Examples of immortal cell lines include mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp210-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody are detected by screening the hybridoma culture supernatants for antibodies that bind to an CAT2 or ARG1 polypeptide specifically, e.g., using a standard ELISA assay.

Alternative to preparing monoclonal antibody-secreting hybridomas, a monoclonal anti-CAT2 or -ARG1 antibody can be identified and isolated by screening a recombinant combinatorial immunoglobulin library (e.g., an antibody phase display library) with the CAT2 or ARG1 protein to thereby isolate immunoglobulin library members that bind to the CAT2 or ARG1 protein. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612).

The anti-CAT2 or -ARG1 antibodies also include “Single-chain Fv” or “scFv” antibody fragments. The scFv fragments comprise the V_{H} and V_{L} domains of an antibody, wherein these domains are present in a single polypeptide chain. Generally, the Fv polypeptide further comprises a polypeptide linker between the V_{H} and V_{L} domains that enables the scFv to form the desired structure for antigen binding.

Additionally, recombinant anti-CAT2 or -ARG1 antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

Humanized antibodies may be desirable for therapeutic treatment of human subjects. Humanized forms of non-human (e.g., murine) antibodies are chimeric molecules of immunoglobulins, immunoglobulin chains or fragments thereof (such as Fv, Fab, Fab′, F(ab′)₂ or other antigen-binding subsequences of antibodies), which contain minimal sequence derived from non-human immunoglobulin. Humanized antibodies include human immunoglobulins (recipient antibody) in which residues forming a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the constant regions being those of a human immunoglobulin consensus sequence. The humanized antibody may also include at least a portion of an immunoglobulin constant region (Fc), such as that of a human immunoglobulin.

Such humanized antibodies can be produced using transgenic mice which are incapable of expressing endogenous immunoglobulin heavy and light chain genes, but which can express human heavy and light chain genes. The transgenic mice are immunized in the normal fashion with a selected antigen, e.g., all or a portion of the CAT2 or ARG1 protein. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, Ig and IgE antibodies.

Humanized antibodies that recognize a selected epitope can be generated using a technique referred to as “guided selection.” In this approach, a selected non-human monoclonal antibody, e.g., a murine antibody, is used to guide the selection of a humanized antibody recognizing the same epitope.

In one embodiment, the antibodies to the CAT2 or ARG1 protein are capable of reducing or eliminating the biological function of the CAT2 or ARG1 protein. In many cases, at least a 25% decrease in activity can be obtained. In many other cases, at least 50%, 60%, 70%, 80%, 90%, 95% or more decrease in activity can be achieved.

An anti-CAT2 or -ARG1 antibody can be used to isolate the CAT2 or ARG1 protein or mutants of the CAT2 or ARG1 protein by standard techniques, such as affinity chromatography or immunoprecipitation. An anti-CAT2 or -ARG1 antibody can facilitate the purification of a natural or mutant CAT2 or ARG1 protein from cells and of a recombinantly produced CAT2 or ARG1 protein expressed in host cells. Moreover, an anti-CAT2 or -ARG1 antibody can be used to detect the CAT2 or ARG1 protein (e.g., in a cellular lysate or cell supernatant on the cell surface) in order to evaluate the abundance and pattern of expression of the CAT2 or ARG1 protein. Anti-CAT2 or -ARG1 antibodies can be used diagnostically to monitor protein levels in tissue as part of a clinical testing procedure to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ³⁵S and ³H.

Anti-CAT2 or -ARG1 antibodies of the invention are also useful for targeting a therapeutic to a cell or tissue having elevated CAT2 or ARG1 expression. For example, a therapeutic such as a small molecule CAT2 or ARG1 antagonist can be linked to the anti-CAT2 or anti-ARG1 antibody in order to target the therapeutic to the cell or tissue having elevated CAT2 or ARG1 expression.

A therapeutic agent may be coupled (e.g., covalently bonded) to a suitable monoclonal antibody either directly or indirectly (e.g., via a linker group). A direct reaction between an agent and an antibody is possible when each possesses a substituent capable of reacting with the other. For example, a nucleophilic group, such as an amino or sulfhydryl group, on one may be capable of reacting with a carbonyl-containing group, such as an anhydride or an acid halide, or with an alkyl group containing a good leaving group (e.g., a halide) on the other.

Alternatively, it may be desirable to couple a therapeutic agent and an antibody via a linker group. A linker group can function as a spacer to distance an antibody from an agent in order to avoid interference with binding capabilities. A linker group can also serve to increase the chemical reactivity of a substituent on an agent or an antibody, and thus increase the coupling efficiency. An increase in chemical reactivity may also facilitate the use of agents, or functional groups on agents, which otherwise would not be possible.

It will be evident to those skilled in the art that a variety of bifunctional or polyfunctional reagents, both homo- and hetero-functional (such as those described in the catalog of the Pierce Chemical Co., Rockford, Ill.), may be employed as the linker group. Coupling may be effected, for example, through amino groups, carboxyl groups, sulfhydryl groups or oxidized carbohydrate residues. There are numerous references describing such methodology, e.g., U.S. Pat. No. 4,671,958, to Rodwell et al.

Where a therapeutic agent is more potent when free from the antibody portion of the immunoconjugates of the present invention, it may be desirable to use a linker group that is cleavable during or upon internalization into a cell. A number of different cleavable linker groups have been described. The mechanisms for the intracellular release of an agent from these linker groups include cleavage by reduction of a disulfide bond (e.g., U.S. Pat. No. 4,489,710, to Spitler), by irradiation of a photolabile bond (e.g., U.S. Pat. No. 4,625,014, to Senter et al.), by hydrolysis of derivatized amino acid side chains (e.g., U.S. Pat. No. 4,638,045, to Kohn et al.), by serum complement-mediated hydrolysis (e.g., U.S. Pat. No. 4,671,958, to Rodwell et al.), and acid-catalyzed hydrolysis (e.g., U.S. Pat. No. 4,569,789, to Blattler et al.).

It may be desirable to couple more than one agent to an antibody. In one embodiment, multiple molecules of an agent are coupled to one antibody molecule. In another embodiment, more than one type of agent may be coupled to one antibody. Regardless of the particular embodiment, immunoconjugates with more than one agent may be prepared in a variety of ways. For example, more than one agent may be coupled directly to an antibody molecule, or linkers that provide multiple sites for attachment can be used.

### Vectors

Another aspect of the invention pertains to vectors containing polynucleotides encoding CAT2 and ARG1 polypeptides or portions thereof. Vectors can be plasmids or viral vectors.

The expression vectors of the invention can be designed for expression of CAT2 and ARG1 polypeptides in prokaryotic or eukaryotic cells. For example, CAT2 and ARG1 polypeptides can be expressed in bacterial cells such as E. coli, insect cells (using baculovirus expression vectors), yeast cells, or mammalian cells. In certain embodiments, such protein may be used, for example, as a therapeutic protein of the invention. Alternatively, the expression vector can be transcribed and translated in vitro, for example using T7 promoter regulatory sequences and T7 polymerase.

In another embodiment, mammalian expression vector including tissue-specific regulatory elements are used to express the polynucleotides of interest. Tissue-specific regulatory elements are known in the art and may include epithelial cell-specific promoters. Other non-limiting examples of suitable tissue-specific promoters include the liver-specific albumin promoter, lymphoid-specific promoters, promoters of T cell receptors and immunoglobulins, neuron-specific promoters (e.g., the neurofilament promoter), pancreas-specific promoters, and mammary gland-specific promoters (e.g., milk whey promoter). Developmentally-regulated promoters are also encompassed, for example the α-fetoprotein promoter.

The invention also provides a recombinant expression vector comprising a polynucleotide encoding either a CAT2 or an ARG1 polypeptide cloned into the expression vector in an antisense orientation. That is, the DNA molecule is operatively linked to a regulatory sequence in a manner which allows for expression (by transcription of the DNA molecule) of an RNA molecule which is antisense to mRNA corresponding to either a CAT2 or an ARG1 gene of the invention. Regulatory sequences operatively linked to a polynucleotide cloned in the antisense orientation can be chosen to direct the continuous expression of the antisense RNA molecule in a variety of cell types. For instance viral promoters or enhancers, or regulatory sequences can be chosen to direct constitutive, tissue specific or cell type specific expression of antisense RNA. The antisense expression vector can be in the form of a recombinant plasmid, phagemid or attenuated virus in which antisense polynucleotides are produced under the control of a high efficiency regulatory region. The activity of the promoter/enhancer can be determined by the cell type into which the vector is introduced.

The invention further provides gene delivery vehicles for delivery of polynucleotides to cells, tissues, or a mammal for expression. For example, a polynucleotide sequence of the invention can be administered either locally or systemically in a gene delivery vehicle. These constructs can utilize viral or non-viral vector approaches in in vivo or ex vivo modality. Expression of such coding sequence can be induced using endogenous mammalian or heterologous promoters. Expression of the coding sequence in vivo can be either constituted or regulated. The invention includes gene delivery vehicles capable of expressing the contemplated polynucleotides. The gene delivery vehicle can be, for example, a viral vector, such as a retroviral, lentiviral, adenoviral, adeno-associated viral (AAV), herpes viral, or alphavirus vector. The viral vector can also be an astrovirus, coronavirus, orthomyxovirus, papovavirus, paramyxovirus, parvovirus, picornavirus, poxvirus, or togavirus viral vector.

Delivery of the gene therapy constructs of this invention into cells is not limited to the above mentioned viral vectors. Other delivery methods and media may be employed such as, for example, nucleic acid expression vectors, polycationic condensed DNA linked or unlinked to killed adenovirus alone, ligand-linked DNA, liposome-DNA complex, eukaryotic cell delivery vehicles cells, deposition of photopolymerized hydrogel materials, handheld gene transfer particle gun, ionizing radiation, nucleic charge neutralization or fusion with cell membranes. Particle mediated gene transfer may be employed. Briefly, the sequence can be inserted into conventional vectors that contain conventional control sequences for high level expression, and then be incubated with synthetic gene transfer molecules such as polymeric DNA-binding cations like polylysine, protamine, and albumin, linked to cell targeting ligands such as asialoorosomucoid, insulin, galactose, lactose or transferrin. Naked DNA may also be employed. Uptake efficiency may be improved using biodegradable latex beads. DNA coated latex beads are efficiently transported into cells after endocytosis initiation by the beads. The method may be improved further by treatment of the beads to increase hydrophobicity and thereby facilitate disruption of the endosome and release of the DNA into the cytoplasm.

Another aspect of the invention pertains to the expression of either a CAT2 or an ARG1 gene using a regulatable expression system. These systems include, but are not limited to, the Tet-on/off system, the Ecdysone system, the Progesterone-system, and the Rapamycin-system.

Another aspect of the invention pertains to the use of host cells which are transformed, transfected, or transduced with vectors encoding or comprising either a CAT2 or an ARG1 polypeptide or portions thereof. The host cells can be prokaryotic or eukaryotic cells. These host cells can be employed to express any desired CAT2 or ARG1 polypeptide.

### Detection Methods

As discussed earlier, expression level of CAT2 or ARG1 gene may be used as a marker for inflammatory diseases. Detection and measurement of the relative amount of a CAT2 or an ARG1 product (polynucleotides or polypeptides) can be by any method known in the art. The detection or measurement can be qualitative or quantitative.

Typical methodologies for detection of a transcribed polynucleotide include extraction of RNA from a cell or tissue sample, followed by hybridization of a labeled probe to the extracted RNA and detection of the labeled probe (e.g., Northern blotting, or nucleic acid array).

Typical methodologies for peptide detection include protein extraction from a cell or tissue sample, followed by binding of an antibody specific for the target protein to the protein sample, and detection of the antibody. For example, detection of a CAT2 or an ARG1 polypeptide may be accomplished using either a anti-CAT2 or an anti-ARG1 polyclonal antibody. Antibodies are generally detected by the use of a labeled secondary antibody. The label can be a radioisotope, a fluorescent compound, an enzyme, an enzyme co-factor, or ligand. Such methods are well understood in the art.

In another embodiment, the detection of CAT2 or ARG1 protein expression is conducted by using small molecules that have high binding affinities to CAT2 or ARG1 protein products. In many examples, the small molecules are readily detectable. In many other examples, the small molecules can be directly or indirectly labeled by other detectable substances. Examples of these detectable substances include, without limitation, enzymes, prosthetic groups, fluorescent materials, luminescent materials, bioluminescent materials, radioactive materials, particulate materials, or colloidal metals.

In certain embodiments, the CAT2 or ARG1 gene itself may serve as a marker for inflammatory diseases. For example, an increase or decrease of genomic copies of the CAT2 or ARG1 gene, such as by duplication or deletion of the gene, may be correlated with an inflammatory disease.

Detection of specific CAT2 or ARG1 polynucleotide molecules may also be assessed by gel electrophoresis, column chromatography, or direct sequencing, quantitative PCR, RT-PCR, nested-PCR, or other techniques known in the art.

Detection of the presence or number of copies of all or a part of a CAT2 or an ARG1 gene may be performed using any method known in the art. In one embodiment, Southern analysis is employed to assess the presence and/or quantity of the genomic copies of CAT2 or ARG1 gene. Other useful methods for DNA detection and/or quantification include, but are not limited to, direct sequencing, gel electrophoresis, column chromatography, quantitative PCR, or other means as appreciated by those skilled in the art.

### Screening Methods

The invention also provides methods (also referred to herein as “screening assays”) for identifying modulators, i.e., candidate or test compounds or agents comprising therapeutic moieties (e.g., peptides, peptidomimetics, peptoids, polynucleotides, small molecules or other drugs) which (a) bind to the CAT2 or ARG1 protein, or (b) have an inhibitory effect on the activity of the CAT2 or ARG12 protein, or, more specifically, (c) have a modulatory effect on the interactions of the CAT2 or ARG1 protein with one or more of its natural substrates (e.g., peptide, protein, hormone, co-factor, or polynucleotide), or (d) have an inhibitory effect on the expression of the CAT2 or ARG1 gene. Such assays typically comprise a reaction between a CAT2 or an ARG1 gene and one or more assay components. The other components may be either the test compound itself, or a combination of test compound and a binding partner of the CAT2 or ARG1 protein.

The test compounds of the present invention are generally inorganic molecules, small organic molecules, and biomolecules. Biomolecules include, but are not limited to, amino acid, nucleic acid, lipid, sugar, steroid, polypeptides, polynucleotides, polysaccharides, as well as any naturally-occurring or synthetic organic compounds that have a bioactivity in mammals. In one embodiment the test compound is a small organic molecule. In another embodiment, the test compound is a biomolecule.

The test compounds of the present invention may be obtained from any available source, including systematic libraries of natural and/or synthetic compounds. Test compounds may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., J. Med. Chem. 37: 2678-85, 1994); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, Anticancer Drug Des. 12: 145, 1997).

As used herein, the term “binding partner” refers to a bioactive agent which serves as either a substrate for the CAT2 or ARG1 protein, or alternatively, as a ligand having binding affinity to the CAT2 or ARG1 protein. As mentioned above, the bioactive agent may be any of a variety of naturally-occurring or synthetic compounds, amino acids, polypeptides, polysaccharides, nucleotides or polynucleotides.

### Screening for Inhibitors of the CAT2 or ARG1 Protein

The invention provides methods of screening test compounds for inhibitors of the CAT2 or ARG1 protein, and of screening for the pharmaceutical compositions comprising the test compounds. The method of screening comprises contacting aliquots of CAT2 or ARG1 expressing cell samples with one of a plurality of test compounds, and comparing the expression of CAT2 in each of the aliquots to determine whether any of the test compounds provides a substantially decreased level of expression or activity of CAT2 or ARG1 relative to samples with other test compounds or relative to an untreated sample or control sample. In addition, methods of screening may be devised by combining a test compound with the CAT2 or ARG1 protein and thereby determining the effect of the test compound on the CAT2 or ARG1 protein.

In addition, the invention is further directed to a method of screening for test compounds capable of modulating the binding of CAT2 or ARG1 protein to a binding partner, by combining the test compound, the CAT2 or ARG1 protein, and binding partner together and determining whether binding of the binding partner and the CAT2 or ARG1 protein occurs. The test compound may be either small molecules or a biomolecule. As discussed below, test compounds may be provided from a variety of libraries well known in the art.

Other methods and compositions for screening for protein inhibitors are also known in the art (see U.S. Pat. Nos. 4,980,281, 5,266,464, 5,688,635, and 5,877,007) which are incorporated herein by reference).

Inhibitors of CAT2 or ARG1 expression, activity or binding ability are useful as therapeutic compositions of the invention. One of the inhibitors for CAT2-mediated arginine transport is lysine. Such inhibitors may be formulated as pharmaceutical compositions, as described herein below.

### High-Throughput Screening Assays

The invention provides methods of conducting high-throughput screening for test compounds capable of inhibiting the activity or expression of CAT2 or ARG1. In one embodiment, the method of high-throughput screening involves combining test compounds and the CAT2 or ARG1 protein and detecting the effect of the test compound on the CAT2 or ARG1 protein. Functional assays such as cytosensor microphysiometer, calcium flux assays such as FLIPR® (Molecular Devices Corp, Sunnyvale, Calif.), or the TUNEL assay may be employed to measure cellular activity, as discussed below.

A variety of high-throughput functional assays well known in the art may be used in combination to screen and/or study the reactivity of different types of activating test compounds. Since the coupling system is often difficult to predict, a number of assays may need to be configured to detect a wide range of coupling mechanisms. A variety of fluorescence-based techniques are well-known in the art and are capable of high-throughput and ultra-high throughput screening for activity, including but not limited to BRET® or FRET® (both by Packard Instrument Co., Meriden, Conn.). The BIACORE® system may also be manipulated to detect binding of test compounds with individual components of the therapeutic target.

By combining test compounds with the CAT2 or ARG1 protein and determining the binding activity between them, diagnostic analysis can be performed to elucidate the coupling systems. Generic assays using a cytosensor microphysiometer may also be used to measure metabolic activation, while changes in calcium mobilization can be detected by using the fluorescence-based techniques such as FLIPR® (Molecular Devices Corp, Sunnyvale, Calif.). In addition, the presence of apoptotic cells may be determined by the TUNEL assay, which utilizes flow cytometry to detect free 3-OH termini resulting from cleavage of genomic DNA during apoptosis. As mentioned above, a variety of functional assays well known in the art may be used in combination to screen and/or study the reactivity of different types of activating test compounds. In one embodiment, the high-throughput screening assay of the present invention utilizes label-free plasmon resonance technology as provided by the BIACORE® systems (Biacore International AB, Uppsala, Sweden). Plasmon free resonance occurs when surface plasmon waves are excited at a metal/liquid interface. By reflecting directed light from the surface as a result of contact with a sample, the surface plasmon resonance causes a change in the refractive index at the surface layer. The refractive index change for a given change of mass concentration at the surface layer is similar for many bioactive agents (including proteins, peptides, lipids and polynucleotides), and since the BIACORE® sensor surface can be functionalized to bind a variety of these bioactive agents, detection of a wide selection of test compounds can thus be accomplished.

Therefore, the invention provides for high-throughput screening of test compounds for the ability to inhibit an activity of the CAT2 or ARG1 protein, by combining the test compounds and the CAT2 or ARG1 protein in high-throughput assays such as BIACORE®, or in fluorescence-based assays such as BRET®. In addition, high-throughput assays may be utilized to identify specific factors that bind to the CAT2 or ARG1 protein, or alternatively, to identify test compounds which prevent binding of the CAT2 or ARG1 protein to the binding partner. Moreover, the high-throughput screening assays may be modified to determine whether test compounds can bind to either the CAT2 or ARG1 protein or to a binding partner of the CAT2 or ARG1 protein.

### Diagnostic Assays

An exemplary method for detecting the presence of CAT2 or ARG1 or polynucleotide encoding CAT2 or ARG1 in a biological sample involves obtaining a biological sample from a test subject and contacting the biological sample with a compound or an agent capable of detecting the protein or polynucleotide (e.g., mRNA, genomic DNA) that encodes CAT2 or ARG1 such that the presence of CAT2 or ARG1 polynucleotide is detected in the biological sample. An example agent for detecting mRNA or genomic DNA corresponding to the CAT2 or ARG1 gene or CAT2 or ARG1 protein is a labeled polynucleotide probe capable of hybridizing to an CAT2 or ARG1 mRNA or a genomic DNA. Suitable probes for use in the diagnostic assays of the invention are described herein. An example agent for detecting CAT2 or ARG1 proteins is an antibody which specifically recognizes CAT2 or ARG1 proteins.

The diagnostic assays may also be used to quantify the amount of expression or activity of CAT2 or ARG1 in a biological sample. Such quantification is useful, for example, to determine the progression or seventy of an inflamatory disease such as asthma, COPD, and arthritis. Such quantification is also useful, for example, to determine the severity of the inflammatory disease following treatment.

The methods described herein may be performed, for example, by utilizing prepackaged diagnostic kits comprising at least one probe polynucleotide or antibody reagent described herein, which may be conveniently used, e.g., in clinical settings to diagnose subjects exhibiting symptoms or family history of an inflammatory disease such as asthma, COPD, and arthritis.

Furthermore, any cell type or tissue in which CAT2 or ARG1 is expressed may be utilized in the prognostic or diagnostic assays described herein.

### Determining Severity of an Inflammatory Disease

In the field of diagnostic assays, the invention also provides methods for determining the severity of an inflammatory disease such as asthma, COPD, and arthritis by isolating a sample from a subject, detecting the presence, quantity and/or activity of CAT2 or ARG1 in the sample relative to a second sample from a normal sample or control sample.

In one embodiment, the expression levels of CAT2 or ARG1 in the two samples are compared, and an increased CAT2 or ARG1 expression in the test sample indicates an inflammatory disease such as asthma, COPD, and arthritis.

A example agent for detecting CAT2 or ARG1 is an antibody capable of binding to CAT2 or ARG1. In some cases, the antibody can be coupled, either directly or indirectly, to a detectable label. Antibodies can be polyclonal or monoclonal. An intact antibody, or a fragment thereof (e.g., Fab or F(ab′)₂) can be used. The term “labeled,” with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect CAT2 or ARG1 mRNA, protein or genomic DNA in a biological sample in vitro as well as in vivo. For example, in vitro techniques for detection of CAT2 or ARG1 mRNA include Northern hybridizations and in situ hybridizations. In vitro techniques for detection of CAT2 or ARG1 include enzyme linked immunosorbent assays (ELISAs), Western blots, immunoprecipitations and immunofluorescence. In vitro techniques for detection of CAT2 or ARG1 genomic DNA include Southern hybridizations. Furthermore, in vivo techniques for detection of CAT2 or ARG1 include introducing into a subject a labeled anti-CAT2 or ARG1 antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. In one example, the biological sample is a tissue sample isolated by conventional means from a subject, e.g., a biopsy.

### Prognostic Assays

The diagnostic method described herein can furthermore be utilized to identify subjects having or at risk of developing an inflammatory disease, such as asthma, COPD, and arthritis, that is associated with aberrant CAT2 or ARG1 expression or activity.

Furthermore, the prognostic assays described herein can be used to determine whether a subject can be administered an agent (e.g., an agonist, antagonist, peptidomimetic, protein, peptide, polynucleotide, small molecule, or other drug candidate) to treat or prevent an inflammatory disease associated with aberrant CAT2 or ARG1 expression or activity. Thus, the present invention provides methods for determining whether a subject can be effectively treated with an agent for an inflammatory disease associated with increased CAT2 or ARG1 expression or activity.

Prognostic assays can be devised to determine whether a subject undergoing treatment for an inflammatory disease has a poor outlook for long term survival or disease progression. In one embodiment, prognosis can be determined shortly after diagnosis, i.e., within a few days. By establishing CAT2 or ARG1 expression profiles of different stages of the inflammatory disease, from onset to later stages, an expression pattern may emerge to correlate a particular expression profile to increased likelihood of a poor prognosis. The prognosis may then be used to devise a more aggressive treatment program and enhance the likelihood of long-term survival and well-being.

### Detection of Genetic Alterations

The methods of the invention can also be used to detect genetic alterations in the CAT2 or ARG1 gene, thereby determining if a subject with the altered gene is at risk for damage characterized by aberrant regulation in CAT2 or ARG1 activity or polynucleotide expression. In many embodiments, the methods include detecting, in a sample of cells from the subject, the presence or absence of a genetic alteration characterized by at least one alteration affecting the integrity of the CAT2 or ARG1 gene, or the aberrant expression of the CAT2 or ARG1 gene. For example, such genetic alterations can be detected by ascertaining the existence of at least one of the following: 1) deletion of one or more nucleotides from the CAT2 or ARG1 gene; 2) addition of one or more nucleotides to the CAT2 or ARG1 gene; 3) substitution of one or more nucleotides of the CAT2 or ARG1 gene; 4) a chromosomal rearrangement of the CAT2 or ARG1 gene; 5) alteration in the level of a messenger RNA transcript of the CAT2 or ARG1 gene; 6) aberrant modification of the CAT2 or ARG1 gene, such as of the methylation pattern of the genomic DNA; 7) the presence of a non-wild-type splicing pattern of a messenger RNA transcript of the CAT2 or ARG1 gene; 8) non-wild-type level CAT2 or ARG1; 9) allelic loss of the CAT2 or ARG1 gene; and 10) inappropriate post-translational modification of CAT2 or ARG1. As described herein, there are a large number of assays known in the art, which can be used for detecting alterations in the CAT2 or ARG1 gene. A exemplary biological sample is a blood sample isolated by conventional means from a subject.

In certain embodiments, detection of the alteration involves the use of a probe/primer in a polymerase chain reaction (PCR), such as anchor PCR or RACE PCR, or, alternatively, in a ligation chain reaction (LCR), the latter of which can be used for detecting point mutations in the CAT2 or ARG1 gene. This method can include the steps of collecting a sample of cells from a subject, isolating a polynucleotide (e.g., genomic, mRNA or both) from the cells of the sample, contacting the polynucleotide sample with one or more primers which specifically hybridize to the CAT2 or ARG1 gene under conditions such that hybridization and amplification of the CAT2 or ARG1 gene (if present) occurs, and detecting the presence or absence of an amplification product, or detecting the size of the amplification product and comparing the length to a control sample. It is understood that PCR and/or LCR may be desirable to use as a preliminary amplification step in conjunction with any of the techniques used for detecting mutations described herein.

Alternative amplification methods include: self-sustained sequence replication, transcriptional amplification system, Q-Beta Replicase, or any other polynucleotide amplification method, followed by the detection of the amplified molecules using techniques well-known to those of skill in the art. These detection schemes are useful for the detection of polynucleotide molecules if such molecules are present in very low numbers.

In an alternative embodiment, mutations in the CAT2 or ARG1 gene from a sample cell can be identified by alterations in restriction enzyme cleavage patterns. For example, samples and control DNA are isolated, amplified (optionally), digested with one or more restriction endonucleases, and fragment length sizes are determined by gel electrophoresis and compared. Differences in fragment length sizes between sample and control DNA indicate mutations in the sample DNA. Moreover, the use of sequence specific ribozymes can be used to score for the presence of specific mutations by development or loss of a ribozyme cleavage site.

In other embodiments, genetic mutations in the CAT2 or ARG1 gene can be identified by hybridizing a sample and control polynucleotides, e.g., DNA or RNA, to high density arrays containing hundreds or thousands of oligonucleotides probes. For example, genetic mutations in the CAT2 or ARG1 gene can be identified in two-dimensional arrays containing light generated DNA probes. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the CAT2 or ARG1 gene and detect mutations by comparing the sequence of the sample CAT2 or ARG1 gene with the corresponding wild-type (control) sequence. It is also contemplated that any of a variety of automated sequencing procedures can be utilized when performing the diagnostic assays, including sequencing by mass spectrometry.

Other methods for detecting mutations in the CAT2 or ARG1 gene include methods in which protection from cleavage agents is used to detect mismatched bases in RNA/RNA or RNA/DNA heteroduplexes (Myers et al., Science, 230:1242, 1985). In general, the art technique of “mismatch cleavage” starts by providing heteroduplexes by hybridizing (labeled) RNA or DNA containing the wild-type CAT2 or ARG1 gene sequence with potentially mutant RNA or DNA obtained from a tissue sample. The double-stranded duplexes are treated with an agent which cleaves single-stranded regions of the duplex which will exist due to base pair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. In one embodiment, the control DNA or RNA can be labeled for detection.

In still another embodiment, the mismatch cleavage reaction employs one or more proteins that recognize mismatched base pairs in double-stranded DNA (so-called “DNA mismatch repair” enzymes) in defined systems for detecting and mapping point mutations in CAT2 or ARG1 cDNAs obtained from samples of cells. For example, the mutY enzyme of E. coli cleaves A at G/T mismatches and the thymidine DNA glycosylase from HeLa cells cleaves T at G/T mismatches. According to an exemplary embodiment, a probe based on the CAT2 or ARG1 gene sequence, e.g., a wild-type CAT2 or ARG1 gene sequence, is hybridized to cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in the CAT2 or ARG1 gene. For example, single strand conformation polymorphism (SSCP) may be used to detect differences in electrophoretic mobility between mutant and wild-type polynucleotides. Single-stranded DNA fragments of sample and control CAT2 or ARG1 polynucleotides will be denatured and allowed to renature. The secondary structure of single-stranded polynucleotides varies according to sequence. The resulting alteration in electrophoretic mobility enables the detection of even a single base change. The DNA fragments may be labeled or detected with labeled probes. The sensitivity of the assay may be enhanced by using RNA (rather than DNA) in which the secondary structure is more sensitive to a change in sequence. In one embodiment, the subject method utilizes heteroduplex analysis to separate double-stranded heteroduplex molecules on the basis of changes in electrophoretic mobility (Keen et al, Trends Genet. 7:5-7, 1991).

In yet another embodiment the movement of mutant or wild-type fragments in polyacrylamide gels containing a gradient of denaturant is assayed using denaturing gradient gel electrophoresis (DGGE). When DGGE is used as the method of analysis, DNA will be modified to insure that it does not completely denature, for example, by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner, Biophys. Chem. 265: 12753, 1987).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al., Proc. Natl. Acad. Sci. USA, 86: 6230, 1989). Such allele-specific oligonucleotides are hybridized to PCR amplified target or a number of different mutations when the oligonucleotides are attached to the hybridizing membrane and hybridized with labeled target DNA.

Alternatively, allele-specific amplification technology which depends on selective PCR amplification may be used in conjunction with the instant invention. Oligonucleotides used as primers for specific amplification may carry the mutation of interest in the center of the molecule (so that amplification depends on differential hybridization) or at the extreme 3′ end of one primer where, under appropriate conditions, mismatch can prevent or reduce polymerase extension. In addition, it may be desirable to introduce a novel restriction site in the region of the mutation to create cleavage-based detection. It is anticipated that in certain embodiments amplification may also be performed using Taq ligase for amplification. In such cases, ligation will occur only if there is a perfect match at the 3′ end of the 5′ sequence making it possible to detect the presence of a known mutation at a specific site by looking for the presence or absence of amplification.

### Monitoring Effects During Clinical Trials

Monitoring the influence of agents (e.g., drugs, small molecules, proteins, nucleotides) on the expression or activity of CAT2 or ARG1 can be applied not only in basic drug screening, but also in clinical trials. For example, the effectiveness of an agent determined by a screening assay, as described herein to decrease CAT2 or ARG1 expression, protein levels, or down-regulate CAT2 or ARG1 activity, can be monitored in clinical trials of subjects exhibiting increased CAT2 or ARG1 expression, protein levels, or up-regulated CAT2 or ARG1 activity. In such clinical trials, the expression or activity of CAT2 or ARG1 can be used as a “read-out” of the phenotype of a particular tissue.

For example, to study the effect of agents on CAT2- or ARG1-associated damage in a clinical trial, cells can be isolated and RNA prepared and analyzed for the levels of expression of CAT2 or ARG1. The levels of gene expression can be quantified by Northern blot analysis, RT-PCR, GeneChip® or Taqman analysis as described herein, or alternatively by measuring the amount of protein produced, by one of the methods as described herein, or by measuring the levels of activity of CAT2 or ARG1. In this way, the gene expression level can serve as a read-out, indicative of the physiological response of the cells to the agent. Accordingly, this response state may be determined before treatment and at various points during treatment of the individual with the agent.

In one embodiment, the present invention provides a method for monitoring the effectiveness of treatment of a subject with an agent (e.g., antagonist, peptidomimetic, protein, peptide, polynucleotide, small molecule, or other drug candidate identified by the screening assays described herein) including the steps of (i) obtaining a pre-administration sample from a subject prior to administration of the agent; (ii) detecting the level of expression of the CAT2 or ARG1 protein or mRNA in the pre-administration sample; (iii) obtaining one or more post-administration samples from the subject; (iv) detecting the level of expression or activity of the CAT2 or ARG1 protein or mRNA in the post-administration samples; (v) comparing the level of expression or activity of the CAT2 or ARG12 protein or mRNA in the pre-administration sample with the level of expression or activity of the CAT2 or ARG1 protein or mRNA the post-administration sample or samples; and (vi) altering the administration of the agent to the subject accordingly. For example, decreased administration of the agent may be desirable to increase expression or activity of CAT2 or ARG1 to higher levels than detected, i.e., to decrease the effectiveness of the agent. According to such an embodiment, CAT2 or ARG1 expression or activity may be used as an indicator of the effectiveness of an agent, even in the absence of an observable phenotypic response.

### Methods of Treatment

The present invention provides for both prophylactic and therapeutic methods of treating a subject at risk for, susceptible to or diagnosed with an inflammatory disease, such as asthma, COPD, osteoarthritis and rheumatoid arthritis. With regard to both prophylactic and therapeutic methods of treatment, such treatments may be specifically tailored or modified, based on knowledge obtained from the field of pharmacogenomics. “Pharmacogenomics,” as used herein, includes the application of genomics technologies such as gene sequencing, statistical genetics, and gene expression analysis to drugs in clinical development and on the market. More specifically, the term refers the study of how a subject's genes determine his or her response to a drug (e.g., a subject's “drug response phenotype” or “drug response genotype”). Thus, another aspect of the invention provides methods for tailoring an individual's prophylactic or therapeutic treatment with CAT2 or ARG1 modulators according to that individual's drug response. Pharmacogenomics allows a clinician or physician to target prophylactic or therapeutic treatments to subjects who will most benefit from the treatment and to avoid treatment of subjects who will experience toxic drug-related side effects.

### Prophylactic Methods

In one aspect, the invention provides a method for preventing CAT2- or ARG1-related pathogenic processes in a subject by administering to the subject an agent that modulates CAT2 or ARG1 expression or activity.

Subjects at risk for an inflammatory disease, such as asthma, which is associated with aberrant CAT2 or ARG1 expression or activity can be identified by, for example, any or a combination of diagnostic or prognostic assays as described herein.

Administration of a prophylactic agent can occur prior to the manifestation of symptoms characteristic of the increased CAT2 or ARG1 protein expression, such that the disease is prevented or, alternatively, delayed in its progression. Depending on the type of CAT2 or ARG1 aberrancy (e.g., typically a modulation outside the normal standard deviation), a CAT2 or ARG1 mutant protein, CAT2 or ARG1 protein antagonist agent, anti-CAT2 or -ARG1 antibody, or CAT2 or ARG1 antisense polynucleotide, for example, can be used for treating the subject. The appropriate agent can be determined based on screening assays described herein.

### Therapeutic Methods

Another aspect of the invention pertains to methods of modulating CAT2 or ARG1 protein expression or activity for therapeutic purposes. Accordingly, in an exemplary embodiment, the modulatory method of the invention involves contacting a cell with an agent that inhibits CAT2 or ARG1 expression or one or more of the activities of the CAT2 or ARG1 protein associated with the cell. An agent that modulates CAT2 or ARG12 expression or protein activity can be an agent as described herein, such as a polynucleotide, a polypeptide, or a polysaccharide, a naturally-occurring target molecule of the CAT2 or ARG1 protein (e.g., a CAT2 or ARG1 protein substrate or receptor), an anti-CAT2 or anti-ARG1 antibody, a CAT2 or an ARG1 protein antagonist, a peptidomimetic of a CAT2 or an ARG1 protein antagonist, or other small organic and inorganic molecule.

These modulatory methods can be performed in vivo (e.g., by administering the agent to a subject). As such, the present invention provides methods of treating an individual diagnosed with or at risk for an inflammatory disease characterized by enhanced expression or activity of CAT2 or ARG1. In one embodiment, the method involves administering an agent (e.g., an agent identified by a screening assay described herein) or combination of agents that down-regulates CAT2 or ARG1 expression or activity. The treatment may further be localized to the tissues or cells affected by the inflammatory disease.

### Pharmacogenomics

In conjunction with treatment for inflammatory diseases, such as asthma, COPD, and arthritis, using a CAT2 or ARG1 modulator, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a CAT2 or an ARG1 modulator as well as tailoring the dosage and/or therapeutic regimen of treatment with the CAT2 or ARG1 modulator.

Pharmacogenomics deals with clinically significant hereditary variations in the response to drugs due to altered drug disposition and abnormal action in affected persons. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is hemolysis after ingestion of oxidant drugs (anti-malarials, sulfonamides, analgesics, nitrofurans) and consumption of fava beans.

One pharmacogenomics approach to identifying genes that predict drug response, known as “a genome-wide association,” relies primarily on a high-resolution map of the human genome consisting of already known gene-related sites (e.g., a “bi-allelic” gene marker map which consists of 60,000-100,000 polymorphic or variable sites on the human genome, each of which has two variants). Such a high-resolution genetic map can be compared to a map of the genome of each of a statistically substantial number of subjects taking part in a Phase II/III drug trial to identify genes associated with a particular observed drug response or side effect. Alternatively, such a high-resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process. However, the vast majority of SNPs may not be disease associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach,” can be utilized to identify genes that predict drug response. According to this method, if a gene that encodes a drug target is known (e.g. the CAT2 or ARG1 gene), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYPZC19) has provided an explanation as to why some subjects do not obtain the expected drug effects or show exaggerated drug response and serious toxicity after taking the standard and safe dose of a drug. These polymorphisms are expressed in two phenotypes in the population, the extensive metabolizer and poor metabolizer. The prevalence of poor metabolizer phenotypes is different among different populations. For example, the gene coding for CYP2D6 is highly polymorphic and several mutations have been identified in poor metabolizers, which all lead to the absence of functional CYP2D6. Poor metabolizers of CYP2D6 and CYP2C19 quite frequently experience exaggerated drug response and side effects when they receive standard doses. If a metabolite is the active therapeutic moiety, poor metabolizers show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling” can be utilized to identify genes that predict drug response. For example, the gene expression of an animal dosed with a drug (e.g., CAT2 or ARG1 expression in response to a CAT2 or an ARG1 modulator) can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with a CAT2 or an ARG1 modulator.

### Pharmaceutical Compositions

The invention is further directed to pharmaceutical compositions comprising a CAT2 or an ARG1 modulator and a pharmaceutically acceptable carrier.

As used herein the language “pharmaceutically acceptable carrier” is intended to include any and all solvents, solubilizers, fillers, stabilizers, binders, absorbents, bases, buffering agents, lubricants, controlled release vehicles, diluents, emulsifying agents, humectants, lubricants, dispersion media, coatings, antibacterial or antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary agents can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation, sublingual, bronchial, and pulmonary), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine; propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the injectable composition should be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the requited particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents, such as sodium chloride, sugars, or polyalcohols (e.g., manitol, sorbitol), can be included in the composition. Prolonged absorption of the injectable compositions can be brought about by including in the composition an agent which delays absorption, for example, aluminum monostearate and gelatin.

Sterile injectable solutions can be prepared by incorporating the active CAT2 or ARG1 modulator in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, exemplary methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Stertes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, a nebulizer, a bronchial inhaler or a nasal drop. In addition, the compounds can be in form of a liquid solution, a gel, or a dry product. Inhalation formulations may be aqueous solutions that contain, e.g., polyoxyethylene-9-lauryl ether, glycocholate, and deoxycholate. The inhalation formulations may also contain excipients such as lactose, if needed. A nebulizer may be in aqueous suspension or solution that includes carriers or excipients to adjust pH and/or tonicity.

In one embodiment, the therapeutic moieties, which may contain a bioactive compound, are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from, e.g., Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

In one embodiment, oral or parenteral compositions formulated in dosage unit form are employed for ease of administration or uniformity of dosage. Dosage unit form as used herein includes physically discrete units suited as unitary dosages for the subject to be treated; each unit contains a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices can be selected. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. In many instances, the dosage of such compounds lies within a range of circulating concentrations that includes the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The dosage regimen for administration of a pharmaceutical composition of the present invention can be determined by the attending physician based on various factors such as the type of disease, the site of pathology, the severity of disease, the patient's age, sex, and diet, the severity of inflammation, time of administration and other clinical factors.

In one embodiment, inhalative, systemic or injectable administration can be initiated at a dose which is minimally effective, and the dose will be increased over a pre-selected time course until a positive effect is observed. Subsequently, incremental increases in dosage will be made limiting to levels that produce a corresponding increase in effect while taking into account any adverse affects that may appear. The addition of other known factors to a final composition may also affect the dosage. Progress can be monitored by periodic assessment of disease progression using standard methods.

A pharmaceutical composition of the present invention can be administered in one dose or multiple doses. The doses can be administered at any desirable intervals. In one embodiment, each dose includes about 0.1 μg-100 mg, 1 μg-10 mg, 10 μg-1 mg, or 100 μg-500 μg of an active therapeutic agent. Dosages below 0.1 μg or above 100 mg can also be used. The volume of each dose can range, for example, between 0.1 ml and 5 ml, between 0.1 ml and 1 ml, or between 0.2 ml and 0.5 ml.

The pharmaceutical compositions of the present invention can be included in a container, pack, or dispenser together with instructions for administration.

### Kits

The invention also encompasses kits for detecting the presence of a CAT2 or an ARG1 gene product in a biological sample. The kit may comprise reagents for assessing expression of CAT2 or ARG1 at nucleotide or protein level. In one embodiment, the reagents may be an antibody or fragment thereof, wherein the antibody or fragment thereof specifically binds CAT2 or ARG1. For example, antibodies of interest may be prepared by methods known in the art. Optionally, the kits may comprise a polynucleotide probe wherein the probe specifically binds to a transcribed polynucleotide corresponding to the CAT2 or ARG1 gene. The kit may contain means for determining the amount of CAT2 or ARG1 protein or mRNA in the sample and means for comparing the amount of the CAT2 or ARG1 protein or mRNA in the sample with a control or standard. The compound or agent can be packaged in a suitable container. The kit can further comprise instructions for using the kit to detect the CAT2 or ARG1 protein or polynucleotide

The invention further provides kits for assessing the suitability of each of a plurality of compounds for inhibiting an inflammatory disease in a subject. Such kits include a plurality of compounds to be tested, and a reagent (i.e., antibody specific to corresponding proteins, or a probe or primer specific to corresponding polynucleotides) for assessing expression of CAT2 or ARG1.

### EXAMPLES

### Example 1

### Gene Expression Changes in Mouse Lung Associated with Allergic Reaction

Balb/c mice (6-8 weeks of age) were obtained from Jackson Laboratories. All animals used in this study were housed in an environmentally controlled, pathogen-free facility under laminar flow hoods. All experiments conformed to the principals for laboratory animal research as outlined in the Animal Welfare Act and the Department of Health, Education and Welfare (NIH) guidelines for the experimental use of animals.

Balb/c mice were immunized by an intraperitoneal (i.p.) injection of 10 μg of OVA (Sigma, St. Louis, Mo.) in 200 μl of PBS on day 0. On days 14 and 25 mice were anesthetized with a mixture of ketamine and xylazine (45 and 8 mg/kg, respectively) and challenged intratracheally with 50 μl of a 1.5% solution of OVA or an equivalent volume of PBS. Mice were injected i.p. either with 100 μl of PBS, hIgG (400 μg/ml) or sIL-13α2-Fc (400 μg/ml) on days 24, 25 and 27. Purification of hIgG was carried according to Urban et al., Immunity 8(2): 255-645, 1998. Lungs were collected and snap frozen for RNA isolation on day 28.

To identify changes in mRNA concentration dependent on IL-13 mediated signal transduction, two of the OVA-challenged mice were treated with three intraperitoneal injections of the soluble IL-13 receptor fusion protein, sIL-13Rα2-Fc, prior to and during the course of the allergic challenge. As control for the Fc-moiety of the receptor fusion protein, two of the OVA-challenged mice were similarly treated with intraperitoneal administration of hIgG. A second set of six control mice were similarly sensitized to OVA without subsequent challenge and treated on an identical time course with intratracheal administration of PBS buffer, either alone (n=2) or with intraperitoneal injection of hIgG (n=2) or sIL-13Rα2-Fc (n=2). Lung tissue for the OVA-challenged and buffer-alone control mice was harvested at 78 hr following the second pulmonary antigen challenge (day 28).

Recombinant murine IL-13 (mIL-13; 5 μg in a final volume of 50 μl) was administrated daily for three days by intratracheal instillation to naïve Balb/c mice or Stat 6 deficient mice that had been anesthetized with a mixture of ketamine and xylazine (45 and 8 mg/kg, respectively). Lungs were collected and snap frozen in dry ice at 72 hrs after the initial IL-13 administration.

Snap frozen mouse lung tissue was pulverized using liquid nitrogen chilled mortar and pestle, suspended in 6 ml 4M guanidinium isothiocyanate/0.7%-mercaptoethanol (GTC/ME) and pulse sonicated for 2 minutes. The tissue suspension was extracted twice with acid equilibrated phenol (Promega Total RNA kit) and nucleic acid precipitated with an equal volume of isopropanol. The pellet was resuspended in 0.8 ml GTC/ME, reextracted twice with an equal volume acid phenol and once chloroform. RNA was ethanol precipitated, suspended 1DEPC treated H₂O and quantified by OD₂₈₀.

cDNA was synthesized from 10 μg of total RNA using the Superscript kit (BRL) with modification described in detail previously (Byrne et al., Current Protocols in Molecular Biology, John Wiley and Sons, Inc. (New York), 2000). First strand synthesis was carried out at 50° C. to prevent mispriming from ribosomal RNA and utilized a T7 RNA polymerase promoter containing poly-T primer (T7T24) for subsequent in vitro antisense RNA (cRNA) amplification and biotin labeling. cDNA was purified using BioMag Carboxy terminated beads (Polysciences) according to manufacture's instructions, and eluted in 48 μl of 10 mM sodium acetate, pH7.8.

In vitro T7 polymerase driven transcription reactions for synthesis and biotin labeling of antisense cRNA, Qiagen Rneasy spin column purification and cRNA fragmentation were carried out as described (supra). GeneChip hybridization mixtures contained 10 μg fragmented cRNA, 0.5 mg/ml acetylated BSA, 0.1 mg/ml herring sperm DNA, in 1×MES buffer in a total volume of 200 μl as per manufacturer's instructions. Reaction mixtures were hybridized for 18 hr at 45° C. to Affymetrix Mu11KsubA and Mu11KsubB oligonucleotide arrays. The hybridization mixtures were removed and the arrays were washed and stained with Streptavidin R-phycoerythrin (Molecular Probes) using the GeneChip Fluiditics Station 400 (Affymetrix) and scanned with a Hewlett Packard GeneArray Scanner following Manufacture's instructions. Fluorescent data was collected and converted to gene specific difference averages using MicroArray Suite 4.0 software.

An eleven member standard curve, comprised of gene fragments derived from cloned bacterial and bacteriophage sequences were spiked into each hybridization mixture at concentrations ranging from 0.5 pM to 150 pM representing RNA frequencies of approximately 3.3 to 1000 parts per million (ppm) assuming an average transcript size of 2 kb. The biotinylated standard curve fragments were synthesized by T7-polymerase driven IVT reaction from plasmid-based templates (supra). The spiked biotinylated RNA fragments serve both as an internal standard to assess chip sensitivity and as standard curve to convert measured fluorescence difference averages from individual genes into RNA frequencies in ppm. Average fluorescence difference between perfect match and single mismatch probe sets containing gene-specific oligonucleotides were used to determine frequency values with respect to the spiked standard curve. In addition, a second set of algorithms based primarily on the fraction of individual positive or negative responding probe pairs, is used to assess the absolute presence or absence of the gene product (Lockhart et al., Nat. Biotechnol. 14:1850-1856, 1996). The sensitivity of the individual microarray chip is set at one-half the minimum concentration at which 2 of any 3 adjacent standard curve spike-in templates are called present. The standard curve linear regression is forced through zero and the minimum reported gene frequency is set to the sensitivity of the individual GenChip®.

Multiple independent replicas for each of the treatment or control experimental conditions were measured and the expression data subjected to routine statistical analysis in an effort to remove false positives. Frequency values determined from individual measurements for a given experimental set were initially compared using Excel software. Average values for treatment and control animals were compared to obtain average fold change (AFC). Two-tailed Student T-tests were calculated using either unequal covariance with raw frequency values or equal covariance with log-transformed frequency values. In this work, only those genes which vary in AFC greater than 2-fold coupled to a Student t-test P<0.05 in at least one of the experimental conditions are reported. The genes sets established by the dual AFC>2-fold and t-test P<0.05 criteria were subsequently edited to remove genes called absent in the majority of test files and to remove redundancy due to genes tiled multiple times on the Mu11KsubA and subB oligonucleotide arrays. Finally, genes for which the average expression frequency of the treated animals was less than 2-fold higher than the average of inter-experimental buffer alone controls were eliminated.

The murine 11K subA and subB GeneChip® allowed the interrogation of over 13,000 murine genes, ESTs, and control sequences. The oligonucleotide arrays responded with an average sensitivity of 13 ppm and 12 ppm for the Mu11KsubA and subB oligonucleotide arrays, respectively. The quality of the purified RNA and derived cDNA product was monitored by comparing the ratio of frequencies calculated for actin and glyceraldehydes-3-phosphate dehydrogenase derived from the independent probe sets representing the 5′-end versus those from the 3′-end of the respective genes. The measured 5′/3′ ratio for RNA isolated from the different sets of control and treated animals were balanced, averaging 0.81 with a range of 0.77 and 0.90 as reported in Table 3. Of the total 13,179 tiled sequences on the combined Mu11KsubA and subB GeneChip®, an average of 5294 (+/−533) genes were called present in the individual analyzed files (Table 3) with an overall coefficient of variance of 10.1%. Additionally, the sum of computed frequencies for all genes called present in at least one file are reported for each of the subgroups, averaging 485 thousand with an overall coefficient variance of 20.9%. The similarity of chip sensitivity and RNA quality of the individual GeneChip® experiments were reflected in an overall balance in measured gene expression providing support for the use of a common spiked standard curve to normalize the individual files (Hill, A. A. et al., Genome Biol. 2(12), 2001).

The overall gene expression measured for each of the three treatment groups used to identify allergen-challenge induced gene expression (as shown in Table 3) was well balanced with respect to mRNA integrity, number of genes called present and total mRNA frequency computed across the various control and treatment files.

TABLE 3
Summary of RNA Balance

| | Balb/C 72 hr (IL-13) | | STAT6 72 hr (IL-13) | | Balb/C 72 hr (OVA) | | |
|---|---|---|---|---|---|---|---|
| | Control | IL-13 | Control | IL-13 | Control | OVA | OVA + xFc13Ra2 |
| | N = 5 | N = 6 | N = 4 | N = 5 | N = 6 | N = 4 | N = 2 |
| Avg RNA | 0.90 | 0.77 | 0.88 | 0.80 | 0.76 | 0.78 | 0.80 |
| 5′/3′Ratio | | | | | | | |
| # Genes | 5396 +/− 813 | 4984 +/− 568 | 5553 +/− 475 | 5422 +/− 5143 | 5143 +/− 285 | 5352 +/− 568 | 5210 +/− 394 |
| Present | | | | | | | |
| Frequency | 450 +/− 101 | 581 +/− 131 | 482 +/− 80 | 539 +/− 174 | 447 +/− 83 | 414 +/− 52 | 480 +/− 89 |
| Total¹ | | | | | | | |

¹Average total frequency of all genes call Present in at least 1 file (×10⁻³).

The gene expression profile measured for control mice treated with PBS was not significantly altered by intraperitoneal co-administration of human IgG or sIL-13Rα2-Fc, and thus frequency values from the six control mice were combined as a single set in the calculation of average untreated baseline expression values. Similarly, the four OVA-challenged mice treated either with intraperitoneal co-administration of buffer or hIgG were combined as a single set in calculation of average frequency values for pulmonary allergen-challenged mRNA frequency. Comparison of the average lung mRNA frequencies between the six PBS-treated control mice and four OVA-challenged mice, identified 246 tiled sequences in which the AFC was 2-fold or greater. Of this set of genes, 132 met the second selection criteria of P<0.05 and are shown in Table 4 below.

TABLE 4
Summary of Gene Expression Changes

| | Balb/C 72 hr | STAT6 72 hr | Balb/C 72 hr | PBS STAT6 |
|---|---|---|---|---|
| | IL-13 v. PBS | IL-13 v. PBS | OVA v. PBS | v. Balb/C |
| # Genes Present * | 5306 +/− 615 | 5480 +/− 536 | 5224 +/− 386 | 5472 +/− 622 |
| Total Frequency ^{†} | 522 +/− 130 | 514 +/− 136 | 441 +/− 72 | 466 +/− 86 |
| >2X AFC ^{‡} | 279 | 28 | 246 | 43 |
| P < 0.05 ^{§} | 288 | 5 | 344 | 54 |
| 2X AFC + P < 0.05 | 136 | 0 | 132 | 1 |

* Total number of mRNA transcripts called present of the 13,179 tile sequences.
^{†} Average total frequency of all genes called Present in at least 1 file (×10⁻³)
^{‡} Number of genes meeting criteria of 2-fold or greater Average Fold Change
^{§} Number of genes meeting Student t-test criteria of P < 0.05 [symbol]
Number of genes meeting dual criteria of 2-fold AFC and Student t-test P < 0.05

This allergen-induced gene set was subsequently filtered to remove genes that were called absent in a majority of the test measurements as well as several genes which are tiled redundantly oligonucleotide arrays. Average mRNA frequency values are reported for the buffer alone control mice, OVA-challenged mice and OVA-challenged mice co-administered the IL-13 antagonist. The genes were sorted by functional annotation with the relative AFC between OVA-induced and control lung expression designated by background color. It was found that the pulmonary allergic response up-regulates the expression of a diverse set of genes with only three statistically significant decreases. Many of the members of the induced allergic reaction gene set are from related functional families including Fe receptors, proteases, protease inhibitors, complement, chitinase-related proteins, immunoglobulins, and several secreted signaling proteins including chemokines and trefoil factors. Several of the genes and gene families can be linked to asthma pathobiology of epithelial cell metaplasia and mucus hypersecretion, eosinophilia, airway remodeling and airway hyperactivity (AHR).

Physiological studies demonstrated an inhibition of pulmonary eosinophil infiltration, mucous overproduction and AHR elicited by OVA challenge in mice harboring the Stat6−/− null allele (Kuperman, D. et al., J. Exp. Med. 187: 939-948, 1998; Akimoto, T. et al., J. Exp. Med. 187: 1537-1542, 1998; Miyata, S. et al., Clin. Exp. Allergy 29: 114-123, 1999). The Stat6−/− null allele was backcrossed into the Balb/C genetic background and treated with lung instillation of either mIL-13 (n=5) or PBS buffer control (n=4) using an identical protocol and schedule as for the Balb/C wildtype mice. Expression profiling of the lung tissue following multiple dose mIL-13 lung instillation identified 28 genes with an AFC ranging from 2 to 3.2-fold in the Stat6−/− mice, yet none of these genes met the T-test criteria (p<0.05) and cannot be considered statistically significant (Table 4). Additionally, none of these genes 28 genes correspond to mlL-13 induced genes in the Balb/C wildtype background. The top 25, statistically significant genes selected by AFC in the Balb/C background were sorted by expression in OVA-challenge murine lung tissue and compared to frequency values obtained for similarly treated mice harboring the Stat−/− allele. These data demonstrate the requirement for Stat function for all of the measured mIL-13-mediated gene induction in the Balb/C wildtype, consistent with the lack of physiological response to the allergen challenge in the Stat−/− null mice.

As a control, the measured lung gene expression in PBS-buffered treated Balb/C wildtype (n=4) was compared to buffer-alone treated mice harboring the Stat−/− null allele (n=4). The comparison identified 43 genes with an AFC of 2-fold or greater and 54 genes with Student T-test P<0.05 (Table 4). In these gene frequency comparisons, however, only a single gene met the dual selection criteria. The serum albumin D-box binding protein was decreased 3.2-fold in the Stat−/− null mice (P=0.03). With the single exception of the albumin D-box binding protein, these data demonstrated that in the absence of immune stimulation, there was very limited difference in overall gene expression in the mouse lung resulting from the Stat−/− null allele. In addition to the comparison of Stat−/− mIL-13 treated and buffer control mice, these results further suggested that the dual AFC and statistical criteria used to filter the data is effective in eliminating false positive calls.

### Example 2

### Gene Expression Changes in Mouse Lung Induced by IL-13 Lung Instillation

To identify IL-13 mediated changes in pulmonary gene expression, six Balb/c mice (Jackson Laboratories, Bar Harbor, Me.) were treated with multiple 5 μg dose (0 hr, 24 hr, and 48 hr) lung instillation of recombinant mouse IL-13 (mIL-13). A second set of control Balb/C mice (n=4) were instilled with buffer alone on an identical schedule. Additionally, a set of Stat6−/− null mice were treated identically with multiple dose mIL-13 (n=4) or PBS buffer (n=5) lung instillation prior to harvesting of all lungs at 78 hr for expression profiling. Comparison of gene expression profile data of Balb/c mice treated by intratracheal instillation of mIL-13 to buffer alone controls identified 279 genes with an average fold difference greater than 2-fold within the average 5306 genes called present in the individual files. Of these 279 genes, 136 met the second criteria of Student t-test P<0.05 (Table 4 above).

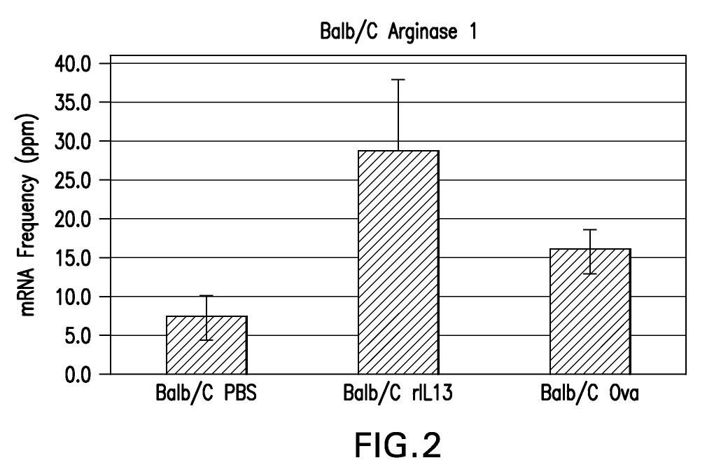

There was a dramatic overlap in gene expression mediated by the allergen challenge and direct IL-13 instillation. The observation of IL-13 up-regulated genes not identified in the OVA-induced model most likely reflects a difference in strength of signal provided by direct instillation of the cytokine. As either intratracheal IL-13 administration or lung-specific transgenic overexpression of IL-13 result in all of the pathophysiological responses seen in the mouse model of allergic asthma, the IL-13 modulated genes most likely reflect an extended set of disease-related genes. FIG. 1 shows that CAT2 and ARG1 gene are up-regulated in the Balb/c mice receiving OVA or IL-13 treatment. FIG. 2 shows the mRNA frequency of ARG1 in OVA or IL-13-treated Balb/c mice.

### Example 3

### Induction of Arg1 Gene by Ova or Adenovirus-Mediated Expression of IL-13 in Balb/c Mice

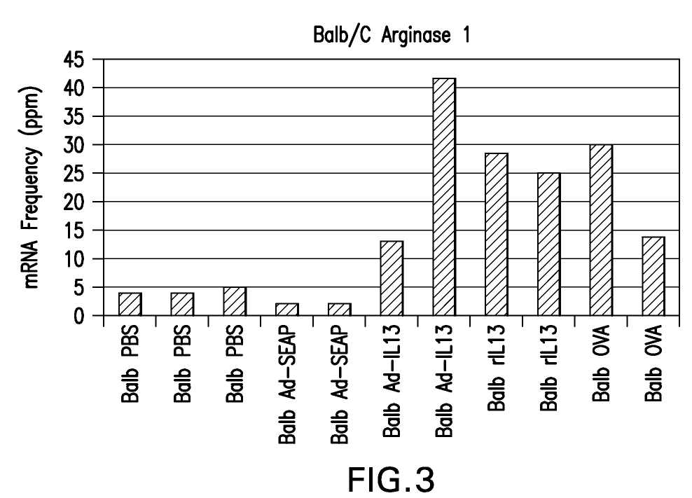

Briefly, Balb/c mice were inoculated intranasally with 5×1010 particles of a recombinant adenovirus expression murine IL-13 (Ad-IL13) or murine secreted alkaline phosphatase (Ad-SEAP). Control mice were treated with PBS, OVA, or IL-13 as described in Example 1. The animals were sacrificed 72 hours post-inoculation and the lungs were harvested for RNA extraction. RNA was prepared from the lung tissue using the RN-easy Mini kit (Qiagen) following the manufacturer's recommendations. ARG1 expression was determined using Affymetrix Mu U74Av2 oligonucleotide arrays. The results are shown in FIG. 3. The mRNA frequency is expressed as parts per million.

### Example 4

### Induction of ARG1 Gene by Adenovirus-Mediated Expression of IL-13 in C57bl/6 Mice

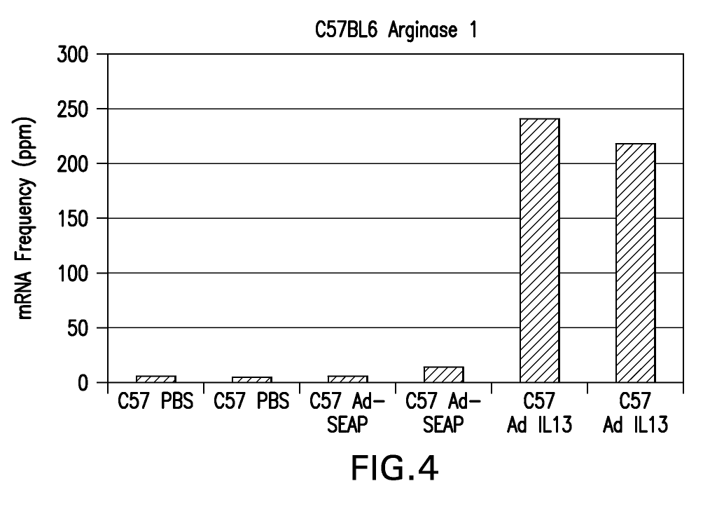

Briefly, Balb/c mice were inoculated intranasally with 5×1010 particles of Ad-IL13 or Ad-SEAP. Control mice were treated with PBS as described in Example 1. The animals were sacrificed 72 hours post-inoculation. The total lung RNA was isolated and analyzed for ARG1 expression as described in Example 2. The results are shown in FIG. 4. The mRNA frequency is expressed as parts per million.

### Example 5

### CAT2 and ARG1 Expression in Murine Macrophage Cell Line RAW264.7 Treated with LPS and IL-13

Confluent RAW264.7 cells were split 1:5 into 20 ml complete Dulbecco's modified Eagle medium (cDME) supplemented with 4 mM L-glutamine (CTS), 10% fetal bovine serum (JRH Biosciences), non-essential amino acids (Gibco), and 10 mM HEPES (Gibco). Subconfluent cells were then stimulated 24 hours later with 100 ng/ml recombinant mouse IL13 (R&D Systems) and/or 1 μg/ml lipopolysaccharide (LPS) from Pseudomonas aeruginosa Serotype 10 (Sigma). Following 24 hours of stimulation, cells were scraped from the flasks, washed 1 time with cold PBS, and the cell pellet lysed in 600 μl of buffer RLT (RN-easy Mini kit, Qiagen) containing 10 μl/ml β-mercaptoethanol. Lysates were stored at −80° C.

RNA was prepared from the treated RAW264.7 cells using the RN-easy Mini kit (Qiagen) following the manufacturer's recommendations. The RNA was quantitated by absorbance at 260 nm, and a 1:6 standard curve prepared from the LPS/IL-13-treated sample starting at 150 ng/reaction. All remaining samples were assayed at 50 ng/reaction for Arg1, CAT1, CAT2A, CAT2B, CAT3, and CAT4 using the TaqMan EZ RT-PCR kit (Applied Biosystems) and GAPDH mRNA expression to normalize. Primers were designed using the Primer Express software (Applied Biosystems). Input for Arg1, CAT1, CAT3 and CAT4 was the entire mRNA coding sequence from GenBank, while only CAT2A- and CAT2B-specific exons were used in the case of CAT2. Public databases were BLAST searched with primer sequences to ensure specificity. Primer and FAM-labeled/TAMRA-quenched probe oligonucleotides in the following sequences (5′->3′) were synthesized at Wyeth:

TABLE 5
Primers

| Gene | 5′ Primer | FAM Probe | 3′ Primer |
|---|---|---|---|
| CAT1 | SEQ ID NO: 1,518 | SEQ ID NO: 1,519 | SEQ ID NO: 1,520 |
| CAT2A | SEQ ID NO: 1,521 | SEQ ID NO: 1,522 | SEQ ID NO: 1,523 |
| CAT2B | SEQ ID NO: 1,524 | SEQ ID NO: 1,525 | SEQ ID NO: 1,526 |
| CAT3 | SEQ ID NO: 1,527 | SEQ ID NO: 1,528 | SEQ ID NO: 1,529 |
| CAT4 | SEQ ID NO: 1,530 | SEQ ID NO: 1,531 | SEQ ID NO: 1,532 |
| ARG1 | SEQ ID NO: 1,533 | SEQ ID NO: 1,534 | SEQ ID NO: 1,535 |

PCR amplification was performed on an ABI 7700 Sequence Detector (Applied Biosystems) using the standard 40 cycle parameters recommended in the EZ RT-PCR kit. Threshold cycle numbers were used to generate an indication of expression using the method of Fink et al. (Fink et al., Nat. Medicine, 4: 1329-1333, 1998). Real-time quantitative RT-PCR after laser-assisted cell picking.

FIG. 5 shows that CAT2A, CAT2B and ARG1 expression is marginally induced by LPS alone, but is significantly induced by the combination of LPS and IL-13.

### Example 6

### Arginine Uptake in RAW264.7 Cells Treated with LPS and IL-13

RAW264.7 cells, at 1×10⁶, were plated on 24-well tissue culture plates in 0.5 ml cDME. After adhering for 2 hours, 0.5 ml media containing LPS (Sigma) and/or rhIL13 (R&D Systems) was added for final concentrations of 1 g/ml and 10 ng/ml, respectively. After 20 hours incubation at 37° C. in an atmosphere of 5% CO₂ and 95% air, the cells were washed 3 times with Arg Wash Buffer #1 (140 mM choline chloride, 5 mM KCl, 0.9 mM CaCl₂, 1 mM MgSO₄, 5.6 mM glucose, and 25 mM HEPES, pH7.4), then an additional 4 times with Arg Transport Buffer (137 mM choline chloride, 5.4 mM KCl, 1.8 mM CaCl₂, 1.2 mM MgSO₄, 10 mM HEPES, adjusted to pH7.4). Transport buffer (0.5 ml) was added with 5 mM L-Leucine (Sigma) and 38 nM L-[2,3,4,5-³H]Arginine (Amersham) mixed with L-Arginine (Sigma) to a final concentration of either 400 μM L-Arginine or 100 μM L-Arginine and incubated for 3 minutes at ambient temperature. Non-saturable binding was quantitated by incubating a replicate well of each treatment with transport buffer containing mM L-Arginine. CAT2 blockade was performed in additional replicates by adding 20 mM L-Lysine (Sigma) to the transport buffer. Transport was stopped by washing 4× with ice-cold Arg Wash Buffer #2 (137 mM NaCl, 10 mM Tris, 10 mM HEPES, pH 7.4). Cells were lysed with 500 μl 1.0% SDS in 10 mM HEPES, pH 7.4. Protein quantitation was performed using a micro-BCA kit (Pierce) and 400 μl lysate suspended in 10 ml scintillation fluid, loaded into glass scintillation vials, and emissions counted for 1 minute. Specific Arginine Uptake was calculated as Saturable binding (CPM/mg protein 400 μM Arg)—Non-saturable binding (CPM/mg protein 5 mM Arg).

As shown in FIG. 6, arginine uptake is optimally induced by treating RAW264.7 cells with a combination of LPS and IL-13. The increased arginine uptake, however, is inhibited by lysine (FIG. 7), a the competitive inhibitor of CAT2 for arginine transport.

### Example 7

### Urea Production in RAW264.7 Cells Treated with LPS and IL-13

RAW264.7 macrophages were stimulated in 24-well plates as for arginine transport studies (above). After 20 hours of stimulation, the cells were washed three times with Arg Wash Buffer #1 and then an additional four times with Arg Transport Buffer. The cells were incubated at 37° C. for 24 hours in an atmosphere of 5% CO₂ and 95% air in Arg Transport Buffer containing 5 mM L-Leucine, 400 μM L-Arginine, +/−20 mM L-Lysine. Supernatants were clarified by centrifugation at 12,000 rpm for 10 minutes and 100 μl assayed for urea in triplicate using a UV absorbance kit method (R-Biopharma) following the manufacturer's instructions with the exception that it was performed at 1/10 scale in a 96-well assay plate with a total volume of 300 μl/well. Cells were lysed with 500 μl of 1.0% SDS in 10 mM HEPES, pH 7.4 and the protein quantitated using the Micro-BCA kit (Pierce). Urea production was expressed as μg urea/mg protein lysate.

FIG. 8 shows that the LPS/IL-13 treatment increases urea production in RAW264.7 cells. In agreement with the arginine uptake data shown in FIGS. 6 and 7, the increased urea production is inhibited lysine, the competitive inhibitor of CAT2 for arginine transport.

### Example 8

### Induction of ARG1 Expression Requires IL-4 Receptor

IL-4 receptor knockout mice (IL4R−/−) and IL-4 knockout mice (IL4−/−) were sensitized to OVA, or treated with PBS or IL-13 as described in FIG. 1. Total lung RNA was isolated and analyzed for ARG1 expression as described in Example 2. The results are shown in FIG. 10. The mRNA frequency is expressed as parts per million.

### Example 9

### Effect of Lysine on Carbachol-Induced Tracheal Contraction

Rats between 8-10 weeks of age were used for this experiment. Trachea were rapidly excised and cleaned of adherent connective tissue. Each trachea was sectioned into 3-4 mm in length and then cultured in a mixture of RPMI-1640 and DMEM (v/v) medium with vehicle, leucine (25 mM), lysine (100 mM) or both of them for 15-20 hours. Composition (mM) of the medium included 0.1 nonessential amino acids, 4% FBS, 2.0 glutamine, 0.05 β-mercaptoethanol, 100 U/ml penicillin/100 μg/ml streptomycin.

Trachea were supported longitudinally by a rod with a stainless steel pin into the base of a double-jacketed, glass organ bath filled with 15 ml of Krebs-Henseleit (K-H) solution (37° C.) of the following composition (mM): 118 NaCl; 4.7 KCl; 1.2 KH₂PO₄; 11.1 Dextrose; 1.2 MgSO₄; 2.8 CaCl₂; and 25 NaHCO₃. The solution was continuously gassed with a 5% CO₂ and 95% O₂ mixture for the duration of each experiment. The upper support was attached by a loop of silk thread to a TSD125 force transducer. Changes in tension of tracheal rings were synchronously recorded with a MP150 system (BIOPAC Systems, Inc.) and displayed on a PC computer.

Trachea treated with the drugs were washed with K-H solution at 10-min intervals×3 times. Carbachol (10⁻⁸ to 10⁻⁵ M)-response curves were constructed. Concentrations of the agents were increased only when the contractile responses to the previous concentrations had stabilized.

At the end of each experiment, all trachea were blotted on a gauze pad and weighted. Tensions were calculated as milligram tensions per milligram weight (mg/mg) and expressed as an individual percentage (%) of 10⁻⁵ M carbachol-evoked force of the trachea in the absence of the drugs. All values were expressed as Mean±SE. Student's paired t-test was used in this effect. A p value of less than 0.05 was considered significant.

As shown in FIG. 9, carbachol-induced rat tracheal contraction is inhibited by lysine.

### Example 10

### Carbachol-Induced Tracheal Contraction is Reduced by Deletion of the CAT2 Gene

CAT2 knockout mice (CAT2−/−) were treated as described in Example 9. As shown in FIG. 11, carbachol-induced tracheal contraction is also inhibited by the deletion of the CAT2 gene, further suggesting the involvement of CAT2 in the pathophysiology of inflammatory diseases.

### Example 11

### Association of Inhibition of ARG1 mRNA Expression with IL-13 Signaling Blockade

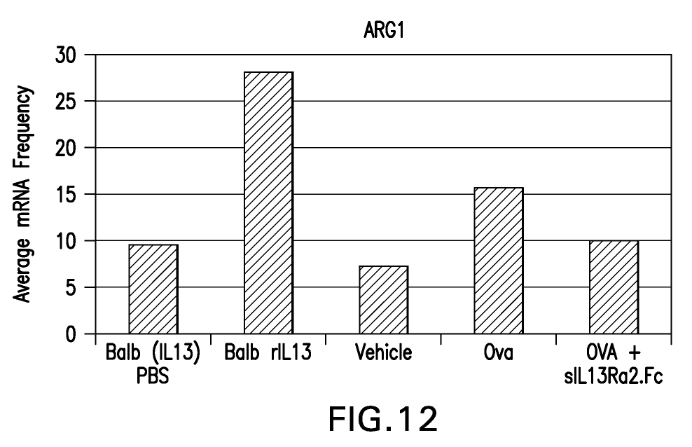

Balb/C-treated and untreated mice were sensitized to OVA, treated with PBS, rIL-13 or siL13Rα2.Fc as described in FIG. 1. Total lung RNA was isolated and analyzed for ARG1 expression as described in Example 2. The results are shown in FIG. 12. The mRNA frequency is expressed as parts per million.

While the invention has been illustrated and described in detail in the drawings and foregoing description, the same is to be considered as illustrative and not restrictive in character, it being understood that only the preferred embodiment has been shown and described and that all changes and modifications that come within the scope of the invention are desired to be protected.

What is claimed is:

1. A method comprising administering a therapeutically effective amount of an agent to a mammal which has an allergic or inflammatory disease, wherein said agent inhibits an activity or expression of a component of an arginine metabolic pathway in a tissue affected by the disease, and said component is downstream of an arginase in the pathway.

2. The method of claim 1, wherein the disease is a respiratory disease.

3. The method of claim 2, wherein the respiratory disease is asthma, chronic airway remodeling, or chronic obstructive pulmonary disease (COPD).

4. The method of claim 3, wherein said agent is capable of binding to the component or a polynucleotide encoding the component.

5.-7. (canceled)

8. The method of claim 2, wherein said agent inhibits the expression of the component by RNA interference or an antisense mechanism.

9. The method of claim 8, wherein said agent encodes or comprises an siRNA capable of inhibiting the expression of ornithine decarboxylase in said tissue by RNA interference.

10. (canceled)

11. The method of claim 2, wherein said agent is α-difluoromethylornithine.

12. (canceled)

13. The method of claim 1, wherein the mammal is a human.

14. The method of claim 13, wherein said human has asthma or COPD, and said component is an ornithine decarboxylase, and wherein said agent is capable of binding to said component or a polynucleotide encoding said component.

15.-19. (canceled)

20. A pharmaceutical composition comprising a pharmaceutically-acceptable carrier and an agent capable of inhibiting an activity or expression of a component of an arginine metabolic pathway, wherein said component is downstream of an arginase in the pathway.

21. The method of claim 1, wherein the component of an arginine metabolic pathway is selected from the group consisting of ornithine decarboxylase, S-adenosylmethionine decarboxylase, ornithine aminotransferase, ornithine transcarbamylase, spermidine synthase, and spermine synthase.

22. The method of claim 22, wherein the component of an arginine metabolic pathway is ornithine decarboxylase.

23. The composition of claim 20, wherein the component of an arginine metabolic pathway is selected from the group consisting of ornithine decarboxylase, S-adenosylmethionine decarboxylase, ornithine aminotransferase, ornithine transcarbamylase, spermidine synthase, and spermine synthase.

24. The composition of claim 23, wherein the component of an arginine metabolic pathway is ornithine decarboxylase.


SEQUENCE LISTING (1535 sequences)

SEQ ID NO: 1 - DNA, 2185 bp, Homo sapiens
  misc_feature (161)..(161): n = a, t, c,or g
gaattccggctctcaaattttctatagaatcaagatagaacctttagatgtctcaccacgaaactagcaactggaatgaagatagaaacaagtggttataactcagacaaactaatttgtcgagggtttattggaacacctgccccaccggtttgcgacanaagtttctcctgtcgccttcgtcagacgtcagaatgattccttgcagagccgcgctgacctttgcccgatgtctgatccggagaaaaatcgtgaccctggacagtctagaagacaccaaattatgccgctgcttatccaccatggacctcattgccctgggcgttggaagcacccttggggccggggtttatgtcctcgctggggaggtggccaaggcagactcgggccccagcatcgtggtgtccttcctcattgctgccctggcttcagtgatggctggcctctgctatgccgaatttggggcccgtgttcccaagacggggtctgcatatttgtacacctacgtgactgtcggagagctgtgggccttcatcactggctggaatctcattttatcgtatgtgataggtacatcaagtgttgcaagagcctggagtggcacctttgatgaacttcttagcaaacagattggtcagtttttgaggacatacttcagaatgaattacactggtcttgcagaatatcccgatttttttgctgtgtgccttatattacttctagcaggtcttttgtcttttggagtaaaagagtctgcttgggtgaataaagtcttcacagctgttaatattctcgtccttctgtttgtgatggttgctgggtttgtgaaaggaaatgtggcaaactggaagattagtgaagagtttctcaaaaatatatcagcaagtgccagagagccaccttctgaaaacggaacaagtatctatggggctggtggctttatgccttatggctttacgggaacgttggctggtgctgcaacttgcttttatgcctttgtgggatttgactgcattgcaacaactggtgaagaagttcggaatccccagaaagctattcccattggaattgtgacgtctttgcttgtttgctttatggcctattttggggtctctgcagctttaacacttatgatgccgtactacctcctcgatgaaaaaagcccccttcctgtagcgtttgaatatgtgggatggggtcctgccaaatatgtcgtcgcagctggttctctctgcgccttgtcaacaagtcttctgggctctatgtttcctttaccccgaattctgtttgccatggcccgggatggcttactgtttagatttcttgccagagtgagtaagaggcagtcaccagttgctgccacgttgactgcaggggtcatttctgctttgatggcctttctgtttgacctgaaggcgcttgtggacatgatgtccattggcacactcatggcctactctctggtggcagcctgtgttctcatcctcaggtaccagcctggcttatcttacgaccagcccaaatgttctcctgagaaagatggtctgggatcgtctcccagggtaacctcgaagagtgagtcccaggtcaccatgctgcagagacagggcttcagcatgcggaccctcttctgcccctcccttctgccaacacagcagtcagcttctctcgtgagctttctggtaggattcctagctttcctcgtgttgggcctgagtgtcttgaccacttacggagttcatgccatcaccaggctggaggcctggagcctcgctctcctcacgctgtttcttgttctcttcgttgccatcgttctcaccatctggaggcagccccagaatcagcaaaaagtagccttcatggttccattcttaccatttttgccagcgttcagcatcttggtgaacatttacttgatggtccagttaagtgcagacacttgggtcagattcagcatttggatggcaattggcttcctgatttacttttcttatggcattagacacagcctggagggtcatctgagagatgaaaacaatgaagaagatgcttatccagacaacgttcatgcagcagcagaagaaaaatctgccattcaagcaaatgaccatcacccaagaaatctcagttcacctttcatattccatgaaaagacaagtgaattctaacacttgcaggagcagat

SEQ ID NO: 2 - protein, 657 aa, Homo sapiens
MIPCRAALTFARCLIRRKIVTLDSLEDTKLCRCLSTMDLIALGVGSTLGAGVYVLAGEVAKADSGPSIVVSFLIAALASVMAGLCYAEFGARVPKTGSAYLYTYVTVGELWAFITGWNLILSYVIGTSSVARAWSGTFDELLSKQIGQFLRTYFRMNYTGLAEYPDFFAVCLILLLAGLLSFGVKESAWVNKVFTAVNILVLLFVMVAGFVKGNVANWKISEEFLKNISASAREPPSENGTSIYGAGGFMPYGFTGTLAGAATCFYAFVGFDCIATTGEEVRNPQKAIPIGIVTSLLVCFMAYFGVSAALTLMMPYYLLDEKSPLPVAFEYVGWGPAKYVVAAGSLCALSTSLLGSMFPLPRILFAMARDGLLFRFLARVSKRQSPVAATLTAGVISALMAFLFDLKALVDMMSIGTLMAYSLVAACVLILRYQPGLSYDQPKCSPEKDGLGSSPRVTSKSESQVTMLQRQGFSMRTLFCPSLLPTQQSASLVSFLVGFLAFLVLGLSVLTTYGVHAITRLEAWSLALLTLFLVLFVAIVLTIWRQPQNQQKVAFMVPFLPFLPAFSILVNIYLMVQLSADTWVRFSIWMAIGFLIYFSYGIRHSLEGHLRDENNEEDAYPDNVHAAAEEKSAIQANDHHPRNLSSPFIFHEKTSEF

SEQ ID NO: 3 - DNA, 3698 bp, Mus musculus
ctctaccaatagtttccctacgatgttagagcccaagtctcctaaggccaaggacagggggcgagacttgcaggcagcaatctcctgaggttgtgttactgttgatgggtatgaggaagttgaagggagcaacaccccacaccagcaggtcattgtacaggtgtgcagccagtgaagtacaagactgacacacaacacactccacccctcctccctgactgccttcagtctgctgaactgtgtgtccctgtagctaacaccaaatagaccatttaaatttacagtatttcttttgaacttaggttgtacatattccttggaattaatgtggagatagatagtaataataatatgtgttgatgaaatacatagaagtaagaaattaactcaccatcaaattcttaagcctcctgggtgctctgaaccaagtatctattaaagagaggcccactgcccttctgtgttcaacgggaggggagaaggcctcaaccccctcccctcccccctctcctacaggttgctctcttccaccatgattccctgcagagcagtgctgactttcgcgcgatgtctgatccggagaaaaattgtcacactggacagccttgaagattccaaactctgccgctgcttaaccaccgtggacctcatcgctttgggggttggaagcactctgggcgctggggtctacgtcctggctggggaagtcgccaaagccgattctggcccgagtatcgtggtgtctttcctcatcgctgccctggcctcggttatggccggcctttgctatgctgaatttggggcccgagtacccaagactggatctgcgtatctatacacttacgtcacggtcggagagctgtgggccttcatcactggctggaatctcatcctgtcatatgtcataggtacgtccagtgtcgcaagagcatggagtggcacctttgacgaacttcttaataaacagattggccagtttttcaaaacgtacttcaaaatgaattacactggtctggcagagtatccagacttctttgccgtgtgccttgtattactcctggcaggtcttttatcttttggagtaaaagagtctgcttgggtgaataaattttttacagctattaatatcctggtccttctctttgtcatggtggctgggtttgtgaaaggaaatgtggctaactggaagatcagtgaagagtttctcaaaaatatatcagcaagtgctagagaaccaccttctgagaacggaacaagcatctacggggctggcggctttatgccctatggctttacagggacgttggctggtgctgcaacgtgcttttatgcctttgtgggctttgactgcattgcaacaaccggtgaagaggttcggaatccacaaaaggcgatccccatcggaatagtgacgtccttacttgtctgctttatggcttactttggggtttctgcagctttaacgcttatgatgccttactacctcctggatgagaaaagtccactcccagtcgcgtttgagtatgtcagatggggccccgccaaatacgttgtcgcagcaggctccctctgcgccttatcaacaagtcttctgggttctatgttccccttaccccgaattctgtttgccatggcccgggatggcttactgtttagatttcttgcaagagtgagtaagaggcagtcacccgttgctgccacgatgactgcaggggtcatttctgctgtgatggcctttctttttgacctgaaggccctcgtggacatgatgtctattggcaccctcatggcctactctctggtggcagcctgtgtgcttattctcaggtaccaacctggcttgtgttacgagcagcccaaatacacccctgagaaagaaactctggaatcatgtaccaatgcgactttgaagagcgagtcccaggtcaccatgctgcaaggacagggtttcagcctacgaaccctcttcagcccctctgccctgcccacacgacagtcggcttcccttgtgagctttctggtgggattcctggctttcctcatcctgggcttgagtattctaaccacgtatggcgtccaggccattgccagactggaagcctggagcctggctcttctcgccctgttccttgtcctctgcgctgccgtcattctgaccatttggaggcagccacagaatcagcaaaaagtagccttcatggtcccgttcttaccgtttctgccggccttcagcatcctggtcaacatttacttgatggtccagttaagtgcggacacttggatcagattcagcatctggatggcgcttggctttctgatctatttcgcctatggcattagacacagcttggagggtaaccccagggacgaagaagacgatgaggatgccttttcagaaaacatcaatgtagcaacagaagaaaagtccgtcatgcaagcaaatgaccatcaccaaagaaacctcagcttacctttcatacttcatgaaaagacaagtgaatgttgatgctggccctcggtcttaccacgcataccttaacaatgagtacactgtggccggatgccaccatcgtgctgggctgtcgtgggtctgctgtggacatggcttgcctaacttgtacttcctcctccagacagcttctcttcagatggtggattctgtgtctgaggagactgcctgagagcactcctcagctatatgtatccccaaaacagtatgtccgtgtgcgtacatgtatgtctgcgatgtgagtgttcaatgttgtccgttattagtctgtgacataattccagcatggtaattggtggcatatactgcacacactagtaaacagtatattgctgaatagagatgtattctgtatatgtcctaggtggctggggaaatagtggtggtttctttattaggtatatgaccatcagtttggacatactgaaatgccatcccctgtcaggatgtttaacagtggtcatgggtggggaagggataaggaatgggcattgtctataaattgtaatgcatatatccttctcctacttgctaagacagctttcttaaacggccagggagagtgtttctttcctctgtatgacaagatgaagaggtagtctgtggctggagatggccaatcctggttttgagagccttggtccttaaatataacacttctacctgctgtaaatgatatacccatcaactccttgaaagtctcaggaaaggtaccgtggcctacttgggaccctttaagccagagagtgaacgtagtcccagtggcttctaaagatcagatgacttacaaagatgcttggcagagactgaaggcagaataggcctccgaatcacacatttggtagactggttcaggctgtttcccattacttcagacctaatcacagctcagcgtctgctccttcatcacagaacccagtcttttgtttctcatctttcctttgatctggatgaaaacgagtgtggtagcaaattctttttttactgtggacaaaaaagacacagatggaggggtataggaaaatgcccttagtccagtctcacagggcgcttgagcccagttgaaggattttaaacctaattttcctctgatatttcttttagaaatgtttagagattctatctatctcctaccctatctcatcatttagcacttataaatttattttctccctgtctgtctgtcccc

SEQ ID NO: 4 - protein, 657 aa, Mus musculus
MIPCRAVLTFARCLIRRKIVTLDSLEDSKLCRCLTTVDLIALGVGSTLGAGVYVLAGEVAKADSGPSIVVSFLIAALASVMAGLCYAEFGARVPKTGSAYLYTYVTVGELWAFITGWNLILSYVIGTSSVARAWSGTFDELLNKQIGQFFKTYFKMNYTGLAEYPDFFAVCLVLLLAGLLSFGVKESAWVNKFFTAINILVLLFVMVAGFVKGNVANWKISEEFLKNISASAREPPSENGTSIYGAGGFMPYGFTGTLAGAATCFYAFVGFDCIATTGEEVRNPQKAIPIGIVTSLLVCFMAYFGVSAALTLMMPYYLLDEKSPLPVAFEYVRWGPAKYVVAAGSLCALSTSLLGSMFPLPRILFAMARDGLLFRFLARVSKRQSPVAATMTAGVISAVMAFLFDLKALVDMMSIGTLMAYSLVAACVLILRYQPGLCYEQPKYTPEKETLESCTNATLKSESQVTMLQGQGFSLRTLFSPSALPTRQSASLVSFLVGFLAFLILGLSILTTYGVQAIARLEAWSLALLALFLVLCAAVILTIWRQPQNQQKVAFMVPFLPFLPAFSILVNIYLMVQLSADTWIRFSIWMALGFLIYFAYGIRHSLEGNPRDEEDDEDAFSENINVATEEKSVMQANDHHQRNLSLPFILHEKTSEC

SEQ ID NO: 5 - DNA, 1447 bp, Homo sapiens
tgtcactgagggttgactgactggagagctcaagtgcagcaaagagaagtgtcagagcatgagcgccaagtccagaaccatagggattattggagctcctttctcaaagggacagccacgaggaggggtggaagaaggccctacagtattgagaaaggctggtctgcttgagaaacttaaagaacaagagtgtgatgtgaaggattatggggacctgccctttgctgacatccctaatgacagtccctttcaaattgtgaagaatccaaggtctgtgggaaaagcaagcgagcagctggctggcaaggtggcagaagtcaagaagaacggaagaatcagcctggtgctgggcggagaccacagtttggcaattggaagcatctctggccatgccagggtccaccctgatcttggagtcatctgggtggatgctcacactgatatcaacactccactgacaaccacaagtggaaacttgcatggacaacctgtatctttcctcctgaaggaactaaaaggaaagattcccgatgtgccaggattctcctgggtgactccctgtatatctgccaaggatattgtgtatattggcttgagagacgtggaccctggggaacactacattttgaaaactctaggcattaaatacttttcaatgactgaagtggacagactaggaattggcaaggtgatggaagaaacactcagctatctactaggaagaaagaaaaggccaattcatctaagttttgatgttgacggactggacccatctttcacaccagctactggcacaccagtcgtgggaggtctgacatacagagaaggtctctacatcacagaagaaatctacaaaacagggctactctcaggattagatataatggaagtgaacccatccctggggaagacaccagaagaagtaactcgaacagtgaacacagcagttgcaataaccttggcttgtttcggacttgctcgggagggtaatcacaagcctattgactaccttaacccacctaagtaaatgtggaaacatccgatataaatctcatagttaatggcataattagaaagctaatcattttcttaagcatagagttatccttctaaagacttgttctttcagaaaaatgtttttccaattagtataaactctacaaattccctcttggtgtaaaattcaagatgtggaaattctaacttttttgaaatttaaaagcttatattttctaacttggcaaaagacttatccttagaaagagaagtgtacattgatttccaattaaaaatttgctggcattaaaaataagcacacttacataagcccccatacatagagtgggactcttggaatcaggagacaaagctaccacatgtggaaaggtactatgtgtccatgtcattcaaaaaatgtgattttttataataaactctttataacaag

SEQ ID NO: 6 - protein, 322 aa, Homo sapiens
MSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPK

SEQ ID NO: 7 - DNA, 1221 bp, Mus musculus
ctacaagggcaagaaatacaagcccctagacctgcgacccaagaagactagagccatgcgccgccggctcaccaagcacgaggaggagctggacagcccgagcacatgcagcagcagcagccgctggaacccagagagagcatgagctccaagccaaagtccttagagattatcggagcgcctttctcaaaaggacagcctcgaggaggggtagagaaaggccctgcagcactgaggaaagctggtctgctggaaaaacttaaagaaacagagtatgacgtgagagaccacggggacctggcctttgttgatgtccctaatgacagctcctttcaaattgtgaagaacccacggtctgtggggaaagccaatgaagagctggctggtgtggtggcagaggtccagaagaatggaagagtcagtgtggtgctgggtggagaccacagtctggcagttggaagcatctctggccacgccagggtccaccctgacctatgtgtcatttgggtggatgctcacactgacatcaacactcccctgacaaccagctctgggaatctgcatgggcaacctgtgtcctttctcctgaaggaactgaaaggaaagttcccagatgtaccaggattctcctgggtgactccctgcatatctgccaaagacatcgtgtacattggcttgcgagacgtagaccctggggaacactatataataaaaactctgggaattaagtatttctccatgactgaagtagacaagctggggattggcaaggtgatggaagagaccttcagctacctgctgggaaggaagaaaaggccgattcacctgagctttgatgtcgacgggctggacccagcattcaccccggcgaccggcaccccggttctgggaggcctatcttacagagaaggtctctacatcacagaagaaatttacaagacagggctcctttcaggactagatatcatggaagtgaacccaactcttgggaagacagcagaggaggtgaagagtactgtgaacacggcagtggctttaaccttggcttgcttcggaactcaacgggagggtaaccataagccagggactgactaccttaaaccacctaagtgactgtgaatgcgccacatgaaaaccatctggggcatcacagcaaagcagacagaactaagcaaacgccttctcctcccaagggcttgttcttttaaaaaaaaaaaaaaa

SEQ ID NO: 8 - protein, 323 aa, Mus musculus
MSSKPKSLEIIGAPFSKGQPRGGVEKGPAALRKAGLLEKLKETEYDVRDHGDLAFVDVPNDSSFQIVKNPRSVGKANEELAGVVAEVQKNGRVSVVLGGDHSLAVGSISGHARVHPDLCVIWVDAHTDINTPLTTSSGNLHGQPVSFLLKELKGKFPDVPGFSWVTPCISAKDIVYIGLRDVDPGEHYIIKTLGIKYFSMTEVDKLGIGKVMEETFSYLLGRKKRPIHLSFDVDGLDPAFTPATGTPVLGGLSYREGLYITEEIYKTGLLSGLDIMEVNPTLGKTAEEVKSTVNTAVALTLACFGTQREGNHKPGTDYLKPPK

SEQ ID NO: 9 - DNA, 21 bp, Homo sapiens
aagatagaacctttagatgtc

SEQ ID NO: 10 - RNA, 21 nt, RNAi-sense strand
gauagaaccuuuagaugucuu

SEQ ID NO: 11 - RNA, 21 nt, RNAi-antisense strand
gacaucuaaagguucuaucuu

SEQ ID NO: 12 - DNA, 21 bp, Homo sapiens
aacctttagatgtctcaccac

SEQ ID NO: 13 - RNA, 21 nt, RNAi-sense strand
ccuuuagaugucucaccacuu

SEQ ID NO: 14 - RNA, 21 nt, RNAi-antisense strand
guggugagacaucuaaagguu

SEQ ID NO: 15 - DNA, 21 bp, Homo sapiens
aaactagcaactggaatgaag

SEQ ID NO: 16 - RNA, 21 nt, RNAi-sense strand
acuagcaacuggaaugaaguu

SEQ ID NO: 17 - RNA, 21 nt, RNAi-antisense strand
cuucauuccaguugcuaguuu

SEQ ID NO: 18 - DNA, 21 bp, Homo sapiens
aactagcaactggaatgaaga

SEQ ID NO: 19 - RNA, 21 nt, RNAi-sense strand
cuagcaacuggaaugaagauu

SEQ ID NO: 20 - RNA, 21 nt, RNAi-antisense strand
ucuucauuccaguugcuaguu

SEQ ID NO: 21 - DNA, 21 bp, Homo sapiens
aactggaatgaagatagaaac

SEQ ID NO: 22 - RNA, 21 nt, RNAi-sense strand
cuggaaugaagauagaaacuu

SEQ ID NO: 23 - RNA, 21 nt, RNAi-antisense strand
guuucuaucuucauuccaguu

SEQ ID NO: 24 - DNA, 21 bp, Homo sapiens
aatgaagatagaaacaagtgg

SEQ ID NO: 25 - RNA, 21 nt, RNAi-sense strand
ugaagauagaaacaagugguu

SEQ ID NO: 26 - RNA, 21 nt, RNAi-antisense strand
ccacuuguuucuaucuucauu

SEQ ID NO: 27 - DNA, 21 bp, Homo sapiens
aaacaagtggttataactcag

SEQ ID NO: 28 - RNA, 21 nt, RNAi-sense strand
acaagugguuauaacucaguu

SEQ ID NO: 29 - RNA, 21 nt, RNAi-antisense strand
cugaguuauaaccacuuguuu

SEQ ID NO: 30 - DNA, 21 bp, Homo sapiens
aacaagtggttataactcaga

SEQ ID NO: 31 - RNA, 21 nt, RNAi-sense strand
caagugguuauaacucagauu

SEQ ID NO: 32 - RNA, 21 nt, RNAi-antisense strand
ucugaguuauaaccacuuguu

SEQ ID NO: 33 - DNA, 21 bp, Homo sapiens
aagtggttataactcagacaa

SEQ ID NO: 34 - RNA, 21 nt, RNAi-sense strand
gugguuauaacucagacaauu

SEQ ID NO: 35 - RNA, 21 nt, RNAi-antisense strand
uugucugaguuauaaccacuu

SEQ ID NO: 36 - DNA, 21 bp, Homo sapiens
aagtttctcctgtcgccttcg

SEQ ID NO: 37 - RNA, 21 nt, RNAi-sense strand
guuucuccugucgccuucguu

SEQ ID NO: 38 - RNA, 21 nt, RNAi-antisense strand
cgaaggcgacaggagaaacuu

SEQ ID NO: 39 - DNA, 21 bp, Homo sapiens
aatgattccttgcagagccgc

SEQ ID NO: 40 - RNA, 21 nt, RNAi-sense strand
ugauuccuugcagagccgcuu

SEQ ID NO: 41 - RNA, 21 nt, RNAi-antisense strand
gcggcucugcaaggaaucauu

SEQ ID NO: 42 - DNA, 21 bp, Homo sapiens
aagacaccaaattatgccgct

SEQ ID NO: 43 - RNA, 21 nt, RNAi-sense strand
gacaccaaauuaugccgcuuu

SEQ ID NO: 44 - RNA, 21 nt, RNAi-antisense strand
agcggcauaauuuggugucuu

SEQ ID NO: 45 - DNA, 21 bp, Homo sapiens
aaattatgccgctgcttatcc

SEQ ID NO: 46 - RNA, 21 nt, RNAi-sense strand
auuaugccgcugcuuauccuu

SEQ ID NO: 47 - RNA, 21 nt, RNAi-antisense strand
ggauaagcagcggcauaauuu

SEQ ID NO: 48 - DNA, 21 bp, Homo sapiens
aattatgccgctgcttatcca

SEQ ID NO: 49 - RNA, 21 nt, RNAi-sense strand
uuaugccgcugcuuauccauu

SEQ ID NO: 50 - RNA, 21 nt, RNAi-antisense strand
uggauaagcagcggcauaauu

SEQ ID NO: 51 - DNA, 21 bp, Homo sapiens
aagtgttgcaagagcctggag

SEQ ID NO: 52 - RNA, 21 nt, RNAi-sense strand
guguugcaagagccuggaguu

SEQ ID NO: 53 - RNA, 21 nt, RNAi-antisense strand
cuccaggcucuugcaacacuu

SEQ ID NO: 54 - DNA, 21 bp, Homo sapiens
aagagcctggagtggcacctt

SEQ ID NO: 55 - RNA, 21 nt, RNAi-sense strand
gagccuggaguggcaccuuuu

SEQ ID NO: 56 - RNA, 21 nt, RNAi-antisense strand
aaggugccacuccaggcucuu

SEQ ID NO: 57 - DNA, 21 bp, Homo sapiens
aacttcttagcaaacagattg

SEQ ID NO: 58 - RNA, 21 nt, RNAi-sense strand
cuucuuagcaaacagauuguu

SEQ ID NO: 59 - RNA, 21 nt, RNAi-antisense strand
caaucuguuugcuaagaaguu

SEQ ID NO: 60 - DNA, 21 bp, Homo sapiens
aatgaattacactggtcttgc

SEQ ID NO: 61 - RNA, 21 nt, RNAi-sense strand
ugaauuacacuggucuugcuu

SEQ ID NO: 62 - RNA, 21 nt, RNAi-antisense strand
gcaagaccaguguaauucauu

SEQ ID NO: 63 - DNA, 21 bp, Homo sapiens
aattacactggtcttgcagaa

SEQ ID NO: 64 - RNA, 21 nt, RNAi-sense strand
uuacacuggucuugcagaauu

SEQ ID NO: 65 - RNA, 21 nt, RNAi-antisense strand
uucugcaagaccaguguaauu

SEQ ID NO: 66 - DNA, 21 bp, Homo sapiens
aataaagtcttcacagctgtt

SEQ ID NO: 67 - RNA, 21 nt, RNAi-sense strand
uaaagucuucacagcuguuuu

SEQ ID NO: 68 - RNA, 21 nt, RNAi-antisense strand
aacagcugugaagacuuuauu

SEQ ID NO: 69 - DNA, 21 bp, Homo sapiens
aaagtcttcacagctgttaat

SEQ ID NO: 70 - RNA, 21 nt, RNAi-sense strand
agucuucacagcuguuaauuu

SEQ ID NO: 71 - RNA, 21 nt, RNAi-antisense strand
auuaacagcugugaagacuuu

SEQ ID NO: 72 - DNA, 21 bp, Homo sapiens
aagtcttcacagctgttaata

SEQ ID NO: 73 - RNA, 21 nt, RNAi-sense strand
gucuucacagcuguuaauauu

SEQ ID NO: 74 - RNA, 21 nt, RNAi-antisense strand
uauuaacagcugugaagacuu

SEQ ID NO: 75 - DNA, 21 bp, Homo sapiens
aatattctcgtccttctgttt

SEQ ID NO: 76 - RNA, 21 nt, RNAi-sense strand
uauucucguccuucuguuuuu

SEQ ID NO: 77 - RNA, 21 nt, RNAi-antisense strand
aaacagaaggacgagaauauu

SEQ ID NO: 78 - DNA, 21 bp, Homo sapiens
aaaggaaatgtggcaaactgg

SEQ ID NO: 79 - RNA, 21 nt, RNAi-sense strand
aggaaauguggcaaacugguu

SEQ ID NO: 80 - RNA, 21 nt, RNAi-antisense strand
ccaguuugccacauuuccuuu

SEQ ID NO: 81 - DNA, 21 bp, Homo sapiens
aaggaaatgtggcaaactgga

SEQ ID NO: 82 - RNA, 21 nt, RNAi-sense strand
ggaaauguggcaaacuggauu

SEQ ID NO: 83 - RNA, 21 nt, RNAi-antisense strand
uccaguuugccacauuuccuu

SEQ ID NO: 84 - DNA, 21 bp, Homo sapiens
aaatgtggcaaactggaagat

SEQ ID NO: 85 - RNA, 21 nt, RNAi-sense strand
auguggcaaacuggaagauuu

SEQ ID NO: 86 - RNA, 21 nt, RNAi-antisense strand
aucuuccaguuugccacauuu

SEQ ID NO: 87 - DNA, 21 bp, Homo sapiens
aatgtggcaaactggaagatt

SEQ ID NO: 88 - RNA, 21 nt, RNAi-sense strand
uguggcaaacuggaagauuuu

SEQ ID NO: 89 - RNA, 21 nt, RNAi-antisense strand
aaucuuccaguuugccacauu

SEQ ID NO: 90 - DNA, 21 bp, Homo sapiens
aaactggaagattagtgaaga

SEQ ID NO: 91 - RNA, 21 nt, RNAi-sense strand
acuggaagauuagugaagauu

SEQ ID NO: 92 - RNA, 21 nt, RNAi-antisense strand
ucuucacuaaucuuccaguuu

SEQ ID NO: 93 - DNA, 21 bp, Homo sapiens
aactggaagattagtgaagag

SEQ ID NO: 94 - RNA, 21 nt, RNAi-sense strand
cuggaagauuagugaagaguu

SEQ ID NO: 95 - RNA, 21 nt, RNAi-antisense strand
cucuucacuaaucuuccaguu

SEQ ID NO: 96 - DNA, 21 bp, Homo sapiens
aagattagtgaagagtttctc

SEQ ID NO: 97 - RNA, 21 nt, RNAi-sense strand
gauuagugaagaguuucucuu

SEQ ID NO: 98 - RNA, 21 nt, RNAi-antisense strand
gagaaacucuucacuaaucuu

SEQ ID NO: 99 - DNA, 21 bp, Homo sapiens
aaatatatcagcaagtgccag

SEQ ID NO: 100 - RNA, 21 nt, RNAi-sense strand
auauaucagcaagugccaguu

SEQ ID NO: 101 - RNA, 21 nt, RNAi-antisense strand
cuggcacuugcugauauauuu

SEQ ID NO: 102 - DNA, 21 bp, Homo sapiens
aatatatcagcaagtgccaga

SEQ ID NO: 103 - RNA, 21 nt, RNAi-sense strand
uauaucagcaagugccagauu

SEQ ID NO: 104 - RNA, 21 nt, RNAi-antisense strand
ucuggcacuugcugauauauu

SEQ ID NO: 105 - DNA, 21 bp, Homo sapiens
aagtgccagagagccaccttc

SEQ ID NO: 106 - RNA, 21 nt, RNAi-sense strand
gugccagagagccaccuucuu

SEQ ID NO: 107 - RNA, 21 nt, RNAi-antisense strand
gaagguggcucucuggcacuu

SEQ ID NO: 108 - DNA, 21 bp, Homo sapiens
aaacggaacaagtatctatgg

SEQ ID NO: 109 - RNA, 21 nt, RNAi-sense strand
acggaacaaguaucuaugguu

SEQ ID NO: 110 - RNA, 21 nt, RNAi-antisense strand
ccauagauacuuguuccguuu

SEQ ID NO: 111 - DNA, 21 bp, Homo sapiens
aacgttggctggtgctgcaac

SEQ ID NO: 112 - RNA, 21 nt, RNAi-sense strand
cguuggcuggugcugcaacuu

SEQ ID NO: 113 - RNA, 21 nt, RNAi-antisense strand
guugcagcaccagccaacguu

SEQ ID NO: 114 - DNA, 21 bp, Homo sapiens
aacaactggtgaagaagttcg

SEQ ID NO: 115 - RNA, 21 nt, RNAi-sense strand
caacuggugaagaaguucguu

SEQ ID NO: 116 - RNA, 21 nt, RNAi-antisense strand
cgaacuucuucaccaguuguu

SEQ ID NO: 117 - DNA, 21 bp, Homo sapiens
aactggtgaagaagttcggaa

SEQ ID NO: 118 - RNA, 21 nt, RNAi-sense strand
cuggugaagaaguucggaauu

SEQ ID NO: 119 - RNA, 21 nt, RNAi-antisense strand
uuccgaacuucuucaccaguu

SEQ ID NO: 120 - DNA, 21 bp, Homo sapiens
aattgtgacgtctttgcttgt

SEQ ID NO: 121 - RNA, 21 nt, RNAi-sense strand
uugugacgucuuugcuuguuu

SEQ ID NO: 122 - RNA, 21 nt, RNAi-antisense strand
acaagcaaagacgucacaauu

SEQ ID NO: 123 - DNA, 21 bp, Homo sapiens
aacacttatgatgccgtacta

SEQ ID NO: 124 - RNA, 21 nt, RNAi-sense strand
cacuuaugaugccguacuauu

SEQ ID NO: 125 - RNA, 21 nt, RNAi-antisense strand
uaguacggcaucauaaguguu

SEQ ID NO: 126 - DNA, 21 bp, Homo sapiens
aaatatgtcgtcgcagctggt

SEQ ID NO: 127 - RNA, 21 nt, RNAi-sense strand
auaugucgucgcagcugguuu

SEQ ID NO: 128 - RNA, 21 nt, RNAi-antisense strand
accagcugcgacgacauauuu

SEQ ID NO: 129 - DNA, 21 bp, Homo sapiens
aatatgtcgtcgcagctggtt

SEQ ID NO: 130 - RNA, 21 nt, RNAi-sense strand
uaugucgucgcagcugguuuu

SEQ ID NO: 131 - RNA, 21 nt, RNAi-antisense strand
aaccagcugcgacgacauauu

SEQ ID NO: 132 - DNA, 21 bp, Homo sapiens
aagaggcagtcaccagttgct

SEQ ID NO: 133 - RNA, 21 nt, RNAi-sense strand
gaggcagucaccaguugcuuu

SEQ ID NO: 134 - RNA, 21 nt, RNAi-antisense strand
agcaacuggugacugccucuu

SEQ ID NO: 135 - DNA, 21 bp, Homo sapiens
aaggcgcttgtggacatgatg

SEQ ID NO: 136 - RNA, 21 nt, RNAi-sense strand
ggcgcuuguggacaugauguu

SEQ ID NO: 137 - RNA, 21 nt, RNAi-antisense strand
caucauguccacaagcgccuu

SEQ ID NO: 138 - DNA, 21 bp, Homo sapiens
aaatgttctcctgagaaagat

SEQ ID NO: 139 - RNA, 21 nt, RNAi-sense strand
auguucuccugagaaagauuu

SEQ ID NO: 140 - RNA, 21 nt, RNAi-antisense strand
aucuuucucaggagaacauuu

SEQ ID NO: 141 - DNA, 21 bp, Homo sapiens
aatgttctcctgagaaagatg

SEQ ID NO: 142 - RNA, 21 nt, RNAi-sense strand
uguucuccugagaaagauguu

SEQ ID NO: 143 - RNA, 21 nt, RNAi-antisense strand
caucuuucucaggagaacauu

SEQ ID NO: 144 - DNA, 21 bp, Homo sapiens
aacacagcagtcagcttctct

SEQ ID NO: 145 - RNA, 21 nt, RNAi-sense strand
cacagcagucagcuucucuuu

SEQ ID NO: 146 - RNA, 21 nt, RNAi-antisense strand
agagaagcugacugcuguguu

SEQ ID NO: 147 - DNA, 21 bp, Homo sapiens
aaagtagccttcatggttcca

SEQ ID NO: 148 - RNA, 21 nt, RNAi-sense strand
aguagccuucaugguuccauu

SEQ ID NO: 149 - RNA, 21 nt, RNAi-antisense strand
uggaaccaugaaggcuacuuu

SEQ ID NO: 150 - DNA, 21 bp, Homo sapiens
aagtagccttcatggttccat

SEQ ID NO: 151 - RNA, 21 nt, RNAi-sense strand
guagccuucaugguuccauuu

SEQ ID NO: 152 - RNA, 21 nt, RNAi-antisense strand
auggaaccaugaaggcuacuu

SEQ ID NO: 153 - DNA, 21 bp, Homo sapiens
aacatttacttgatggtccag

SEQ ID NO: 154 - RNA, 21 nt, RNAi-sense strand
cauuuacuugaugguccaguu

SEQ ID NO: 155 - RNA, 21 nt, RNAi-antisense strand
cuggaccaucaaguaaauguu

SEQ ID NO: 156 - DNA, 21 bp, Homo sapiens
aattggcttcctgatttactt

SEQ ID NO: 157 - RNA, 21 nt, RNAi-sense strand
uuggcuuccugauuuacuuuu

SEQ ID NO: 158 - RNA, 21 nt, RNAi-antisense strand
aaguaaaucaggaagccaauu

SEQ ID NO: 159 - DNA, 21 bp, Homo sapiens
aatgaagaagatgcttatcca

SEQ ID NO: 160 - RNA, 21 nt, RNAi-sense strand
ugaagaagaugcuuauccauu

SEQ ID NO: 161 - RNA, 21 nt, RNAi-antisense strand
uggauaagcaucuucuucauu

SEQ ID NO: 162 - DNA, 21 bp, Homo sapiens
aagaagatgcttatccagaca

SEQ ID NO: 163 - RNA, 21 nt, RNAi-sense strand
gaagaugcuuauccagacauu

SEQ ID NO: 164 - RNA, 21 nt, RNAi-antisense strand
ugucuggauaagcaucuucuu

SEQ ID NO: 165 - DNA, 21 bp, Homo sapiens
aagatgcttatccagacaacg

SEQ ID NO: 166 - RNA, 21 nt, RNAi-sense strand
gaugcuuauccagacaacguu

SEQ ID NO: 167 - RNA, 21 nt, RNAi-antisense strand
cguugucuggauaagcaucuu

SEQ ID NO: 168 - DNA, 21 bp, Homo sapiens
aacgttcatgcagcagcagaa

SEQ ID NO: 169 - RNA, 21 nt, RNAi-sense strand
cguucaugcagcagcagaauu

SEQ ID NO: 170 - RNA, 21 nt, RNAi-antisense strand
uucugcugcugcaugaacguu

SEQ ID NO: 171 - DNA, 21 bp, Homo sapiens
aaatctgccattcaagcaaat

SEQ ID NO: 172 - RNA, 21 nt, RNAi-sense strand
aucugccauucaagcaaauuu

SEQ ID NO: 173 - RNA, 21 nt, RNAi-antisense strand
auuugcuugaauggcagauuu

SEQ ID NO: 174 - DNA, 21 bp, Homo sapiens
aatctgccattcaagcaaatg

SEQ ID NO: 175 - RNA, 21 nt, RNAi-sense strand
ucugccauucaagcaaauguu

SEQ ID NO: 176 - RNA, 21 nt, RNAi-antisense strand
cauuugcuugaauggcagauu

SEQ ID NO: 177 - DNA, 21 bp, Homo sapiens
aagaaatctcagttcaccttt

SEQ ID NO: 178 - RNA, 21 nt, RNAi-sense strand
gaaaucucaguucaccuuuuu

SEQ ID NO: 179 - RNA, 21 nt, RNAi-antisense strand
aaaggugaacugagauuucuu

SEQ ID NO: 180 - DNA, 21 bp, Homo sapiens
aaatctcagttcacctttcat

SEQ ID NO: 181 - RNA, 21 nt, RNAi-sense strand
aucucaguucaccuuucauuu

SEQ ID NO: 182 - RNA, 21 nt, RNAi-antisense strand
augaaaggugaacugagauuu

SEQ ID NO: 183 - DNA, 21 bp, Homo sapiens
aatctcagttcacctttcata

SEQ ID NO: 184 - RNA, 21 nt, RNAi-sense strand
ucucaguucaccuuucauauu

SEQ ID NO: 185 - RNA, 21 nt, RNAi-antisense strand
uaugaaaggugaacugagauu

SEQ ID NO: 186 - DNA, 21 bp, Homo sapiens
aagacaagtgaattctaacac

SEQ ID NO: 187 - RNA, 21 nt, RNAi-sense strand
gacaagugaauucuaacacuu

SEQ ID NO: 188 - RNA, 21 nt, RNAi-antisense strand
guguuagaauucacuugucuu

SEQ ID NO: 189 - DNA, 21 bp, Homo sapiens
aagtgaattctaacacttgca

SEQ ID NO: 190 - RNA, 21 nt, RNAi-sense strand
gugaauucuaacacuugcauu

SEQ ID NO: 191 - RNA, 21 nt, RNAi-antisense strand
ugcaaguguuagaauucacuu

SEQ ID NO: 192 - DNA, 21 bp, Homo sapiens
aattctaacacttgcaggagc

SEQ ID NO: 193 - RNA, 21 nt, RNAi-sense strand
uucuaacacuugcaggagcuu

SEQ ID NO: 194 - RNA, 21 nt, RNAi-antisense strand
gcuccugcaaguguuagaauu

SEQ ID NO: 195 - DNA, 21 bp, Homo sapiens
caagatagaacctttagatgt

SEQ ID NO: 196 - RNA, 21 nt, RNAi-sense strand
agauagaaccuuuagauguuu

SEQ ID NO: 197 - RNA, 21 nt, RNAi-antisense strand
acaucuaaagguucuaucuuu

SEQ ID NO: 198 - DNA, 21 bp, Homo sapiens
caccacgaaactagcaactgg

SEQ ID NO: 199 - RNA, 21 nt, RNAi-sense strand
ccacgaaacuagcaacugguu

SEQ ID NO: 200 - RNA, 21 nt, RNAi-antisense strand
ccaguugcuaguuucgugguu

SEQ ID NO: 201 - DNA, 21 bp, Homo sapiens
cacgaaactagcaactggaat

SEQ ID NO: 202 - RNA, 21 nt, RNAi-sense strand
cgaaacuagcaacuggaauuu

SEQ ID NO: 203 - RNA, 21 nt, RNAi-antisense strand
auuccaguugcuaguuucguu

SEQ ID NO: 204 - DNA, 21 bp, Homo sapiens
caactggaatgaagatagaaa

SEQ ID NO: 205 - RNA, 21 nt, RNAi-sense strand
acuggaaugaagauagaaauu

SEQ ID NO: 206 - RNA, 21 nt, RNAi-antisense strand
uuucuaucuucauuccaguuu

SEQ ID NO: 207 - DNA, 21 bp, Homo sapiens
caagtggttataactcagaca

SEQ ID NO: 208 - RNA, 21 nt, RNAi-sense strand
agugguuauaacucagacauu

SEQ ID NO: 209 - RNA, 21 nt, RNAi-antisense strand
ugucugaguuauaaccacuuu

SEQ ID NO: 210 - DNA, 21 bp, Homo sapiens
cagacaaactaatttgtcgag

SEQ ID NO: 211 - RNA, 21 nt, RNAi-sense strand
gacaaacuaauuugucgaguu

SEQ ID NO: 212 - RNA, 21 nt, RNAi-antisense strand
cucgacaaauuaguuugucuu

SEQ ID NO: 213 - DNA, 21 bp, Homo sapiens
  misc_feature (16)..(16): n is a, c, g, or t
caccggtttgcgacanaagtt

SEQ ID NO: 214 - RNA, 21 nt, RNAi-sense strand
  misc_feature (14)..(14): n is a, c, g, or u
ccgguuugcgacanaaguuuu

SEQ ID NO: 215 - RNA, 20 nt, RNAi-antisense strand
aacuuugucgcaaaccgguu

SEQ ID NO: 216 - DNA, 21 bp, Homo sapiens
  misc_feature (3)..(3): n is a, c, g, or t
canaagtttctcctgtcgcct

SEQ ID NO: 217 - RNA, 21 nt, RNAi-sense strand
  misc_feature (1)..(1): n is a, c, g, or u
naaguuucuccugucgccuuu

SEQ ID NO: 218 - RNA, 20 nt, RNAi-antisense strand
aggcgacaggagaaacuuuu

SEQ ID NO: 219 - DNA, 21 bp, Homo sapiens
cagacgtcagaatgattcctt

SEQ ID NO: 220 - RNA, 21 nt, RNAi-sense strand
gacgucagaaugauuccuuuu

SEQ ID NO: 221 - RNA, 21 nt, RNAi-antisense strand
aaggaaucauucugacgucuu

SEQ ID NO: 222 - DNA, 21 bp, Homo sapiens
cagaatgattccttgcagagc

SEQ ID NO: 223 - RNA, 21 nt, RNAi-sense strand
gaaugauuccuugcagagcuu

SEQ ID NO: 224 - RNA, 21 nt, RNAi-antisense strand
gcucugcaaggaaucauucuu

SEQ ID NO: 225 - DNA, 21 bp, Homo sapiens
cagtctagaagacaccaaatt

SEQ ID NO: 226 - RNA, 21 nt, RNAi-sense strand
gucuagaagacaccaaauuuu

SEQ ID NO: 227 - RNA, 21 nt, RNAi-antisense strand
aauuuggugucuucuagacuu

SEQ ID NO: 228 - DNA, 21 bp, Homo sapiens
caccaaattatgccgctgctt

SEQ ID NO: 229 - RNA, 21 nt, RNAi-sense strand
ccaaauuaugccgcugcuuuu

SEQ ID NO: 230 - RNA, 21 nt, RNAi-antisense strand
aagcagcggcauaauuugguu

SEQ ID NO: 231 - DNA, 21 bp, Homo sapiens
caaattatgccgctgcttatc

SEQ ID NO: 232 - RNA, 21 nt, RNAi-sense strand
aauuaugccgcugcuuaucuu

SEQ ID NO: 233 - RNA, 21 nt, RNAi-antisense strand
gauaagcagcggcauaauuuu

SEQ ID NO: 234 - DNA, 21 bp, Homo sapiens
cagcatcgtggtgtccttcct

SEQ ID NO: 235 - RNA, 21 nt, RNAi-sense strand
gcaucgugguguccuuccuuu

SEQ ID NO: 236 - RNA, 21 nt, RNAi-antisense strand
aggaaggacaccacgaugcuu

SEQ ID NO: 237 - DNA, 21 bp, Homo sapiens
catcgtggtgtccttcctcat

SEQ ID NO: 238 - RNA, 21 nt, RNAi-sense strand
ucgugguguccuuccucauuu

SEQ ID NO: 239 - RNA, 21 nt, RNAi-antisense strand
augaggaaggacaccacgauu

SEQ ID NO: 240 - DNA, 21 bp, Homo sapiens
cagtgatggctggcctctgct

SEQ ID NO: 241 - RNA, 21 nt, RNAi-sense strand
gugauggcuggccucugcuuu

SEQ ID NO: 242 - RNA, 21 nt, RNAi-antisense strand
agcagaggccagccaucacuu

SEQ ID NO: 243 - DNA, 21 bp, Homo sapiens
catatttgtacacctacgtga

SEQ ID NO: 244 - RNA, 21 nt, RNAi-sense strand
uauuuguacaccuacgugauu

SEQ ID NO: 245 - RNA, 21 nt, RNAi-antisense strand
ucacguagguguacaaauauu

SEQ ID NO: 246 - DNA, 21 bp, Homo sapiens
cacctacgtgactgtcggaga

SEQ ID NO: 247 - RNA, 21 nt, RNAi-sense strand
ccuacgugacugucggagauu

SEQ ID NO: 248 - RNA, 21 nt, RNAi-antisense strand
ucuccgacagucacguagguu

SEQ ID NO: 249 - DNA, 21 bp, Homo sapiens
catcactggctggaatctcat

SEQ ID NO: 250 - RNA, 21 nt, RNAi-sense strand
ucacuggcuggaaucucauuu

SEQ ID NO: 251 - RNA, 21 nt, RNAi-antisense strand
augagauuccagccagugauu

SEQ ID NO: 252 - DNA, 21 bp, Homo sapiens
catcaagtgttgcaagagcct

SEQ ID NO: 253 - RNA, 21 nt, RNAi-sense strand
ucaaguguugcaagagccuuu

SEQ ID NO: 254 - RNA, 21 nt, RNAi-antisense strand
aggcucuugcaacacuugauu

SEQ ID NO: 255 - DNA, 21 bp, Homo sapiens
caagtgttgcaagagcctgga

SEQ ID NO: 256 - RNA, 21 nt, RNAi-sense strand
aguguugcaagagccuggauu

SEQ ID NO: 257 - RNA, 21 nt, RNAi-antisense strand
uccaggcucuugcaacacuuu

SEQ ID NO: 258 - DNA, 21 bp, Homo sapiens
caagagcctggagtggcacct

SEQ ID NO: 259 - RNA, 21 nt, RNAi-sense strand
agagccuggaguggcaccuuu

SEQ ID NO: 260 - RNA, 21 nt, RNAi-antisense strand
aggugccacuccaggcucuuu

SEQ ID NO: 261 - DNA, 21 bp, Homo sapiens
cacctttgatgaacttcttag

SEQ ID NO: 262 - RNA, 21 nt, RNAi-sense strand
ccuuugaugaacuucuuaguu

SEQ ID NO: 263 - RNA, 21 nt, RNAi-antisense strand
cuaagaaguucaucaaagguu

SEQ ID NO: 264 - DNA, 21 bp, Homo sapiens
cagaatgaattacactggtct

SEQ ID NO: 265 - RNA, 21 nt, RNAi-sense strand
gaaugaauuacacuggucuuu

SEQ ID NO: 266 - RNA, 21 nt, RNAi-antisense strand
agaccaguguaauucauucuu

SEQ ID NO: 267 - DNA, 21 bp, Homo sapiens
cactggtcttgcagaatatcc

SEQ ID NO: 268 - RNA, 21 nt, RNAi-sense strand
cuggucuugcagaauauccuu

SEQ ID NO: 269 - RNA, 21 nt, RNAi-antisense strand
ggauauucugcaagaccaguu

SEQ ID NO: 270 - DNA, 21 bp, Homo sapiens
cacagctgttaatattctcgt

SEQ ID NO: 271 - RNA, 21 nt, RNAi-sense strand
cagcuguuaauauucucguuu

SEQ ID NO: 272 - RNA, 21 nt, RNAi-antisense strand
acgagaauauuaacagcuguu

SEQ ID NO: 273 - DNA, 21 bp, Homo sapiens
cagctgttaatattctcgtcc

SEQ ID NO: 274 - RNA, 21 nt, RNAi-sense strand
gcuguuaauauucucguccuu

SEQ ID NO: 275 - RNA, 21 nt, RNAi-antisense strand
ggacgagaauauuaacagcuu

SEQ ID NO: 276 - DNA, 21 bp, Homo sapiens
caaactggaagattagtgaag

SEQ ID NO: 277 - RNA, 21 nt, RNAi-sense strand
aacuggaagauuagugaaguu

SEQ ID NO: 278 - RNA, 21 nt, RNAi-antisense strand
cuucacuaaucuuccaguuuu

SEQ ID NO: 279 - DNA, 21 bp, Homo sapiens
cagcaagtgccagagagccac

SEQ ID NO: 280 - RNA, 21 nt, RNAi-sense strand
gcaagugccagagagccacuu

SEQ ID NO: 281 - RNA, 21 nt, RNAi-antisense strand
guggcucucuggcacuugcuu

SEQ ID NO: 282 - DNA, 21 bp, Homo sapiens
caagtgccagagagccacctt

SEQ ID NO: 283 - RNA, 21 nt, RNAi-sense strand
agugccagagagccaccuuuu

SEQ ID NO: 284 - RNA, 21 nt, RNAi-antisense strand
aagguggcucucuggcacuuu

SEQ ID NO: 285 - DNA, 21 bp, Homo sapiens
cattgcaacaactggtgaaga

SEQ ID NO: 286 - RNA, 21 nt, RNAi-sense strand
uugcaacaacuggugaagauu

SEQ ID NO: 287 - RNA, 21 nt, RNAi-antisense strand
ucuucaccaguuguugcaauu

SEQ ID NO: 288 - DNA, 21 bp, Homo sapiens
caacaactggtgaagaagttc

SEQ ID NO: 289 - RNA, 21 nt, RNAi-sense strand
acaacuggugaagaaguucuu

SEQ ID NO: 290 - RNA, 21 nt, RNAi-antisense strand
gaacuucuucaccaguuguuu

SEQ ID NO: 291 - DNA, 21 bp, Homo sapiens
caactggtgaagaagttcgga

SEQ ID NO: 292 - RNA, 21 nt, RNAi-sense strand
acuggugaagaaguucggauu

SEQ ID NO: 293 - RNA, 21 nt, RNAi-antisense strand
uccgaacuucuucaccaguuu

SEQ ID NO: 294 - DNA, 21 bp, Homo sapiens
cattggaattgtgacgtcttt

SEQ ID NO: 295 - RNA, 21 nt, RNAi-sense strand
uuggaauugugacgucuuuuu

SEQ ID NO: 296 - RNA, 21 nt, RNAi-antisense strand
aaagacgucacaauuccaauu

SEQ ID NO: 297 - DNA, 21 bp, Homo sapiens
cagctttaacacttatgatgc

SEQ ID NO: 298 - RNA, 21 nt, RNAi-sense strand
gcuuuaacacuuaugaugcuu

SEQ ID NO: 299 - RNA, 21 nt, RNAi-antisense strand
gcaucauaaguguuaaagcuu

SEQ ID NO: 300 - DNA, 21 bp, Homo sapiens
cacttatgatgccgtactacc

SEQ ID NO: 301 - RNA, 21 nt, RNAi-sense strand
cuuaugaugccguacuaccuu

SEQ ID NO: 302 - RNA, 21 nt, RNAi-antisense strand
gguaguacggcaucauaaguu

SEQ ID NO: 303 - DNA, 21 bp, Homo sapiens
caaatatgtcgtcgcagctgg

SEQ ID NO: 304 - RNA, 21 nt, RNAi-sense strand
aauaugucgucgcagcugguu

SEQ ID NO: 305 - RNA, 21 nt, RNAi-antisense strand
ccagcugcgacgacauauuuu

SEQ ID NO: 306 - DNA, 21 bp, Homo sapiens
cagctggttctctctgcgcct

SEQ ID NO: 307 - RNA, 21 nt, RNAi-sense strand
gcugguucucucugcgccuuu

SEQ ID NO: 308 - RNA, 21 nt, RNAi-antisense strand
aggcgcagagagaaccagcuu

SEQ ID NO: 309 - DNA, 21 bp, Homo sapiens
cagagtgagtaagaggcagtc

SEQ ID NO: 310 - RNA, 21 nt, RNAi-sense strand
gagugaguaagaggcagucuu

SEQ ID NO: 311 - RNA, 21 nt, RNAi-antisense strand
gacugccucuuacucacucuu

SEQ ID NO: 312 - DNA, 21 bp, Homo sapiens
cagtcaccagttgctgccacg

SEQ ID NO: 313 - RNA, 21 nt, RNAi-sense strand
gucaccaguugcugccacguu

SEQ ID NO: 314 - RNA, 21 nt, RNAi-antisense strand
cguggcagcaacuggugacuu

SEQ ID NO: 315 - DNA, 21 bp, Homo sapiens
caccagttgctgccacgttga

SEQ ID NO: 316 - RNA, 21 nt, RNAi-sense strand
ccaguugcugccacguugauu

SEQ ID NO: 317 - RNA, 21 nt, RNAi-antisense strand
ucaacguggcagcaacugguu

SEQ ID NO: 318 - DNA, 21 bp, Homo sapiens
cagttgctgccacgttgactg

SEQ ID NO: 319 - RNA, 21 nt, RNAi-sense strand
guugcugccacguugacuguu

SEQ ID NO: 320 - RNA, 21 nt, RNAi-antisense strand
cagucaacguggcagcaacuu

SEQ ID NO: 321 - DNA, 21 bp, Homo sapiens
catttctgctttgatggcctt

SEQ ID NO: 322 - RNA, 21 nt, RNAi-sense strand
uuucugcuuugauggccuuuu

SEQ ID NO: 323 - RNA, 21 nt, RNAi-antisense strand
aaggccaucaaagcagaaauu

SEQ ID NO: 324 - DNA, 21 bp, Homo sapiens
catgatgtccattggcacact

SEQ ID NO: 325 - RNA, 21 nt, RNAi-sense strand
ugauguccauuggcacacuuu

SEQ ID NO: 326 - RNA, 21 nt, RNAi-antisense strand
agugugccaauggacaucauu

SEQ ID NO: 327 - DNA, 21 bp, Homo sapiens
cattggcacactcatggccta

SEQ ID NO: 328 - RNA, 21 nt, RNAi-sense strand
uuggcacacucauggccuauu

SEQ ID NO: 329 - RNA, 21 nt, RNAi-antisense strand
uaggccaugagugugccaauu

SEQ ID NO: 330 - DNA, 21 bp, Homo sapiens
cacactcatggcctactctct

SEQ ID NO: 331 - RNA, 21 nt, RNAi-sense strand
cacucauggccuacucucuuu

SEQ ID NO: 332 - RNA, 21 nt, RNAi-antisense strand
agagaguaggccaugaguguu

SEQ ID NO: 333 - DNA, 21 bp, Homo sapiens
cactcatggcctactctctgg

SEQ ID NO: 334 - RNA, 21 nt, RNAi-sense strand
cucauggccuacucucugguu

SEQ ID NO: 335 - RNA, 21 nt, RNAi-antisense strand
ccagagaguaggccaugaguu

SEQ ID NO: 336 - DNA, 21 bp, Homo sapiens
catggcctactctctggtggc

SEQ ID NO: 337 - RNA, 21 nt, RNAi-sense strand
uggccuacucucugguggcuu

SEQ ID NO: 338 - RNA, 21 nt, RNAi-antisense strand
gccaccagagaguaggccauu

SEQ ID NO: 339 - DNA, 21 bp, Homo sapiens
cagcctgtgttctcatcctca

SEQ ID NO: 340 - RNA, 21 nt, RNAi-sense strand
gccuguguucucauccucauu

SEQ ID NO: 341 - RNA, 21 nt, RNAi-antisense strand
ugaggaugagaacacaggcuu

SEQ ID NO: 342 - DNA, 21 bp, Homo sapiens
catcctcaggtaccagcctgg

SEQ ID NO: 343 - RNA, 21 nt, RNAi-sense strand
uccucagguaccagccugguu

SEQ ID NO: 344 - RNA, 21 nt, RNAi-antisense strand
ccaggcugguaccugaggauu

SEQ ID NO: 345 - DNA, 21 bp, Homo sapiens
caggtaccagcctggcttatc

SEQ ID NO: 346 - RNA, 21 nt, RNAi-sense strand
gguaccagccuggcuuaucuu

SEQ ID NO: 347 - RNA, 21 nt, RNAi-antisense strand
gauaagccaggcugguaccuu

SEQ ID NO: 348 - DNA, 21 bp, Homo sapiens
cagcctggcttatcttacgac

SEQ ID NO: 349 - RNA, 21 nt, RNAi-sense strand
gccuggcuuaucuuacgacuu

SEQ ID NO: 350 - RNA, 21 nt, RNAi-antisense strand
gucguaagauaagccaggcuu

SEQ ID NO: 351 - DNA, 21 bp, Homo sapiens
caaatgttctcctgagaaaga

SEQ ID NO: 352 - RNA, 21 nt, RNAi-sense strand
aauguucuccugagaaagauu

SEQ ID NO: 353 - RNA, 21 nt, RNAi-antisense strand
ucuuucucaggagaacauuuu

SEQ ID NO: 354 - DNA, 21 bp, Homo sapiens
caggtcaccatgctgcagaga

SEQ ID NO: 355 - RNA, 21 nt, RNAi-sense strand
ggucaccaugcugcagagauu

SEQ ID NO: 356 - RNA, 21 nt, RNAi-antisense strand
ucucugcagcauggugaccuu

SEQ ID NO: 357 - DNA, 21 bp, Homo sapiens
caacacagcagtcagcttctc

SEQ ID NO: 358 - RNA, 21 nt, RNAi-sense strand
acacagcagucagcuucucuu

SEQ ID NO: 359 - RNA, 21 nt, RNAi-antisense strand
gagaagcugacugcuguguuu

SEQ ID NO: 360 - DNA, 21 bp, Homo sapiens
cacagcagtcagcttctctcg

SEQ ID NO: 361 - RNA, 21 nt, RNAi-sense strand
cagcagucagcuucucucguu

SEQ ID NO: 362 - RNA, 21 nt, RNAi-antisense strand
cgagagaagcugacugcuguu

SEQ ID NO: 363 - DNA, 21 bp, Homo sapiens
cagcagtcagcttctctcgtg

SEQ ID NO: 364 - RNA, 21 nt, RNAi-sense strand
gcagucagcuucucucguguu

SEQ ID NO: 365 - RNA, 21 nt, RNAi-antisense strand
cacgagagaagcugacugcuu

SEQ ID NO: 366 - DNA, 21 bp, Homo sapiens
cagtcagcttctctcgtgagc

SEQ ID NO: 367 - RNA, 21 nt, RNAi-sense strand
gucagcuucucucgugagcuu

SEQ ID NO: 368 - RNA, 21 nt, RNAi-antisense strand
gcucacgagagaagcugacuu

SEQ ID NO: 369 - DNA, 21 bp, Homo sapiens
cagcttctctcgtgagctttc

SEQ ID NO: 370 - RNA, 21 nt, RNAi-sense strand
gcuucucucgugagcuuucuu

SEQ ID NO: 371 - RNA, 21 nt, RNAi-antisense strand
gaaagcucacgagagaagcuu

SEQ ID NO: 372 - DNA, 21 bp, Homo sapiens
cacttacggagttcatgccat

SEQ ID NO: 373 - RNA, 21 nt, RNAi-sense strand
cuuacggaguucaugccauuu

SEQ ID NO: 374 - RNA, 21 nt, RNAi-antisense strand
auggcaugaacuccguaaguu

SEQ ID NO: 375 - DNA, 21 bp, Homo sapiens
catgccatcaccaggctggag

SEQ ID NO: 376 - RNA, 21 nt, RNAi-sense strand
ugccaucaccaggcuggaguu

SEQ ID NO: 377 - RNA, 21 nt, RNAi-antisense strand
cuccagccuggugauggcauu

SEQ ID NO: 378 - DNA, 21 bp, Homo sapiens
cacgctgtttcttgttctctt

SEQ ID NO: 379 - RNA, 21 nt, RNAi-sense strand
cgcuguuucuuguucucuuuu

SEQ ID NO: 380 - RNA, 21 nt, RNAi-antisense strand
aagagaacaagaaacagcguu

SEQ ID NO: 381 - DNA, 21 bp, Homo sapiens
catcgttctcaccatctggag

SEQ ID NO: 382 - RNA, 21 nt, RNAi-sense strand
ucguucucaccaucuggaguu

SEQ ID NO: 383 - RNA, 21 nt, RNAi-antisense strand
cuccagauggugagaacgauu

SEQ ID NO: 384 - DNA, 21 bp, Homo sapiens
catggttccattcttaccatt

SEQ ID NO: 385 - RNA, 21 nt, RNAi-sense strand
ugguuccauucuuaccauuuu

SEQ ID NO: 386 - RNA, 21 nt, RNAi-antisense strand
aaugguaagaauggaaccauu

SEQ ID NO: 387 - DNA, 21 bp, Homo sapiens
cagcgttcagcatcttggtga

SEQ ID NO: 388 - RNA, 21 nt, RNAi-sense strand
gcguucagcaucuuggugauu

SEQ ID NO: 389 - RNA, 21 nt, RNAi-antisense strand
ucaccaagaugcugaacgcuu

SEQ ID NO: 390 - DNA, 21 bp, Homo sapiens
cagcatcttggtgaacattta

SEQ ID NO: 391 - RNA, 21 nt, RNAi-sense strand
gcaucuuggugaacauuuauu

SEQ ID NO: 392 - RNA, 21 nt, RNAi-antisense strand
uaaauguucaccaagaugcuu

SEQ ID NO: 393 - DNA, 21 bp, Homo sapiens
catcttggtgaacatttactt

SEQ ID NO: 394 - RNA, 21 nt, RNAi-sense strand
ucuuggugaacauuuacuuuu

SEQ ID NO: 395 - RNA, 21 nt, RNAi-antisense strand
aaguaaauguucaccaagauu

SEQ ID NO: 396 - DNA, 21 bp, Homo sapiens
catttacttgatggtccagtt

SEQ ID NO: 397 - RNA, 21 nt, RNAi-sense strand
uuuacuugaugguccaguuuu

SEQ ID NO: 398 - RNA, 21 nt, RNAi-antisense strand
aacuggaccaucaaguaaauu

SEQ ID NO: 399 - DNA, 21 bp, Homo sapiens
cagttaagtgcagacacttgg

SEQ ID NO: 400 - RNA, 21 nt, RNAi-sense strand
guuaagugcagacacuugguu

SEQ ID NO: 401 - RNA, 21 nt, RNAi-antisense strand
ccaagugucugcacuuaacuu

SEQ ID NO: 402 - DNA, 21 bp, Homo sapiens
cagattcagcatttggatggc

SEQ ID NO: 403 - RNA, 21 nt, RNAi-sense strand
gauucagcauuuggauggcuu

SEQ ID NO: 404 - RNA, 21 nt, RNAi-antisense strand
gccauccaaaugcugaaucuu

SEQ ID NO: 405 - DNA, 21 bp, Homo sapiens
cagcatttggatggcaattgg

SEQ ID NO: 406 - RNA, 21 nt, RNAi-sense strand
gcauuuggauggcaauugguu

SEQ ID NO: 407 - RNA, 21 nt, RNAi-antisense strand
ccaauugccauccaaaugcuu

SEQ ID NO: 408 - DNA, 21 bp, Homo sapiens
catttggatggcaattggctt

SEQ ID NO: 409 - RNA, 21 nt, RNAi-sense strand
uuuggauggcaauuggcuuuu

SEQ ID NO: 410 - RNA, 21 nt, RNAi-antisense strand
aagccaauugccauccaaauu

SEQ ID NO: 411 - DNA, 21 bp, Homo sapiens
caattggcttcctgatttact

SEQ ID NO: 412 - RNA, 21 nt, RNAi-sense strand
auuggcuuccugauuuacuuu

SEQ ID NO: 413 - RNA, 21 nt, RNAi-antisense strand
aguaaaucaggaagccaauuu

SEQ ID NO: 414 - DNA, 21 bp, Homo sapiens
caatgaagaagatgcttatcc

SEQ ID NO: 415 - RNA, 21 nt, RNAi-sense strand
augaagaagaugcuuauccuu

SEQ ID NO: 416 - RNA, 21 nt, RNAi-antisense strand
ggauaagcaucuucuucauuu

SEQ ID NO: 417 - DNA, 21 bp, Homo sapiens
cagacaacgttcatgcagcag

SEQ ID NO: 418 - RNA, 21 nt, RNAi-sense strand
gacaacguucaugcagcaguu

SEQ ID NO: 419 - RNA, 21 nt, RNAi-antisense strand
cugcugcaugaacguugucuu

SEQ ID NO: 420 - DNA, 21 bp, Homo sapiens
caacgttcatgcagcagcaga

SEQ ID NO: 421 - RNA, 21 nt, RNAi-sense strand
acguucaugcagcagcagauu

SEQ ID NO: 422 - RNA, 21 nt, RNAi-antisense strand
ucugcugcugcaugaacguuu

SEQ ID NO: 423 - DNA, 21 bp, Homo sapiens
cattcaagcaaatgaccatca

SEQ ID NO: 424 - RNA, 21 nt, RNAi-sense strand
uucaagcaaaugaccaucauu

SEQ ID NO: 425 - RNA, 21 nt, RNAi-antisense strand
ugauggucauuugcuugaauu

SEQ ID NO: 426 - DNA, 21 bp, Homo sapiens
caagaaatctcagttcacctt

SEQ ID NO: 427 - RNA, 21 nt, RNAi-sense strand
agaaaucucaguucaccuuuu

SEQ ID NO: 428 - RNA, 21 nt, RNAi-antisense strand
aaggugaacugagauuucuuu

SEQ ID NO: 429 - DNA, 21 bp, Homo sapiens
cagttcacctttcatattcca

SEQ ID NO: 430 - RNA, 21 nt, RNAi-sense strand
guucaccuuucauauuccauu

SEQ ID NO: 431 - RNA, 21 nt, RNAi-antisense strand
uggaauaugaaaggugaacuu

SEQ ID NO: 432 - DNA, 21 bp, Homo sapiens
cacctttcatattccatgaaa

SEQ ID NO: 433 - RNA, 21 nt, RNAi-sense strand
ccuuucauauuccaugaaauu

SEQ ID NO: 434 - RNA, 21 nt, RNAi-antisense strand
uuucauggaauaugaaagguu

SEQ ID NO: 435 - DNA, 21 bp, Homo sapiens
caagtgaattctaacacttgc

SEQ ID NO: 436 - RNA, 21 nt, RNAi-sense strand
agugaauucuaacacuugcuu

SEQ ID NO: 437 - RNA, 21 nt, RNAi-antisense strand
gcaaguguuagaauucacuuu

SEQ ID NO: 438 - DNA, 21 bp, Homo sapiens
gaatcaagatagaacctttag

SEQ ID NO: 439 - RNA, 21 nt, RNAi-sense strand
aucaagauagaaccuuuaguu

SEQ ID NO: 440 - RNA, 21 nt, RNAi-antisense strand
cuaaagguucuaucuugauuu

SEQ ID NO: 441 - DNA, 21 bp, Homo sapiens
gatagaacctttagatgtctc

SEQ ID NO: 442 - RNA, 21 nt, RNAi-sense strand
uagaaccuuuagaugucucuu

SEQ ID NO: 443 - RNA, 21 nt, RNAi-antisense strand
gagacaucuaaagguucuauu

SEQ ID NO: 444 - DNA, 21 bp, Homo sapiens
gaacctttagatgtctcacca

SEQ ID NO: 445 - RNA, 21 nt, RNAi-sense strand
accuuuagaugucucaccauu

SEQ ID NO: 446 - RNA, 21 nt, RNAi-antisense strand
uggugagacaucuaaagguuu

SEQ ID NO: 447 - DNA, 21 bp, Homo sapiens
gatgtctcaccacgaaactag

SEQ ID NO: 448 - RNA, 21 nt, RNAi-sense strand
ugucucaccacgaaacuaguu

SEQ ID NO: 449 - RNA, 21 nt, RNAi-antisense strand
cuaguuucguggugagacauu

SEQ ID NO: 450 - DNA, 21 bp, Homo sapiens
gaaactagcaactggaatgaa

SEQ ID NO: 451 - RNA, 21 nt, RNAi-sense strand
aacuagcaacuggaaugaauu

SEQ ID NO: 452 - RNA, 21 nt, RNAi-antisense strand
uucauuccaguugcuaguuuu

SEQ ID NO: 453 - DNA, 21 bp, Homo sapiens
gaatgaagatagaaacaagtg

SEQ ID NO: 454 - RNA, 21 nt, RNAi-sense strand
augaagauagaaacaaguguu

SEQ ID NO: 455 - RNA, 21 nt, RNAi-antisense strand
cacuuguuucuaucuucauuu

SEQ ID NO: 456 - DNA, 21 bp, Homo sapiens
gaagatagaaacaagtggtta

SEQ ID NO: 457 - RNA, 21 nt, RNAi-sense strand
agauagaaacaagugguuauu

SEQ ID NO: 458 - RNA, 21 nt, RNAi-antisense strand
uaaccacuuguuucuaucuuu

SEQ ID NO: 459 - DNA, 21 bp, Homo sapiens
gaaacaagtggttataactca

SEQ ID NO: 460 - RNA, 21 nt, RNAi-sense strand
aacaagugguuauaacucauu

SEQ ID NO: 461 - RNA, 21 nt, RNAi-antisense strand
ugaguuauaaccacuuguuuu

SEQ ID NO: 462 - DNA, 21 bp, Homo sapiens
  misc_feature (5)..(5): n is a, c, g, or t
gacanaagtttctcctgtcgc

SEQ ID NO: 463 - RNA, 21 nt, RNAi-sense strand
  misc_feature (3)..(3): n is a, c, g, or u
canaaguuucuccugucgcuu

SEQ ID NO: 464 - RNA, 20 nt, RNAi-antisense strand
gcgacaggagaaacuuuguu

SEQ ID NO: 465 - DNA, 21 bp, Homo sapiens
gacgtcagaatgattccttgc

SEQ ID NO: 466 - RNA, 21 nt, RNAi-sense strand
cgucagaaugauuccuugcuu

SEQ ID NO: 467 - RNA, 21 nt, RNAi-antisense strand
gcaaggaaucauucugacguu

SEQ ID NO: 468 - DNA, 21 bp, Homo sapiens
gaatgattccttgcagagccg

SEQ ID NO: 469 - RNA, 21 nt, RNAi-sense strand
augauuccuugcagagccguu

SEQ ID NO: 470 - RNA, 21 nt, RNAi-antisense strand
cggcucugcaaggaaucauuu

SEQ ID NO: 471 - DNA, 21 bp, Homo sapiens
gacagtctagaagacaccaaa

SEQ ID NO: 472 - RNA, 21 nt, RNAi-sense strand
cagucuagaagacaccaaauu

SEQ ID NO: 473 - RNA, 21 nt, RNAi-antisense strand
uuuggugucuucuagacuguu

SEQ ID NO: 474 - DNA, 21 bp, Homo sapiens
gaagacaccaaattatgccgc

SEQ ID NO: 475 - RNA, 21 nt, RNAi-sense strand
agacaccaaauuaugccgcuu

SEQ ID NO: 476 - RNA, 21 nt, RNAi-antisense strand
gcggcauaauuuggugucuuu

SEQ ID NO: 477 - DNA, 21 bp, Homo sapiens
gacaccaaattatgccgctgc

SEQ ID NO: 478 - RNA, 21 nt, RNAi-sense strand
caccaaauuaugccgcugcuu

SEQ ID NO: 479 - RNA, 21 nt, RNAi-antisense strand
gcagcggcauaauuugguguu

SEQ ID NO: 480 - DNA, 21 bp, Homo sapiens
gatggctggcctctgctatgc

SEQ ID NO: 481 - RNA, 21 nt, RNAi-sense strand
uggcuggccucugcuaugcuu

SEQ ID NO: 482 - RNA, 21 nt, RNAi-antisense strand
gcauagcagaggccagccauu

SEQ ID NO: 483 - DNA, 21 bp, Homo sapiens
gataggtacatcaagtgttgc

SEQ ID NO: 484 - RNA, 21 nt, RNAi-sense strand
uagguacaucaaguguugcuu

SEQ ID NO: 485 - RNA, 21 nt, RNAi-antisense strand
gcaacacuugauguaccuauu

SEQ ID NO: 486 - DNA, 21 bp, Homo sapiens
gagcctggagtggcacctttg

SEQ ID NO: 487 - RNA, 21 nt, RNAi-sense strand
gccuggaguggcaccuuuguu

SEQ ID NO: 488 - RNA, 21 nt, RNAi-antisense strand
caaaggugccacuccaggcuu

SEQ ID NO: 489 - DNA, 21 bp, Homo sapiens
gagtggcacctttgatgaact

SEQ ID NO: 490 - RNA, 21 nt, RNAi-sense strand
guggcaccuuugaugaacuuu

SEQ ID NO: 491 - RNA, 21 nt, RNAi-antisense strand
aguucaucaaaggugccacuu

SEQ ID NO: 492 - DNA, 21 bp, Homo sapiens
gatgaacttcttagcaaacag

SEQ ID NO: 493 - RNA, 21 nt, RNAi-sense strand
ugaacuucuuagcaaacaguu

SEQ ID NO: 494 - RNA, 21 nt, RNAi-antisense strand
cuguuugcuaagaaguucauu

SEQ ID NO: 495 - DNA, 21 bp, Homo sapiens
gaacttcttagcaaacagatt

SEQ ID NO: 496 - RNA, 21 nt, RNAi-sense strand
acuucuuagcaaacagauuuu

SEQ ID NO: 497 - RNA, 21 nt, RNAi-antisense strand
aaucuguuugcuaagaaguuu

SEQ ID NO: 498 - DNA, 21 bp, Homo sapiens
gaggacatacttcagaatgaa

SEQ ID NO: 499 - RNA, 21 nt, RNAi-sense strand
ggacauacuucagaaugaauu

SEQ ID NO: 500 - RNA, 21 nt, RNAi-antisense strand
uucauucugaaguauguccuu

SEQ ID NO: 501 - DNA, 21 bp, Homo sapiens
gaatgaattacactggtcttg

SEQ ID NO: 502 - RNA, 21 nt, RNAi-sense strand
augaauuacacuggucuuguu

SEQ ID NO: 503 - RNA, 21 nt, RNAi-antisense strand
caagaccaguguaauucauuu

SEQ ID NO: 504 - DNA, 21 bp, Homo sapiens
gaattacactggtcttgcaga

SEQ ID NO: 505 - RNA, 21 nt, RNAi-sense strand
auuacacuggucuugcagauu

SEQ ID NO: 506 - RNA, 21 nt, RNAi-antisense strand
ucugcaagaccaguguaauuu

SEQ ID NO: 507 - DNA, 21 bp, Homo sapiens
gaataaagtcttcacagctgt

SEQ ID NO: 508 - RNA, 21 nt, RNAi-sense strand
auaaagucuucacagcuguuu

SEQ ID NO: 509 - RNA, 21 nt, RNAi-antisense strand
acagcugugaagacuuuauuu

SEQ ID NO: 510 - DNA, 21 bp, Homo sapiens
gaaaggaaatgtggcaaactg

SEQ ID NO: 511 - RNA, 21 nt, RNAi-sense strand
aaggaaauguggcaaacuguu

SEQ ID NO: 512 - RNA, 21 nt, RNAi-antisense strand
caguuugccacauuuccuuuu

SEQ ID NO: 513 - DNA, 21 bp, Homo sapiens
gaaatgtggcaaactggaaga

SEQ ID NO: 514 - RNA, 21 nt, RNAi-sense strand
aauguggcaaacuggaagauu

SEQ ID NO: 515 - RNA, 21 nt, RNAi-antisense strand
ucuuccaguuugccacauuuu

SEQ ID NO: 516 - DNA, 21 bp, Homo sapiens
gaagattagtgaagagtttct

SEQ ID NO: 517 - RNA, 21 nt, RNAi-sense strand
agauuagugaagaguuucuuu

SEQ ID NO: 518 - RNA, 21 nt, RNAi-antisense strand
agaaacucuucacuaaucuuu

SEQ ID NO: 519 - DNA, 21 bp, Homo sapiens
gattagtgaagagtttctcaa

SEQ ID NO: 520 - RNA, 21 nt, RNAi-sense strand
uuagugaagaguuucucaauu

SEQ ID NO: 521 - RNA, 21 nt, RNAi-antisense strand
uugagaaacucuucacuaauu

SEQ ID NO: 522 - DNA, 21 bp, Homo sapiens
gaacgttggctggtgctgcaa

SEQ ID NO: 523 - RNA, 21 nt, RNAi-sense strand
acguuggcuggugcugcaauu

SEQ ID NO: 524 - RNA, 21 nt, RNAi-antisense strand
uugcagcaccagccaacguuu

SEQ ID NO: 525 - DNA, 21 bp, Homo sapiens
gatttgactgcattgcaacaa

SEQ ID NO: 526 - RNA, 21 nt, RNAi-sense strand
uuugacugcauugcaacaauu

SEQ ID NO: 527 - RNA, 21 nt, RNAi-antisense strand
uuguugcaaugcagucaaauu

SEQ ID NO: 528 - DNA, 21 bp, Homo sapiens
gactgcattgcaacaactggt

SEQ ID NO: 529 - RNA, 21 nt, RNAi-sense strand
cugcauugcaacaacugguuu

SEQ ID NO: 530 - RNA, 21 nt, RNAi-antisense strand
accaguuguugcaaugcaguu

SEQ ID NO: 531 - DNA, 21 bp, Homo sapiens
gaattgtgacgtctttgcttg

SEQ ID NO: 532 - RNA, 21 nt, RNAi-sense strand
auugugacgucuuugcuuguu

SEQ ID NO: 533 - RNA, 21 nt, RNAi-antisense strand
caagcaaagacgucacaauuu

SEQ ID NO: 534 - DNA, 21 bp, Homo sapiens
gacgtctttgcttgtttgctt

SEQ ID NO: 535 - RNA, 21 nt, RNAi-sense strand
cgucuuugcuuguuugcuuuu

SEQ ID NO: 536 - RNA, 21 nt, RNAi-antisense strand
aagcaaacaagcaaagacguu

SEQ ID NO: 537 - DNA, 21 bp, Homo sapiens
gatgccgtactacctcctcga

SEQ ID NO: 538 - RNA, 21 nt, RNAi-sense strand
ugccguacuaccuccucgauu

SEQ ID NO: 539 - RNA, 21 nt, RNAi-antisense strand
ucgaggagguaguacggcauu

SEQ ID NO: 540 - DNA, 21 bp, Homo sapiens
gatggcttactgtttagattt

SEQ ID NO: 541 - RNA, 21 nt, RNAi-sense strand
uggcuuacuguuuagauuuuu

SEQ ID NO: 542 - RNA, 21 nt, RNAi-antisense strand
aaaucuaaacaguaagccauu

SEQ ID NO: 543 - DNA, 21 bp, Homo sapiens
gatttcttgccagagtgagta

SEQ ID NO: 544 - RNA, 21 nt, RNAi-sense strand
uuucuugccagagugaguauu

SEQ ID NO: 545 - RNA, 21 nt, RNAi-antisense strand
uacucacucuggcaagaaauu

SEQ ID NO: 546 - DNA, 21 bp, Homo sapiens
gagtgagtaagaggcagtcac

SEQ ID NO: 547 - RNA, 21 nt, RNAi-sense strand
gugaguaagaggcagucacuu

SEQ ID NO: 548 - RNA, 21 nt, RNAi-antisense strand
gugacugccucuuacucacuu

SEQ ID NO: 549 - DNA, 21 bp, Homo sapiens
gagtaagaggcagtcaccagt

SEQ ID NO: 550 - RNA, 21 nt, RNAi-sense strand
guaagaggcagucaccaguuu

SEQ ID NO: 551 - RNA, 21 nt, RNAi-antisense strand
acuggugacugccucuuacuu

SEQ ID NO: 552 - DNA, 21 bp, Homo sapiens
gaggcagtcaccagttgctgc

SEQ ID NO: 553 - RNA, 21 nt, RNAi-sense strand
ggcagucaccaguugcugcuu

SEQ ID NO: 554 - RNA, 21 nt, RNAi-antisense strand
gcagcaacuggugacugccuu

SEQ ID NO: 555 - DNA, 21 bp, Homo sapiens
gatggcctttctgtttgacct

SEQ ID NO: 556 - RNA, 21 nt, RNAi-sense strand
uggccuuucuguuugaccuuu

SEQ ID NO: 557 - RNA, 21 nt, RNAi-antisense strand
aggucaaacagaaaggccauu

SEQ ID NO: 558 - DNA, 21 bp, Homo sapiens
gacctgaaggcgcttgtggac

SEQ ID NO: 559 - RNA, 21 nt, RNAi-sense strand
ccugaaggcgcuuguggacuu

SEQ ID NO: 560 - RNA, 21 nt, RNAi-antisense strand
guccacaagcgccuucagguu

SEQ ID NO: 561 - DNA, 21 bp, Homo sapiens
gaaggcgcttgtggacatgat

SEQ ID NO: 562 - RNA, 21 nt, RNAi-sense strand
aggcgcuuguggacaugauuu

SEQ ID NO: 563 - RNA, 21 nt, RNAi-antisense strand
aucauguccacaagcgccuuu

SEQ ID NO: 564 - DNA, 21 bp, Homo sapiens
gacatgatgtccattggcaca

SEQ ID NO: 565 - RNA, 21 nt, RNAi-sense strand
caugauguccauuggcacauu

SEQ ID NO: 566 - RNA, 21 nt, RNAi-antisense strand
ugugccaauggacaucauguu

SEQ ID NO: 567 - DNA, 21 bp, Homo sapiens
gatgtccattggcacactcat

SEQ ID NO: 568 - RNA, 21 nt, RNAi-sense strand
uguccauuggcacacucauuu

SEQ ID NO: 569 - RNA, 21 nt, RNAi-antisense strand
augagugugccaauggacauu

SEQ ID NO: 570 - DNA, 21 bp, Homo sapiens
gagctttctggtaggattcct

SEQ ID NO: 571 - RNA, 21 nt, RNAi-sense strand
gcuuucugguaggauuccuuu

SEQ ID NO: 572 - RNA, 21 nt, RNAi-antisense strand
aggaauccuaccagaaagcuu

SEQ ID NO: 573 - DNA, 21 bp, Homo sapiens
gattcctagctttcctcgtgt

SEQ ID NO: 574 - RNA, 21 nt, RNAi-sense strand
uuccuagcuuuccucguguuu

SEQ ID NO: 575 - RNA, 21 nt, RNAi-antisense strand
acacgaggaaagcuaggaauu

SEQ ID NO: 576 - DNA, 21 bp, Homo sapiens
gagtgtcttgaccacttacgg

SEQ ID NO: 577 - RNA, 21 nt, RNAi-sense strand
gugucuugaccacuuacgguu

SEQ ID NO: 578 - RNA, 21 nt, RNAi-antisense strand
ccguaaguggucaagacacuu

SEQ ID NO: 579 - DNA, 21 bp, Homo sapiens
gaccacttacggagttcatgc

SEQ ID NO: 580 - RNA, 21 nt, RNAi-sense strand
ccacuuacggaguucaugcuu

SEQ ID NO: 581 - RNA, 21 nt, RNAi-antisense strand
gcaugaacuccguaagugguu

SEQ ID NO: 582 - DNA, 21 bp, Homo sapiens
gagttcatgccatcaccaggc

SEQ ID NO: 583 - RNA, 21 nt, RNAi-sense strand
guucaugccaucaccaggcuu

SEQ ID NO: 584 - RNA, 21 nt, RNAi-antisense strand
gccuggugauggcaugaacuu

SEQ ID NO: 585 - DNA, 21 bp, Homo sapiens
gaacatttacttgatggtcca

SEQ ID NO: 586 - RNA, 21 nt, RNAi-sense strand
acauuuacuugaugguccauu

SEQ ID NO: 587 - RNA, 21 nt, RNAi-antisense strand
uggaccaucaaguaaauguuu

SEQ ID NO: 588 - DNA, 21 bp, Homo sapiens
gatggtccagttaagtgcaga

SEQ ID NO: 589 - RNA, 21 nt, RNAi-sense strand
ugguccaguuaagugcagauu

SEQ ID NO: 590 - RNA, 21 nt, RNAi-antisense strand
ucugcacuuaacuggaccauu

SEQ ID NO: 591 - DNA, 21 bp, Homo sapiens
gattcagcatttggatggcaa

SEQ ID NO: 592 - RNA, 21 nt, RNAi-sense strand
uucagcauuuggauggcaauu

SEQ ID NO: 593 - RNA, 21 nt, RNAi-antisense strand
uugccauccaaaugcugaauu

SEQ ID NO: 594 - DNA, 21 bp, Homo sapiens
gatggcaattggcttcctgat

SEQ ID NO: 595 - RNA, 21 nt, RNAi-sense strand
uggcaauuggcuuccugauuu

SEQ ID NO: 596 - RNA, 21 nt, RNAi-antisense strand
aucaggaagccaauugccauu

SEQ ID NO: 597 - DNA, 21 bp, Homo sapiens
gaagaagatgcttatccagac

SEQ ID NO: 598 - RNA, 21 nt, RNAi-sense strand
agaagaugcuuauccagacuu

SEQ ID NO: 599 - RNA, 21 nt, RNAi-antisense strand
gucuggauaagcaucuucuuu

SEQ ID NO: 600 - DNA, 21 bp, Homo sapiens
gaagatgcttatccagacaac

SEQ ID NO: 601 - RNA, 21 nt, RNAi-sense strand
agaugcuuauccagacaacuu

SEQ ID NO: 602 - RNA, 21 nt, RNAi-antisense strand
guugucuggauaagcaucuuu

SEQ ID NO: 603 - DNA, 21 bp, Homo sapiens
gatgcttatccagacaacgtt

SEQ ID NO: 604 - RNA, 21 nt, RNAi-sense strand
ugcuuauccagacaacguuuu

SEQ ID NO: 605 - RNA, 21 nt, RNAi-antisense strand
aacguugucuggauaagcauu

SEQ ID NO: 606 - DNA, 21 bp, Homo sapiens
gacaacgttcatgcagcagca

SEQ ID NO: 607 - RNA, 21 nt, RNAi-sense strand
caacguucaugcagcagcauu

SEQ ID NO: 608 - RNA, 21 nt, RNAi-antisense strand
ugcugcugcaugaacguuguu

SEQ ID NO: 609 - DNA, 21 bp, Homo sapiens
gaaatctcagttcacctttca

SEQ ID NO: 610 - RNA, 21 nt, RNAi-sense strand
aaucucaguucaccuuucauu

SEQ ID NO: 611 - RNA, 21 nt, RNAi-antisense strand
ugaaaggugaacugagauuuu

SEQ ID NO: 612 - DNA, 21 bp, Homo sapiens
gacaagtgaattctaacactt

SEQ ID NO: 613 - RNA, 21 nt, RNAi-sense strand
caagugaauucuaacacuuuu

SEQ ID NO: 614 - RNA, 21 nt, RNAi-antisense strand
aaguguuagaauucacuuguu

SEQ ID NO: 615 - DNA, 21 bp, Homo sapiens
gaattctaacacttgcaggag

SEQ ID NO: 616 - RNA, 21 nt, RNAi-sense strand
auucuaacacuugcaggaguu

SEQ ID NO: 617 - RNA, 21 nt, RNAi-antisense strand
cuccugcaaguguuagaauuu

SEQ ID NO: 618 - DNA, 21 bp, Homo sapiens
tagaacctttagatgtctcac

SEQ ID NO: 619 - RNA, 21 nt, RNAi-sense strand
gaaccuuuagaugucucacuu

SEQ ID NO: 620 - RNA, 21 nt, RNAi-antisense strand
gugagacaucuaaagguucuu

SEQ ID NO: 621 - DNA, 21 bp, Homo sapiens
tagatgtctcaccacgaaact

SEQ ID NO: 622 - RNA, 21 nt, RNAi-sense strand
gaugucucaccacgaaacuuu

SEQ ID NO: 623 - RNA, 21 nt, RNAi-antisense strand
aguuucguggugagacaucuu

SEQ ID NO: 624 - DNA, 21 bp, Homo sapiens
tagcaactggaatgaagatag

SEQ ID NO: 625 - RNA, 21 nt, RNAi-sense strand
gcaacuggaaugaagauaguu

SEQ ID NO: 626 - RNA, 21 nt, RNAi-antisense strand
cuaucuucauuccaguugcuu

SEQ ID NO: 627 - DNA, 21 bp, Homo sapiens
tagaagacaccaaattatgcc

SEQ ID NO: 628 - RNA, 21 nt, RNAi-sense strand
gaagacaccaaauuaugccuu

SEQ ID NO: 629 - RNA, 21 nt, RNAi-antisense strand
ggcauaauuuggugucuucuu

SEQ ID NO: 630 - DNA, 21 bp, Homo sapiens
tatgccgctgcttatccacca

SEQ ID NO: 631 - RNA, 21 nt, RNAi-sense strand
ugccgcugcuuauccaccauu

SEQ ID NO: 632 - RNA, 21 nt, RNAi-antisense strand
ugguggauaagcagcggcauu

SEQ ID NO: 633 - DNA, 21 bp, Homo sapiens
tatccaccatggacctcattg

SEQ ID NO: 634 - RNA, 21 nt, RNAi-sense strand
uccaccauggaccucauuguu

SEQ ID NO: 635 - RNA, 21 nt, RNAi-antisense strand
caaugagguccaugguggauu

SEQ ID NO: 636 - DNA, 21 bp, Homo sapiens
tatttgtacacctacgtgact

SEQ ID NO: 637 - RNA, 21 nt, RNAi-sense strand
uuuguacaccuacgugacuuu

SEQ ID NO: 638 - RNA, 21 nt, RNAi-antisense strand
agucacguagguguacaaauu

SEQ ID NO: 639 - DNA, 21 bp, Homo sapiens
tacacctacgtgactgtcgga

SEQ ID NO: 640 - RNA, 21 nt, RNAi-sense strand
caccuacgugacugucggauu

SEQ ID NO: 641 - RNA, 21 nt, RNAi-antisense strand
uccgacagucacguagguguu

SEQ ID NO: 642 - DNA, 21 bp, Homo sapiens
tacgtgactgtcggagagctg

SEQ ID NO: 643 - RNA, 21 nt, RNAi-sense strand
cgugacugucggagagcuguu

SEQ ID NO: 644 - RNA, 21 nt, RNAi-antisense strand
cagcucuccgacagucacguu

SEQ ID NO: 645 - DNA, 21 bp, Homo sapiens
tatcgtatgtgataggtacat

SEQ ID NO: 646 - RNA, 21 nt, RNAi-sense strand
ucguaugugauagguacauuu

SEQ ID NO: 647 - RNA, 21 nt, RNAi-antisense strand
auguaccuaucacauacgauu

SEQ ID NO: 648 - DNA, 21 bp, Homo sapiens
tatgtgataggtacatcaagt

SEQ ID NO: 649 - RNA, 21 nt, RNAi-sense strand
ugugauagguacaucaaguuu

SEQ ID NO: 650 - RNA, 21 nt, RNAi-antisense strand
acuugauguaccuaucacauu

SEQ ID NO: 651 - DNA, 21 bp, Homo sapiens
taggtacatcaagtgttgcaa

SEQ ID NO: 652 - RNA, 21 nt, RNAi-sense strand
gguacaucaaguguugcaauu

SEQ ID NO: 653 - RNA, 21 nt, RNAi-antisense strand
uugcaacacuugauguaccuu

SEQ ID NO: 654 - DNA, 21 bp, Homo sapiens
tacatcaagtgttgcaagagc

SEQ ID NO: 655 - RNA, 21 nt, RNAi-sense strand
caucaaguguugcaagagcuu

SEQ ID NO: 656 - RNA, 21 nt, RNAi-antisense strand
gcucuugcaacacuugauguu

SEQ ID NO: 657 - DNA, 21 bp, Homo sapiens
tagcaaacagattggtcagtt

SEQ ID NO: 658 - RNA, 21 nt, RNAi-sense strand
gcaaacagauuggucaguuuu

SEQ ID NO: 659 - RNA, 21 nt, RNAi-antisense strand
aacugaccaaucuguuugcuu

SEQ ID NO: 660 - DNA, 21 bp, Homo sapiens
tacactggtcttgcagaatat

SEQ ID NO: 661 - RNA, 21 nt, RNAi-sense strand
cacuggucuugcagaauauuu

SEQ ID NO: 662 - RNA, 21 nt, RNAi-antisense strand
auauucugcaagaccaguguu

SEQ ID NO: 663 - DNA, 21 bp, Homo sapiens
tatattacttctagcaggtct

SEQ ID NO: 664 - RNA, 21 nt, RNAi-sense strand
uauuacuucuagcaggucuuu

SEQ ID NO: 665 - RNA, 21 nt, RNAi-antisense strand
agaccugcuagaaguaauauu

SEQ ID NO: 666 - DNA, 21 bp, Homo sapiens
tattacttctagcaggtcttt

SEQ ID NO: 667 - RNA, 21 nt, RNAi-sense strand
uuacuucuagcaggucuuuuu

SEQ ID NO: 668 - RNA, 21 nt, RNAi-antisense strand
aaagaccugcuagaaguaauu

SEQ ID NO: 669 - DNA, 21 bp, Homo sapiens
taaagtcttcacagctgttaa

SEQ ID NO: 670 - RNA, 21 nt, RNAi-sense strand
aagucuucacagcuguuaauu

SEQ ID NO: 671 - RNA, 21 nt, RNAi-antisense strand
uuaacagcugugaagacuuuu

SEQ ID NO: 672 - DNA, 21 bp, Homo sapiens
taatattctcgtccttctgtt

SEQ ID NO: 673 - RNA, 21 nt, RNAi-sense strand
auauucucguccuucuguuuu

SEQ ID NO: 674 - RNA, 21 nt, RNAi-antisense strand
aacagaaggacgagaauauuu

SEQ ID NO: 675 - DNA, 21 bp, Homo sapiens
tattctcgtccttctgtttgt

SEQ ID NO: 676 - RNA, 21 nt, RNAi-sense strand
uucucguccuucuguuuguuu

SEQ ID NO: 677 - RNA, 21 nt, RNAi-antisense strand
acaaacagaaggacgagaauu

SEQ ID NO: 678 - DNA, 21 bp, Homo sapiens
tatatcagcaagtgccagaga

SEQ ID NO: 679 - RNA, 21 nt, RNAi-sense strand
uaucagcaagugccagagauu

SEQ ID NO: 680 - RNA, 21 nt, RNAi-antisense strand
ucucuggcacuugcugauauu

SEQ ID NO: 681 - DNA, 21 bp, Homo sapiens
tatcagcaagtgccagagagc

SEQ ID NO: 682 - RNA, 21 nt, RNAi-sense strand
ucagcaagugccagagagcuu

SEQ ID NO: 683 - RNA, 21 nt, RNAi-antisense strand
gcucucuggcacuugcugauu

SEQ ID NO: 684 - DNA, 21 bp, Homo sapiens
taacacttatgatgccgtact

SEQ ID NO: 685 - RNA, 21 nt, RNAi-sense strand
acacuuaugaugccguacuuu

SEQ ID NO: 686 - RNA, 21 nt, RNAi-antisense strand
aguacggcaucauaaguguuu

SEQ ID NO: 687 - DNA, 21 bp, Homo sapiens
tatgatgccgtactacctcct

SEQ ID NO: 688 - RNA, 21 nt, RNAi-sense strand
ugaugccguacuaccuccuuu

SEQ ID NO: 689 - RNA, 21 nt, RNAi-antisense strand
aggagguaguacggcaucauu

SEQ ID NO: 690 - DNA, 21 bp, Homo sapiens
tatgtcgtcgcagctggttct

SEQ ID NO: 691 - RNA, 21 nt, RNAi-sense strand
ugucgucgcagcugguucuuu

SEQ ID NO: 692 - RNA, 21 nt, RNAi-antisense strand
agaaccagcugcgacgacauu

SEQ ID NO: 693 - DNA, 21 bp, Homo sapiens
tactgtttagatttcttgcca

SEQ ID NO: 694 - RNA, 21 nt, RNAi-sense strand
cuguuuagauuucuugccauu

SEQ ID NO: 695 - RNA, 21 nt, RNAi-antisense strand
uggcaagaaaucuaaacaguu

SEQ ID NO: 696 - DNA, 21 bp, Homo sapiens
tagatttcttgccagagtgag

SEQ ID NO: 697 - RNA, 21 nt, RNAi-sense strand
gauuucuugccagagugaguu

SEQ ID NO: 698 - RNA, 21 nt, RNAi-antisense strand
cucacucuggcaagaaaucuu

SEQ ID NO: 699 - DNA, 21 bp, Homo sapiens
taagaggcagtcaccagttgc

SEQ ID NO: 700 - RNA, 21 nt, RNAi-sense strand
agaggcagucaccaguugcuu

SEQ ID NO: 701 - RNA, 21 nt, RNAi-antisense strand
gcaacuggugacugccucuuu

SEQ ID NO: 702 - DNA, 21 bp, Homo sapiens
tactctctggtggcagcctgt

SEQ ID NO: 703 - RNA, 21 nt, RNAi-sense strand
cucucugguggcagccuguuu

SEQ ID NO: 704 - RNA, 21 nt, RNAi-antisense strand
acaggcugccaccagagaguu

SEQ ID NO: 705 - DNA, 21 bp, Homo sapiens
taccagcctggcttatcttac

SEQ ID NO: 706 - RNA, 21 nt, RNAi-sense strand
ccagccuggcuuaucuuacuu

SEQ ID NO: 707 - RNA, 21 nt, RNAi-antisense strand
guaagauaagccaggcugguu

SEQ ID NO: 708 - DNA, 21 bp, Homo sapiens
taggattcctagctttcctcg

SEQ ID NO: 709 - RNA, 21 nt, RNAi-sense strand
ggauuccuagcuuuccucguu

SEQ ID NO: 710 - RNA, 21 nt, RNAi-antisense strand
cgaggaaagcuaggaauccuu

SEQ ID NO: 711 - DNA, 21 bp, Homo sapiens
tacggagttcatgccatcacc

SEQ ID NO: 712 - RNA, 21 nt, RNAi-sense strand
cggaguucaugccaucaccuu

SEQ ID NO: 713 - RNA, 21 nt, RNAi-antisense strand
ggugauggcaugaacuccguu

SEQ ID NO: 714 - DNA, 21 bp, Homo sapiens
tagccttcatggttccattct

SEQ ID NO: 715 - RNA, 21 nt, RNAi-sense strand
gccuucaugguuccauucuuu

SEQ ID NO: 716 - RNA, 21 nt, RNAi-antisense strand
agaauggaaccaugaaggcuu

SEQ ID NO: 717 - DNA, 21 bp, Homo sapiens
tacttgatggtccagttaagt

SEQ ID NO: 718 - RNA, 21 nt, RNAi-sense strand
cuugaugguccaguuaaguuu

SEQ ID NO: 719 - RNA, 21 nt, RNAi-antisense strand
acuuaacuggaccaucaaguu

SEQ ID NO: 720 - DNA, 21 bp, Homo sapiens
tatggcattagacacagcctg

SEQ ID NO: 721 - RNA, 21 nt, RNAi-sense strand
uggcauuagacacagccuguu

SEQ ID NO: 722 - RNA, 21 nt, RNAi-antisense strand
caggcugugucuaaugccauu

SEQ ID NO: 723 - DNA, 21 bp, Homo sapiens
tatccagacaacgttcatgca

SEQ ID NO: 724 - RNA, 21 nt, RNAi-sense strand
uccagacaacguucaugcauu

SEQ ID NO: 725 - RNA, 21 nt, RNAi-antisense strand
ugcaugaacguugucuggauu

SEQ ID NO: 726 - DNA, 21 bp, Homo sapiens
aagtgcagcaaagagaagtgt

SEQ ID NO: 727 - RNA, 21 nt, RNAi-sense strand
gugcagcaaagagaaguguuu

SEQ ID NO: 728 - RNA, 21 nt, RNAi-antisense strand
acacuucucuuugcugcacuu

SEQ ID NO: 729 - DNA, 21 bp, Homo sapiens
aaagagaagtgtcagagcatg

SEQ ID NO: 730 - RNA, 21 nt, RNAi-sense strand
agagaagugucagagcauguu

SEQ ID NO: 731 - RNA, 21 nt, RNAi-antisense strand
caugcucugacacuucucuuu

SEQ ID NO: 732 - DNA, 21 bp, Homo sapiens
aagagaagtgtcagagcatga

SEQ ID NO: 733 - RNA, 21 nt, RNAi-sense strand
gagaagugucagagcaugauu

SEQ ID NO: 734 - RNA, 21 nt, RNAi-antisense strand
ucaugcucugacacuucucuu

SEQ ID NO: 735 - DNA, 21 bp, Homo sapiens
aagtgtcagagcatgagcgcc

SEQ ID NO: 736 - RNA, 21 nt, RNAi-sense strand
gugucagagcaugagcgccuu

SEQ ID NO: 737 - RNA, 21 nt, RNAi-antisense strand
ggcgcucaugcucugacacuu

SEQ ID NO: 738 - DNA, 21 bp, Homo sapiens
aaaggctggtctgcttgagaa

SEQ ID NO: 739 - RNA, 21 nt, RNAi-sense strand
aggcuggucugcuugagaauu

SEQ ID NO: 740 - RNA, 21 nt, RNAi-antisense strand
uucucaagcagaccagccuuu

SEQ ID NO: 741 - DNA, 21 bp, Homo sapiens
aaggctggtctgcttgagaaa

SEQ ID NO: 742 - RNA, 21 nt, RNAi-sense strand
ggcuggucugcuugagaaauu

SEQ ID NO: 743 - RNA, 21 nt, RNAi-antisense strand
uuucucaagcagaccagccuu

SEQ ID NO: 744 - DNA, 21 bp, Homo sapiens
aacttaaagaacaagagtgtg

SEQ ID NO: 745 - RNA, 21 nt, RNAi-sense strand
cuuaaagaacaagaguguguu

SEQ ID NO: 746 - RNA, 21 nt, RNAi-antisense strand
cacacucuuguucuuuaaguu

SEQ ID NO: 747 - DNA, 21 bp, Homo sapiens
aaagaacaagagtgtgatgtg

SEQ ID NO: 748 - RNA, 21 nt, RNAi-sense strand
agaacaagagugugauguguu

SEQ ID NO: 749 - RNA, 21 nt, RNAi-antisense strand
cacaucacacucuuguucuuu

SEQ ID NO: 750 - DNA, 21 bp, Homo sapiens
aagaacaagagtgtgatgtga

SEQ ID NO: 751 - RNA, 21 nt, RNAi-sense strand
gaacaagagugugaugugauu

SEQ ID NO: 752 - RNA, 21 nt, RNAi-antisense strand
ucacaucacacucuuguucuu

SEQ ID NO: 753 - DNA, 21 bp, Homo sapiens
aacaagagtgtgatgtgaagg

SEQ ID NO: 754 - RNA, 21 nt, RNAi-sense strand
caagagugugaugugaagguu

SEQ ID NO: 755 - RNA, 21 nt, RNAi-antisense strand
ccuucacaucacacucuuguu

SEQ ID NO: 756 - DNA, 21 bp, Homo sapiens
aagagtgtgatgtgaaggatt

SEQ ID NO: 757 - RNA, 21 nt, RNAi-sense strand
gagugugaugugaaggauuuu

SEQ ID NO: 758 - RNA, 21 nt, RNAi-antisense strand
aauccuucacaucacacucuu

SEQ ID NO: 759 - DNA, 21 bp, Homo sapiens
aaattgtgaagaatccaaggt

SEQ ID NO: 760 - RNA, 21 nt, RNAi-sense strand
auugugaagaauccaagguuu

SEQ ID NO: 761 - RNA, 21 nt, RNAi-antisense strand
accuuggauucuucacaauuu

SEQ ID NO: 762 - DNA, 21 bp, Homo sapiens
aattgtgaagaatccaaggtc

SEQ ID NO: 763 - RNA, 21 nt, RNAi-sense strand
uugugaagaauccaaggucuu

SEQ ID NO: 764 - RNA, 21 nt, RNAi-antisense strand
gaccuuggauucuucacaauu

SEQ ID NO: 765 - DNA, 21 bp, Homo sapiens
aaagcaagcgagcagctggct

SEQ ID NO: 766 - RNA, 21 nt, RNAi-sense strand
agcaagcgagcagcuggcuuu

SEQ ID NO: 767 - RNA, 21 nt, RNAi-antisense strand
agccagcugcucgcuugcuuu

SEQ ID NO: 768 - DNA, 21 bp, Homo sapiens
aagcaagcgagcagctggctg

SEQ ID NO: 769 - RNA, 21 nt, RNAi-sense strand
gcaagcgagcagcuggcuguu

SEQ ID NO: 770 - RNA, 21 nt, RNAi-antisense strand
cagccagcugcucgcuugcuu

SEQ ID NO: 771 - DNA, 21 bp, Homo sapiens
aagcgagcagctggctggcaa

SEQ ID NO: 772 - RNA, 21 nt, RNAi-sense strand
gcgagcagcuggcuggcaauu

SEQ ID NO: 773 - RNA, 21 nt, RNAi-antisense strand
uugccagccagcugcucgcuu

SEQ ID NO: 774 - DNA, 21 bp, Homo sapiens
aaggtggcagaagtcaagaag

SEQ ID NO: 775 - RNA, 21 nt, RNAi-sense strand
gguggcagaagucaagaaguu

SEQ ID NO: 776 - RNA, 21 nt, RNAi-antisense strand
cuucuugacuucugccaccuu

SEQ ID NO: 777 - DNA, 21 bp, Homo sapiens
aagtcaagaagaacggaagaa

SEQ ID NO: 778 - RNA, 21 nt, RNAi-sense strand
gucaagaagaacggaagaauu

SEQ ID NO: 779 - RNA, 21 nt, RNAi-antisense strand
uucuuccguucuucuugacuu

SEQ ID NO: 780 - DNA, 21 bp, Homo sapiens
aagaagaacggaagaatcagc

SEQ ID NO: 781 - RNA, 21 nt, RNAi-sense strand
gaagaacggaagaaucagcuu

SEQ ID NO: 782 - RNA, 21 nt, RNAi-antisense strand
gcugauucuuccguucuucuu

SEQ ID NO: 783 - DNA, 21 bp, Homo sapiens
aagaacggaagaatcagcctg

SEQ ID NO: 784 - RNA, 21 nt, RNAi-sense strand
gaacggaagaaucagccuguu

SEQ ID NO: 785 - RNA, 21 nt, RNAi-antisense strand
caggcugauucuuccguucuu

SEQ ID NO: 786 - DNA, 21 bp, Homo sapiens
aacggaagaatcagcctggtg

SEQ ID NO: 787 - RNA, 21 nt, RNAi-sense strand
cggaagaaucagccugguguu

SEQ ID NO: 788 - RNA, 21 nt, RNAi-antisense strand
caccaggcugauucuuccguu

SEQ ID NO: 789 - DNA, 21 bp, Homo sapiens
aattggaagcatctctggcca

SEQ ID NO: 790 - RNA, 21 nt, RNAi-sense strand
uuggaagcaucucuggccauu

SEQ ID NO: 791 - RNA, 21 nt, RNAi-antisense strand
uggccagagaugcuuccaauu

SEQ ID NO: 792 - DNA, 21 bp, Homo sapiens
aagcatctctggccatgccag

SEQ ID NO: 793 - RNA, 21 nt, RNAi-sense strand
gcaucucuggccaugccaguu

SEQ ID NO: 794 - RNA, 21 nt, RNAi-antisense strand
cuggcauggccagagaugcuu

SEQ ID NO: 795 - DNA, 21 bp, Homo sapiens
aacactccactgacaaccaca

SEQ ID NO: 796 - RNA, 21 nt, RNAi-sense strand
cacuccacugacaaccacauu

SEQ ID NO: 797 - RNA, 21 nt, RNAi-antisense strand
ugugguugucaguggaguguu

SEQ ID NO: 798 - DNA, 21 bp, Homo sapiens
aaccacaagtggaaacttgca

SEQ ID NO: 799 - RNA, 21 nt, RNAi-sense strand
ccacaaguggaaacuugcauu

SEQ ID NO: 800 - RNA, 21 nt, RNAi-antisense strand
ugcaaguuuccacuugugguu

SEQ ID NO: 801 - DNA, 21 bp, Homo sapiens
aagtggaaacttgcatggaca

SEQ ID NO: 802 - RNA, 21 nt, RNAi-sense strand
guggaaacuugcauggacauu

SEQ ID NO: 803 - RNA, 21 nt, RNAi-antisense strand
uguccaugcaaguuuccacuu

SEQ ID NO: 804 - DNA, 21 bp, Homo sapiens
aaacttgcatggacaacctgt

SEQ ID NO: 805 - RNA, 21 nt, RNAi-sense strand
acuugcauggacaaccuguuu

SEQ ID NO: 806 - RNA, 21 nt, RNAi-antisense strand
acagguuguccaugcaaguuu

SEQ ID NO: 807 - DNA, 21 bp, Homo sapiens
aacttgcatggacaacctgta

SEQ ID NO: 808 - RNA, 21 nt, RNAi-sense strand
cuugcauggacaaccuguauu

SEQ ID NO: 809 - RNA, 21 nt, RNAi-antisense strand
uacagguuguccaugcaaguu

SEQ ID NO: 810 - DNA, 21 bp, Homo sapiens
aacctgtatctttcctcctga

SEQ ID NO: 811 - RNA, 21 nt, RNAi-sense strand
ccuguaucuuuccuccugauu

SEQ ID NO: 812 - RNA, 21 nt, RNAi-antisense strand
ucaggaggaaagauacagguu

SEQ ID NO: 813 - DNA, 21 bp, Homo sapiens
aaggatattgtgtatattggc

SEQ ID NO: 814 - RNA, 21 nt, RNAi-sense strand
ggauauuguguauauuggcuu

SEQ ID NO: 815 - RNA, 21 nt, RNAi-antisense strand
gccaauauacacaauauccuu

SEQ ID NO: 816 - DNA, 21 bp, Homo sapiens
aatgactgaagtggacagact

SEQ ID NO: 817 - RNA, 21 nt, RNAi-sense strand
ugacugaaguggacagacuuu

SEQ ID NO: 818 - RNA, 21 nt, RNAi-antisense strand
agucuguccacuucagucauu

SEQ ID NO: 819 - DNA, 21 bp, Homo sapiens
aagtggacagactaggaattg

SEQ ID NO: 820 - RNA, 21 nt, RNAi-sense strand
guggacagacuaggaauuguu

SEQ ID NO: 821 - RNA, 21 nt, RNAi-antisense strand
caauuccuagucuguccacuu

SEQ ID NO: 822 - DNA, 21 bp, Homo sapiens
aattggcaaggtgatggaaga

SEQ ID NO: 823 - RNA, 21 nt, RNAi-sense strand
uuggcaaggugauggaagauu

SEQ ID NO: 824 - RNA, 21 nt, RNAi-antisense strand
ucuuccaucaccuugccaauu

SEQ ID NO: 825 - DNA, 21 bp, Homo sapiens
aaggtgatggaagaaacactc

SEQ ID NO: 826 - RNA, 21 nt, RNAi-sense strand
ggugauggaagaaacacucuu

SEQ ID NO: 827 - RNA, 21 nt, RNAi-antisense strand
gaguguuucuuccaucaccuu

SEQ ID NO: 828 - DNA, 21 bp, Homo sapiens
aagaaacactcagctatctac

SEQ ID NO: 829 - RNA, 21 nt, RNAi-sense strand
gaaacacucagcuaucuacuu

SEQ ID NO: 830 - RNA, 21 nt, RNAi-antisense strand
guagauagcugaguguuucuu

SEQ ID NO: 831 - DNA, 21 bp, Homo sapiens
aaacactcagctatctactag

SEQ ID NO: 832 - RNA, 21 nt, RNAi-sense strand
acacucagcuaucuacuaguu

SEQ ID NO: 833 - RNA, 21 nt, RNAi-antisense strand
cuaguagauagcugaguguuu

SEQ ID NO: 834 - DNA, 21 bp, Homo sapiens
aacactcagctatctactagg

SEQ ID NO: 835 - RNA, 21 nt, RNAi-sense strand
cacucagcuaucuacuagguu

SEQ ID NO: 836 - RNA, 21 nt, RNAi-antisense strand
ccuaguagauagcugaguguu

SEQ ID NO: 837 - DNA, 21 bp, Homo sapiens
aaaggccaattcatctaagtt

SEQ ID NO: 838 - RNA, 21 nt, RNAi-sense strand
aggccaauucaucuaaguuuu

SEQ ID NO: 839 - RNA, 21 nt, RNAi-antisense strand
aacuuagaugaauuggccuuu

SEQ ID NO: 840 - DNA, 21 bp, Homo sapiens
aaggccaattcatctaagttt

SEQ ID NO: 841 - RNA, 21 nt, RNAi-sense strand
ggccaauucaucuaaguuuuu

SEQ ID NO: 842 - RNA, 21 nt, RNAi-antisense strand
aaacuuagaugaauuggccuu

SEQ ID NO: 843 - DNA, 21 bp, Homo sapiens
aaggtctctacatcacagaag

SEQ ID NO: 844 - RNA, 21 nt, RNAi-sense strand
ggucucuacaucacagaaguu

SEQ ID NO: 845 - RNA, 21 nt, RNAi-antisense strand
cuucugugauguagagaccuu

SEQ ID NO: 846 - DNA, 21 bp, Homo sapiens
aagacaccagaagaagtaact

SEQ ID NO: 847 - RNA, 21 nt, RNAi-sense strand
gacaccagaagaaguaacuuu

SEQ ID NO: 848 - RNA, 21 nt, RNAi-antisense strand
aguuacuucuucuggugucuu

SEQ ID NO: 849 - DNA, 21 bp, Homo sapiens
aagaagtaactcgaacagtga

SEQ ID NO: 850 - RNA, 21 nt, RNAi-sense strand
gaaguaacucgaacagugauu

SEQ ID NO: 851 - RNA, 21 nt, RNAi-antisense strand
ucacuguucgaguuacuucuu

SEQ ID NO: 852 - DNA, 21 bp, Homo sapiens
aagtaactcgaacagtgaaca

SEQ ID NO: 853 - RNA, 21 nt, RNAi-sense strand
guaacucgaacagugaacauu

SEQ ID NO: 854 - RNA, 21 nt, RNAi-antisense strand
uguucacuguucgaguuacuu

SEQ ID NO: 855 - DNA, 21 bp, Homo sapiens
aactcgaacagtgaacacagc

SEQ ID NO: 856 - RNA, 21 nt, RNAi-sense strand
cucgaacagugaacacagcuu

SEQ ID NO: 857 - RNA, 21 nt, RNAi-antisense strand
gcuguguucacuguucgaguu

SEQ ID NO: 858 - DNA, 21 bp, Homo sapiens
aacagtgaacacagcagttgc

SEQ ID NO: 859 - RNA, 21 nt, RNAi-sense strand
cagugaacacagcaguugcuu

SEQ ID NO: 860 - RNA, 21 nt, RNAi-antisense strand
gcaacugcuguguucacuguu

SEQ ID NO: 861 - DNA, 21 bp, Homo sapiens
aacacagcagttgcaataacc

SEQ ID NO: 862 - RNA, 21 nt, RNAi-sense strand
cacagcaguugcaauaaccuu

SEQ ID NO: 863 - RNA, 21 nt, RNAi-antisense strand
gguuauugcaacugcuguguu

SEQ ID NO: 864 - DNA, 21 bp, Homo sapiens
aataaccttggcttgtttcgg

SEQ ID NO: 865 - RNA, 21 nt, RNAi-sense strand
uaaccuuggcuuguuucgguu

SEQ ID NO: 866 - RNA, 21 nt, RNAi-antisense strand
ccgaaacaagccaagguuauu

SEQ ID NO: 867 - DNA, 21 bp, Homo sapiens
aaccttggcttgtttcggact

SEQ ID NO: 868 - RNA, 21 nt, RNAi-sense strand
ccuuggcuuguuucggacuuu

SEQ ID NO: 869 - RNA, 21 nt, RNAi-antisense strand
aguccgaaacaagccaagguu

SEQ ID NO: 870 - DNA, 21 bp, Homo sapiens
aatcacaagcctattgactac

SEQ ID NO: 871 - RNA, 21 nt, RNAi-sense strand
ucacaagccuauugacuacuu

SEQ ID NO: 872 - RNA, 21 nt, RNAi-antisense strand
guagucaauaggcuugugauu

SEQ ID NO: 873 - DNA, 21 bp, Homo sapiens
aagcctattgactaccttaac

SEQ ID NO: 874 - RNA, 21 nt, RNAi-sense strand
gccuauugacuaccuuaacuu

SEQ ID NO: 875 - RNA, 21 nt, RNAi-antisense strand
guuaagguagucaauaggcuu

SEQ ID NO: 876 - DNA, 21 bp, Homo sapiens
aagtaaatgtggaaacatccg

SEQ ID NO: 877 - RNA, 21 nt, RNAi-sense strand
guaaauguggaaacauccguu

SEQ ID NO: 878 - RNA, 21 nt, RNAi-antisense strand
cggauguuuccacauuuacuu

SEQ ID NO: 879 - DNA, 21 bp, Homo sapiens
aaatgtggaaacatccgatat

SEQ ID NO: 880 - RNA, 21 nt, RNAi-sense strand
auguggaaacauccgauauuu

SEQ ID NO: 881 - RNA, 21 nt, RNAi-antisense strand
auaucggauguuuccacauuu

SEQ ID NO: 882 - DNA, 21 bp, Homo sapiens
aatgtggaaacatccgatata

SEQ ID NO: 883 - RNA, 21 nt, RNAi-sense strand
uguggaaacauccgauauauu

SEQ ID NO: 884 - RNA, 21 nt, RNAi-antisense strand
uauaucggauguuuccacauu

SEQ ID NO: 885 - DNA, 21 bp, Homo sapiens
aagcatagagttatccttcta

SEQ ID NO: 886 - RNA, 21 nt, RNAi-sense strand
gcauagaguuauccuucuauu

SEQ ID NO: 887 - RNA, 21 nt, RNAi-antisense strand
uagaaggauaacucuaugcuu

SEQ ID NO: 888 - DNA, 21 bp, Homo sapiens
aagacttatccttagaaagag

SEQ ID NO: 889 - RNA, 21 nt, RNAi-sense strand
gacuuauccuuagaaagaguu

SEQ ID NO: 890 - RNA, 21 nt, RNAi-antisense strand
cucuuucuaaggauaagucuu

SEQ ID NO: 891 - DNA, 21 bp, Homo sapiens
aataagcacacttacataagc

SEQ ID NO: 892 - RNA, 21 nt, RNAi-sense strand
uaagcacacuuacauaagcuu

SEQ ID NO: 893 - RNA, 21 nt, RNAi-antisense strand
gcuuauguaagugugcuuauu

SEQ ID NO: 894 - DNA, 21 bp, Homo sapiens
aatcaggagacaaagctacca

SEQ ID NO: 895 - RNA, 21 nt, RNAi-sense strand
ucaggagacaaagcuaccauu

SEQ ID NO: 896 - RNA, 21 nt, RNAi-antisense strand
ugguagcuuugucuccugauu

SEQ ID NO: 897 - DNA, 21 bp, Homo sapiens
aaagctaccacatgtggaaag

SEQ ID NO: 898 - RNA, 21 nt, RNAi-sense strand
agcuaccacauguggaaaguu

SEQ ID NO: 899 - RNA, 21 nt, RNAi-antisense strand
cuuuccacaugugguagcuuu

SEQ ID NO: 900 - DNA, 21 bp, Homo sapiens
aagctaccacatgtggaaagg

SEQ ID NO: 901 - RNA, 21 nt, RNAi-sense strand
gcuaccacauguggaaagguu

SEQ ID NO: 902 - RNA, 21 nt, RNAi-antisense strand
ccuuuccacaugugguagcuu

SEQ ID NO: 903 - DNA, 21 bp, Homo sapiens
aaaggtactatgtgtccatgt

SEQ ID NO: 904 - RNA, 21 nt, RNAi-sense strand
agguacuauguguccauguuu

SEQ ID NO: 905 - RNA, 21 nt, RNAi-antisense strand
acauggacacauaguaccuuu

SEQ ID NO: 906 - DNA, 21 bp, Homo sapiens
aaggtactatgtgtccatgtc

SEQ ID NO: 907 - RNA, 21 nt, RNAi-sense strand
gguacuauguguccaugucuu

SEQ ID NO: 908 - RNA, 21 nt, RNAi-antisense strand
gacauggacacauaguaccuu

SEQ ID NO: 909 - DNA, 21 bp, Homo sapiens
caagtgcagcaaagagaagtg

SEQ ID NO: 910 - RNA, 21 nt, RNAi-sense strand
agugcagcaaagagaaguguu

SEQ ID NO: 911 - RNA, 21 nt, RNAi-antisense strand
cacuucucuuugcugcacuuu

SEQ ID NO: 912 - DNA, 21 bp, Homo sapiens
cagcaaagagaagtgtcagag

SEQ ID NO: 913 - RNA, 21 nt, RNAi-sense strand
gcaaagagaagugucagaguu

SEQ ID NO: 914 - RNA, 21 nt, RNAi-antisense strand
cucugacacuucucuuugcuu

SEQ ID NO: 915 - DNA, 21 bp, Homo sapiens
caaagagaagtgtcagagcat

SEQ ID NO: 916 - RNA, 21 nt, RNAi-sense strand
aagagaagugucagagcauuu

SEQ ID NO: 917 - RNA, 21 nt, RNAi-antisense strand
augcucugacacuucucuuuu

SEQ ID NO: 918 - DNA, 21 bp, Homo sapiens
cagagcatgagcgccaagtcc

SEQ ID NO: 919 - RNA, 21 nt, RNAi-sense strand
gagcaugagcgccaaguccuu

SEQ ID NO: 920 - RNA, 21 nt, RNAi-antisense strand
ggacuuggcgcucaugcucuu

SEQ ID NO: 921 - DNA, 21 bp, Homo sapiens
catgagcgccaagtccagaac

SEQ ID NO: 922 - RNA, 21 nt, RNAi-sense strand
ugagcgccaaguccagaacuu

SEQ ID NO: 923 - RNA, 21 nt, RNAi-antisense strand
guucuggacuuggcgcucauu

SEQ ID NO: 924 - DNA, 21 bp, Homo sapiens
cagtattgagaaaggctggtc

SEQ ID NO: 925 - RNA, 21 nt, RNAi-sense strand
guauugagaaaggcuggucuu

SEQ ID NO: 926 - RNA, 21 nt, RNAi-antisense strand
gaccagccuuucucaauacuu

SEQ ID NO: 927 - DNA, 21 bp, Homo sapiens
caagagtgtgatgtgaaggat

SEQ ID NO: 928 - RNA, 21 nt, RNAi-sense strand
agagugugaugugaaggauuu

SEQ ID NO: 929 - RNA, 21 nt, RNAi-antisense strand
auccuucacaucacacucuuu

SEQ ID NO: 930 - DNA, 21 bp, Homo sapiens
caaattgtgaagaatccaagg

SEQ ID NO: 931 - RNA, 21 nt, RNAi-sense strand
aauugugaagaauccaagguu

SEQ ID NO: 932 - RNA, 21 nt, RNAi-antisense strand
ccuuggauucuucacaauuuu

SEQ ID NO: 933 - DNA, 21 bp, Homo sapiens
caaggtggcagaagtcaagaa

SEQ ID NO: 934 - RNA, 21 nt, RNAi-sense strand
agguggcagaagucaagaauu

SEQ ID NO: 935 - RNA, 21 nt, RNAi-antisense strand
uucuugacuucugccaccuuu

SEQ ID NO: 936 - DNA, 21 bp, Homo sapiens
cagaagtcaagaagaacggaa

SEQ ID NO: 937 - RNA, 21 nt, RNAi-sense strand
gaagucaagaagaacggaauu

SEQ ID NO: 938 - RNA, 21 nt, RNAi-antisense strand
uuccguucuucuugacuucuu

SEQ ID NO: 939 - DNA, 21 bp, Homo sapiens
caagaagaacggaagaatcag

SEQ ID NO: 940 - RNA, 21 nt, RNAi-sense strand
agaagaacggaagaaucaguu

SEQ ID NO: 941 - RNA, 21 nt, RNAi-antisense strand
cugauucuuccguucuucuuu

SEQ ID NO: 942 - DNA, 21 bp, Homo sapiens
cacagtttggcaattggaagc

SEQ ID NO: 943 - RNA, 21 nt, RNAi-sense strand
caguuuggcaauuggaagcuu

SEQ ID NO: 944 - RNA, 21 nt, RNAi-antisense strand
gcuuccaauugccaaacuguu

SEQ ID NO: 945 - DNA, 21 bp, Homo sapiens
cagtttggcaattggaagcat

SEQ ID NO: 946 - RNA, 21 nt, RNAi-sense strand
guuuggcaauuggaagcauuu

SEQ ID NO: 947 - RNA, 21 nt, RNAi-antisense strand
augcuuccaauugccaaacuu

SEQ ID NO: 948 - DNA, 21 bp, Homo sapiens
caattggaagcatctctggcc

SEQ ID NO: 949 - RNA, 21 nt, RNAi-sense strand
auuggaagcaucucuggccuu

SEQ ID NO: 950 - RNA, 21 nt, RNAi-antisense strand
ggccagagaugcuuccaauuu

SEQ ID NO: 951 - DNA, 21 bp, Homo sapiens
cacactgatatcaacactcca

SEQ ID NO: 952 - RNA, 21 nt, RNAi-sense strand
cacugauaucaacacuccauu

SEQ ID NO: 953 - RNA, 21 nt, RNAi-antisense strand
uggaguguugauaucaguguu

SEQ ID NO: 954 - DNA, 21 bp, Homo sapiens
cactgatatcaacactccact

SEQ ID NO: 955 - RNA, 21 nt, RNAi-sense strand
cugauaucaacacuccacuuu

SEQ ID NO: 956 - RNA, 21 nt, RNAi-antisense strand
aguggaguguugauaucaguu

SEQ ID NO: 957 - DNA, 21 bp, Homo sapiens
caacactccactgacaaccac

SEQ ID NO: 958 - RNA, 21 nt, RNAi-sense strand
acacuccacugacaaccacuu

SEQ ID NO: 959 - RNA, 21 nt, RNAi-antisense strand
gugguugucaguggaguguuu

SEQ ID NO: 960 - DNA, 21 bp, Homo sapiens
cactccactgacaaccacaag

SEQ ID NO: 961 - RNA, 21 nt, RNAi-sense strand
cuccacugacaaccacaaguu

SEQ ID NO: 962 - RNA, 21 nt, RNAi-antisense strand
cuugugguugucaguggaguu

SEQ ID NO: 963 - DNA, 21 bp, Homo sapiens
cactgacaaccacaagtggaa

SEQ ID NO: 964 - RNA, 21 nt, RNAi-sense strand
cugacaaccacaaguggaauu

SEQ ID NO: 965 - RNA, 21 nt, RNAi-antisense strand
uuccacuugugguugucaguu

SEQ ID NO: 966 - DNA, 21 bp, Homo sapiens
caaccacaagtggaaacttgc

SEQ ID NO: 967 - RNA, 21 nt, RNAi-sense strand
accacaaguggaaacuugcuu

SEQ ID NO: 968 - RNA, 21 nt, RNAi-antisense strand
gcaaguuuccacuugugguuu

SEQ ID NO: 969 - DNA, 21 bp, Homo sapiens
cacaagtggaaacttgcatgg

SEQ ID NO: 970 - RNA, 21 nt, RNAi-sense strand
caaguggaaacuugcaugguu

SEQ ID NO: 971 - RNA, 21 nt, RNAi-antisense strand
ccaugcaaguuuccacuuguu

SEQ ID NO: 972 - DNA, 21 bp, Homo sapiens
caagtggaaacttgcatggac

SEQ ID NO: 973 - RNA, 21 nt, RNAi-sense strand
aguggaaacuugcauggacuu

SEQ ID NO: 974 - RNA, 21 nt, RNAi-antisense strand
guccaugcaaguuuccacuuu

SEQ ID NO: 975 - DNA, 21 bp, Homo sapiens
catggacaacctgtatctttc

SEQ ID NO: 976 - RNA, 21 nt, RNAi-sense strand
uggacaaccuguaucuuucuu

SEQ ID NO: 977 - RNA, 21 nt, RNAi-antisense strand
gaaagauacagguuguccauu

SEQ ID NO: 978 - DNA, 21 bp, Homo sapiens
caacctgtatctttcctcctg

SEQ ID NO: 979 - RNA, 21 nt, RNAi-sense strand
accuguaucuuuccuccuguu

SEQ ID NO: 980 - RNA, 21 nt, RNAi-antisense strand
caggaggaaagauacagguuu

SEQ ID NO: 981 - DNA, 21 bp, Homo sapiens
caaggatattgtgtatattgg

SEQ ID NO: 982 - RNA, 21 nt, RNAi-sense strand
aggauauuguguauauugguu

SEQ ID NO: 983 - RNA, 21 nt, RNAi-antisense strand
ccaauauacacaauauccuuu

SEQ ID NO: 984 - DNA, 21 bp, Homo sapiens
caatgactgaagtggacagac

SEQ ID NO: 985 - RNA, 21 nt, RNAi-sense strand
augacugaaguggacagacuu

SEQ ID NO: 986 - RNA, 21 nt, RNAi-antisense strand
gucuguccacuucagucauuu

SEQ ID NO: 987 - DNA, 21 bp, Homo sapiens
cagactaggaattggcaaggt

SEQ ID NO: 988 - RNA, 21 nt, RNAi-sense strand
gacuaggaauuggcaagguuu

SEQ ID NO: 989 - RNA, 21 nt, RNAi-antisense strand
accuugccaauuccuagucuu

SEQ ID NO: 990 - DNA, 21 bp, Homo sapiens
caaggtgatggaagaaacact

SEQ ID NO: 991 - RNA, 21 nt, RNAi-sense strand
aggugauggaagaaacacuuu

SEQ ID NO: 992 - RNA, 21 nt, RNAi-antisense strand
aguguuucuuccaucaccuuu

SEQ ID NO: 993 - DNA, 21 bp, Homo sapiens
cactcagctatctactaggaa

SEQ ID NO: 994 - RNA, 21 nt, RNAi-sense strand
cucagcuaucuacuaggaauu

SEQ ID NO: 995 - RNA, 21 nt, RNAi-antisense strand
uuccuaguagauagcugaguu

SEQ ID NO: 996 - DNA, 21 bp, Homo sapiens
cagctatctactaggaagaaa

SEQ ID NO: 997 - RNA, 21 nt, RNAi-sense strand
gcuaucuacuaggaagaaauu

SEQ ID NO: 998 - RNA, 21 nt, RNAi-antisense strand
uuucuuccuaguagauagcuu

SEQ ID NO: 999 - DNA, 21 bp, Homo sapiens
catctttcacaccagctactg

SEQ ID NO: 1000 - RNA, 21 nt, RNAi-sense strand
ucuuucacaccagcuacuguu

SEQ ID NO: 1001 - RNA, 21 nt, RNAi-antisense strand
caguagcuggugugaaagauu

SEQ ID NO: 1002 - DNA, 21 bp, Homo sapiens
cacaccagctactggcacacc

SEQ ID NO: 1003 - RNA, 21 nt, RNAi-sense strand
caccagcuacuggcacaccuu

SEQ ID NO: 1004 - RNA, 21 nt, RNAi-antisense strand
ggugugccaguagcugguguu

SEQ ID NO: 1005 - DNA, 21 bp, Homo sapiens
caccagctactggcacaccag

SEQ ID NO: 1006 - RNA, 21 nt, RNAi-sense strand
ccagcuacuggcacaccaguu

SEQ ID NO: 1007 - RNA, 21 nt, RNAi-antisense strand
cuggugugccaguagcugguu

SEQ ID NO: 1008 - DNA, 21 bp, Homo sapiens
cagctactggcacaccagtcg

SEQ ID NO: 1009 - RNA, 21 nt, RNAi-sense strand
gcuacuggcacaccagucguu

SEQ ID NO: 1010 - RNA, 21 nt, RNAi-antisense strand
cgacuggugugccaguagcuu

SEQ ID NO: 1011 - DNA, 21 bp, Homo sapiens
catacagagaaggtctctaca

SEQ ID NO: 1012 - RNA, 21 nt, RNAi-sense strand
uacagagaaggucucuacauu

SEQ ID NO: 1013 - RNA, 21 nt, RNAi-antisense strand
uguagagaccuucucuguauu

SEQ ID NO: 1014 - DNA, 21 bp, Homo sapiens
cagagaaggtctctacatcac

SEQ ID NO: 1015 - RNA, 21 nt, RNAi-sense strand
gagaaggucucuacaucacuu

SEQ ID NO: 1016 - RNA, 21 nt, RNAi-antisense strand
gugauguagagaccuucucuu

SEQ ID NO: 1017 - DNA, 21 bp, Homo sapiens
catcacagaagaaatctacaa

SEQ ID NO: 1018 - RNA, 21 nt, RNAi-sense strand
ucacagaagaaaucuacaauu

SEQ ID NO: 1019 - RNA, 21 nt, RNAi-antisense strand
uuguagauuucuucugugauu

SEQ ID NO: 1020 - DNA, 21 bp, Homo sapiens
caggattagatataatggaag

SEQ ID NO: 1021 - RNA, 21 nt, RNAi-sense strand
ggauuagauauaauggaaguu

SEQ ID NO: 1022 - RNA, 21 nt, RNAi-antisense strand
cuuccauuauaucuaauccuu

SEQ ID NO: 1023 - DNA, 21 bp, Homo sapiens
caccagaagaagtaactcgaa

SEQ ID NO: 1024 - RNA, 21 nt, RNAi-sense strand
ccagaagaaguaacucgaauu

SEQ ID NO: 1025 - RNA, 21 nt, RNAi-antisense strand
uucgaguuacuucuucugguu

SEQ ID NO: 1026 - DNA, 21 bp, Homo sapiens
cagaagaagtaactcgaacag

SEQ ID NO: 1027 - RNA, 21 nt, RNAi-sense strand
gaagaaguaacucgaacaguu

SEQ ID NO: 1028 - RNA, 21 nt, RNAi-antisense strand
cuguucgaguuacuucuucuu

SEQ ID NO: 1029 - DNA, 21 bp, Homo sapiens
cagtgaacacagcagttgcaa

SEQ ID NO: 1030 - RNA, 21 nt, RNAi-sense strand
gugaacacagcaguugcaauu

SEQ ID NO: 1031 - RNA, 21 nt, RNAi-antisense strand
uugcaacugcuguguucacuu

SEQ ID NO: 1032 - DNA, 21 bp, Homo sapiens
cacagcagttgcaataacctt

SEQ ID NO: 1033 - RNA, 21 nt, RNAi-sense strand
cagcaguugcaauaaccuuuu

SEQ ID NO: 1034 - RNA, 21 nt, RNAi-antisense strand
aagguuauugcaacugcuguu

SEQ ID NO: 1035 - DNA, 21 bp, Homo sapiens
cagcagttgcaataaccttgg

SEQ ID NO: 1036 - RNA, 21 nt, RNAi-sense strand
gcaguugcaauaaccuugguu

SEQ ID NO: 1037 - RNA, 21 nt, RNAi-antisense strand
ccaagguuauugcaacugcuu

SEQ ID NO: 1038 - DNA, 21 bp, Homo sapiens
cagttgcaataaccttggctt

SEQ ID NO: 1039 - RNA, 21 nt, RNAi-sense strand
guugcaauaaccuuggcuuuu

SEQ ID NO: 1040 - RNA, 21 nt, RNAi-antisense strand
aagccaagguuauugcaacuu

SEQ ID NO: 1041 - DNA, 21 bp, Homo sapiens
caataaccttggcttgtttcg

SEQ ID NO: 1042 - RNA, 21 nt, RNAi-sense strand
auaaccuuggcuuguuucguu

SEQ ID NO: 1043 - RNA, 21 nt, RNAi-antisense strand
cgaaacaagccaagguuauuu

SEQ ID NO: 1044 - DNA, 21 bp, Homo sapiens
cacaagcctattgactacctt

SEQ ID NO: 1045 - RNA, 21 nt, RNAi-sense strand
caagccuauugacuaccuuuu

SEQ ID NO: 1046 - RNA, 21 nt, RNAi-antisense strand
aagguagucaauaggcuuguu

SEQ ID NO: 1047 - DNA, 21 bp, Homo sapiens
caagcctattgactaccttaa

SEQ ID NO: 1048 - RNA, 21 nt, RNAi-sense strand
agccuauugacuaccuuaauu

SEQ ID NO: 1049 - RNA, 21 nt, RNAi-antisense strand
uuaagguagucaauaggcuuu

SEQ ID NO: 1050 - DNA, 21 bp, Homo sapiens
cacctaagtaaatgtggaaac

SEQ ID NO: 1051 - RNA, 21 nt, RNAi-sense strand
ccuaaguaaauguggaaacuu

SEQ ID NO: 1052 - RNA, 21 nt, RNAi-antisense strand
guuuccacauuuacuuagguu

SEQ ID NO: 1053 - DNA, 21 bp, Homo sapiens
catccgatataaatctcatag

SEQ ID NO: 1054 - RNA, 21 nt, RNAi-sense strand
uccgauauaaaucucauaguu

SEQ ID NO: 1055 - RNA, 21 nt, RNAi-antisense strand
cuaugagauuuauaucggauu

SEQ ID NO: 1056 - DNA, 21 bp, Homo sapiens
catagagttatccttctaaag

SEQ ID NO: 1057 - RNA, 21 nt, RNAi-sense strand
uagaguuauccuucuaaaguu

SEQ ID NO: 1058 - RNA, 21 nt, RNAi-antisense strand
cuuuagaaggauaacucuauu

SEQ ID NO: 1059 - DNA, 21 bp, Homo sapiens
caagatgtggaaattctaact

SEQ ID NO: 1060 - RNA, 21 nt, RNAi-sense strand
agauguggaaauucuaacuuu

SEQ ID NO: 1061 - RNA, 21 nt, RNAi-antisense strand
aguuagaauuuccacaucuuu

SEQ ID NO: 1062 - DNA, 21 bp, Homo sapiens
caggagacaaagctaccacat

SEQ ID NO: 1063 - RNA, 21 nt, RNAi-sense strand
ggagacaaagcuaccacauuu

SEQ ID NO: 1064 - RNA, 21 nt, RNAi-antisense strand
augugguagcuuugucuccuu

SEQ ID NO: 1065 - DNA, 21 bp, Homo sapiens
caaagctaccacatgtggaaa

SEQ ID NO: 1066 - RNA, 21 nt, RNAi-sense strand
aagcuaccacauguggaaauu

SEQ ID NO: 1067 - RNA, 21 nt, RNAi-antisense strand
uuuccacaugugguagcuuuu

SEQ ID NO: 1068 - DNA, 21 bp, Homo sapiens
cacatgtggaaaggtactatg

SEQ ID NO: 1069 - RNA, 21 nt, RNAi-sense strand
cauguggaaagguacuauguu

SEQ ID NO: 1070 - RNA, 21 nt, RNAi-antisense strand
cauaguaccuuuccacauguu

SEQ ID NO: 1071 - DNA, 21 bp, Homo sapiens
catgtggaaaggtactatgtg

SEQ ID NO: 1072 - RNA, 21 nt, RNAi-sense strand
uguggaaagguacuauguguu

SEQ ID NO: 1073 - RNA, 21 nt, RNAi-antisense strand
cacauaguaccuuuccacauu

SEQ ID NO: 1074 - DNA, 21 bp, Homo sapiens
gactgactggagagctcaagt

SEQ ID NO: 1075 - RNA, 21 nt, RNAi-sense strand
cugacuggagagcucaaguuu

SEQ ID NO: 1076 - RNA, 21 nt, RNAi-antisense strand
acuugagcucuccagucaguu

SEQ ID NO: 1077 - DNA, 21 bp, Homo sapiens
gactggagagctcaagtgcag

SEQ ID NO: 1078 - RNA, 21 nt, RNAi-sense strand
cuggagagcucaagugcaguu

SEQ ID NO: 1079 - RNA, 21 nt, RNAi-antisense strand
cugcacuugagcucuccaguu

SEQ ID NO: 1080 - DNA, 21 bp, Homo sapiens
gagagctcaagtgcagcaaag

SEQ ID NO: 1081 - RNA, 21 nt, RNAi-sense strand
gagcucaagugcagcaaaguu

SEQ ID NO: 1082 - RNA, 21 nt, RNAi-antisense strand
cuuugcugcacuugagcucuu

SEQ ID NO: 1083 - DNA, 21 bp, Homo sapiens
gagctcaagtgcagcaaagag

SEQ ID NO: 1084 - RNA, 21 nt, RNAi-sense strand
gcucaagugcagcaaagaguu

SEQ ID NO: 1085 - RNA, 21 nt, RNAi-antisense strand
cucuuugcugcacuugagcuu

SEQ ID NO: 1086 - DNA, 21 bp, Homo sapiens
gagaagtgtcagagcatgagc

SEQ ID NO: 1087 - RNA, 21 nt, RNAi-sense strand
gaagugucagagcaugagcuu

SEQ ID NO: 1088 - RNA, 21 nt, RNAi-antisense strand
gcucaugcucugacacuucuu

SEQ ID NO: 1089 - DNA, 21 bp, Homo sapiens
gaagtgtcagagcatgagcgc

SEQ ID NO: 1090 - RNA, 21 nt, RNAi-sense strand
agugucagagcaugagcgcuu

SEQ ID NO: 1091 - RNA, 21 nt, RNAi-antisense strand
gcgcucaugcucugacacuuu

SEQ ID NO: 1092 - DNA, 21 bp, Homo sapiens
gagcatgagcgccaagtccag

SEQ ID NO: 1093 - RNA, 21 nt, RNAi-sense strand
gcaugagcgccaaguccaguu

SEQ ID NO: 1094 - RNA, 21 nt, RNAi-antisense strand
cuggacuuggcgcucaugcuu

SEQ ID NO: 1095 - DNA, 21 bp, Homo sapiens
gagcgccaagtccagaaccat

SEQ ID NO: 1096 - RNA, 21 nt, RNAi-sense strand
gcgccaaguccagaaccauuu

SEQ ID NO: 1097 - RNA, 21 nt, RNAi-antisense strand
augguucuggacuuggcgcuu

SEQ ID NO: 1098 - DNA, 21 bp, Homo sapiens
gattattggagctcctttctc

SEQ ID NO: 1099 - RNA, 21 nt, RNAi-sense strand
uuauuggagcuccuuucucuu

SEQ ID NO: 1100 - RNA, 21 nt, RNAi-antisense strand
gagaaaggagcuccaauaauu

SEQ ID NO: 1101 - DNA, 21 bp, Homo sapiens
gagaaaggctggtctgcttga

SEQ ID NO: 1102 - RNA, 21 nt, RNAi-sense strand
gaaaggcuggucugcuugauu

SEQ ID NO: 1103 - RNA, 21 nt, RNAi-antisense strand
ucaagcagaccagccuuucuu

SEQ ID NO: 1104 - DNA, 21 bp, Homo sapiens
gaaaggctggtctgcttgaga

SEQ ID NO: 1105 - RNA, 21 nt, RNAi-sense strand
aaggcuggucugcuugagauu

SEQ ID NO: 1106 - RNA, 21 nt, RNAi-antisense strand
ucucaagcagaccagccuuuu

SEQ ID NO: 1107 - DNA, 21 bp, Homo sapiens
gagaaacttaaagaacaagag

SEQ ID NO: 1108 - RNA, 21 nt, RNAi-sense strand
gaaacuuaaagaacaagaguu

SEQ ID NO: 1109 - RNA, 21 nt, RNAi-antisense strand
cucuuguucuuuaaguuucuu

SEQ ID NO: 1110 - DNA, 21 bp, Homo sapiens
gaaacttaaagaacaagagtg

SEQ ID NO: 1111 - RNA, 21 nt, RNAi-sense strand
aacuuaaagaacaagaguguu

SEQ ID NO: 1112 - RNA, 21 nt, RNAi-antisense strand
cacucuuguucuuuaaguuuu

SEQ ID NO: 1113 - DNA, 21 bp, Homo sapiens
gaacaagagtgtgatgtgaag

SEQ ID NO: 1114 - RNA, 21 nt, RNAi-sense strand
acaagagugugaugugaaguu

SEQ ID NO: 1115 - RNA, 21 nt, RNAi-antisense strand
cuucacaucacacucuuguuu

SEQ ID NO: 1116 - DNA, 21 bp, Homo sapiens
gagtgtgatgtgaaggattat

SEQ ID NO: 1117 - RNA, 21 nt, RNAi-sense strand
gugugaugugaaggauuauuu

SEQ ID NO: 1118 - RNA, 21 nt, RNAi-antisense strand
auaauccuucacaucacacuu

SEQ ID NO: 1119 - DNA, 21 bp, Homo sapiens
gaagtcaagaagaacggaaga

SEQ ID NO: 1120 - RNA, 21 nt, RNAi-sense strand
agucaagaagaacggaagauu

SEQ ID NO: 1121 - RNA, 21 nt, RNAi-antisense strand
ucuuccguucuucuugacuuu

SEQ ID NO: 1122 - DNA, 21 bp, Homo sapiens
gaagaacggaagaatcagcct

SEQ ID NO: 1123 - RNA, 21 nt, RNAi-sense strand
agaacggaagaaucagccuuu

SEQ ID NO: 1124 - RNA, 21 nt, RNAi-antisense strand
aggcugauucuuccguucuuu

SEQ ID NO: 1125 - DNA, 21 bp, Homo sapiens
gaacggaagaatcagcctggt

SEQ ID NO: 1126 - RNA, 21 nt, RNAi-sense strand
acggaagaaucagccugguuu

SEQ ID NO: 1127 - RNA, 21 nt, RNAi-antisense strand
accaggcugauucuuccguuu

SEQ ID NO: 1128 - DNA, 21 bp, Homo sapiens
gaagaatcagcctggtgctgg

SEQ ID NO: 1129 - RNA, 21 nt, RNAi-sense strand
agaaucagccuggugcugguu

SEQ ID NO: 1130 - RNA, 21 nt, RNAi-antisense strand
ccagcaccaggcugauucuuu

SEQ ID NO: 1131 - DNA, 21 bp, Homo sapiens
gagaccacagtttggcaattg

SEQ ID NO: 1132 - RNA, 21 nt, RNAi-sense strand
gaccacaguuuggcaauuguu

SEQ ID NO: 1133 - RNA, 21 nt, RNAi-antisense strand
caauugccaaacuguggucuu

SEQ ID NO: 1134 - DNA, 21 bp, Homo sapiens
gaccacagtttggcaattgga

SEQ ID NO: 1135 - RNA, 21 nt, RNAi-sense strand
ccacaguuuggcaauuggauu

SEQ ID NO: 1136 - RNA, 21 nt, RNAi-antisense strand
uccaauugccaaacugugguu

SEQ ID NO: 1137 - DNA, 21 bp, Homo sapiens
gaagcatctctggccatgcca

SEQ ID NO: 1138 - RNA, 21 nt, RNAi-sense strand
agcaucucuggccaugccauu

SEQ ID NO: 1139 - RNA, 21 nt, RNAi-antisense strand
uggcauggccagagaugcuuu

SEQ ID NO: 1140 - DNA, 21 bp, Homo sapiens
gatgctcacactgatatcaac

SEQ ID NO: 1141 - RNA, 21 nt, RNAi-sense strand
ugcucacacugauaucaacuu

SEQ ID NO: 1142 - RNA, 21 nt, RNAi-antisense strand
guugauaucagugugagcauu

SEQ ID NO: 1143 - DNA, 21 bp, Homo sapiens
gatatcaacactccactgaca

SEQ ID NO: 1144 - RNA, 21 nt, RNAi-sense strand
uaucaacacuccacugacauu

SEQ ID NO: 1145 - RNA, 21 nt, RNAi-antisense strand
ugucaguggaguguugauauu

SEQ ID NO: 1146 - DNA, 21 bp, Homo sapiens
gacaaccacaagtggaaactt

SEQ ID NO: 1147 - RNA, 21 nt, RNAi-sense strand
caaccacaaguggaaacuuuu

SEQ ID NO: 1148 - RNA, 21 nt, RNAi-antisense strand
aaguuuccacuugugguuguu

SEQ ID NO: 1149 - DNA, 21 bp, Homo sapiens
gaaacttgcatggacaacctg

SEQ ID NO: 1150 - RNA, 21 nt, RNAi-sense strand
aacuugcauggacaaccuguu

SEQ ID NO: 1151 - RNA, 21 nt, RNAi-antisense strand
cagguuguccaugcaaguuuu

SEQ ID NO: 1152 - DNA, 21 bp, Homo sapiens
gacaacctgtatctttcctcc

SEQ ID NO: 1153 - RNA, 21 nt, RNAi-sense strand
caaccuguaucuuuccuccuu

SEQ ID NO: 1154 - RNA, 21 nt, RNAi-antisense strand
ggaggaaagauacagguuguu

SEQ ID NO: 1155 - DNA, 21 bp, Homo sapiens
gatgtgccaggattctcctgg

SEQ ID NO: 1156 - RNA, 21 nt, RNAi-sense strand
ugugccaggauucuccugguu

SEQ ID NO: 1157 - RNA, 21 nt, RNAi-antisense strand
ccaggagaauccuggcacauu

SEQ ID NO: 1158 - DNA, 21 bp, Homo sapiens
gatattgtgtatattggcttg

SEQ ID NO: 1159 - RNA, 21 nt, RNAi-sense strand
uauuguguauauuggcuuguu

SEQ ID NO: 1160 - RNA, 21 nt, RNAi-antisense strand
caagccaauauacacaauauu

SEQ ID NO: 1161 - DNA, 21 bp, Homo sapiens
gactgaagtggacagactagg

SEQ ID NO: 1162 - RNA, 21 nt, RNAi-sense strand
cugaaguggacagacuagguu

SEQ ID NO: 1163 - RNA, 21 nt, RNAi-antisense strand
ccuagucuguccacuucaguu

SEQ ID NO: 1164 - DNA, 21 bp, Homo sapiens
gaagtggacagactaggaatt

SEQ ID NO: 1165 - RNA, 21 nt, RNAi-sense strand
aguggacagacuaggaauuuu

SEQ ID NO: 1166 - RNA, 21 nt, RNAi-antisense strand
aauuccuagucuguccacuuu

SEQ ID NO: 1167 - DNA, 21 bp, Homo sapiens
gacagactaggaattggcaag

SEQ ID NO: 1168 - RNA, 21 nt, RNAi-sense strand
cagacuaggaauuggcaaguu

SEQ ID NO: 1169 - RNA, 21 nt, RNAi-antisense strand
cuugccaauuccuagucuguu

SEQ ID NO: 1170 - DNA, 21 bp, Homo sapiens
gactaggaattggcaaggtga

SEQ ID NO: 1171 - RNA, 21 nt, RNAi-sense strand
cuaggaauuggcaaggugauu

SEQ ID NO: 1172 - RNA, 21 nt, RNAi-antisense strand
ucaccuugccaauuccuaguu

SEQ ID NO: 1173 - DNA, 21 bp, Homo sapiens
gaattggcaaggtgatggaag

SEQ ID NO: 1174 - RNA, 21 nt, RNAi-sense strand
auuggcaaggugauggaaguu

SEQ ID NO: 1175 - RNA, 21 nt, RNAi-antisense strand
cuuccaucaccuugccaauuu

SEQ ID NO: 1176 - DNA, 21 bp, Homo sapiens
gatggaagaaacactcagcta

SEQ ID NO: 1177 - RNA, 21 nt, RNAi-sense strand
uggaagaaacacucagcuauu

SEQ ID NO: 1178 - RNA, 21 nt, RNAi-antisense strand
uagcugaguguuucuuccauu

SEQ ID NO: 1179 - DNA, 21 bp, Homo sapiens
gaagaaacactcagctatcta

SEQ ID NO: 1180 - RNA, 21 nt, RNAi-sense strand
agaaacacucagcuaucuauu

SEQ ID NO: 1181 - RNA, 21 nt, RNAi-antisense strand
uagauagcugaguguuucuuu

SEQ ID NO: 1182 - DNA, 21 bp, Homo sapiens
gaaacactcagctatctacta

SEQ ID NO: 1183 - RNA, 21 nt, RNAi-sense strand
aacacucagcuaucuacuauu

SEQ ID NO: 1184 - RNA, 21 nt, RNAi-antisense strand
uaguagauagcugaguguuuu

SEQ ID NO: 1185 - DNA, 21 bp, Homo sapiens
gaggtctgacatacagagaag

SEQ ID NO: 1186 - RNA, 21 nt, RNAi-sense strand
ggucugacauacagagaaguu

SEQ ID NO: 1187 - RNA, 21 nt, RNAi-antisense strand
cuucucuguaugucagaccuu

SEQ ID NO: 1188 - DNA, 21 bp, Homo sapiens
gacatacagagaaggtctcta

SEQ ID NO: 1189 - RNA, 21 nt, RNAi-sense strand
cauacagagaaggucucuauu

SEQ ID NO: 1190 - RNA, 21 nt, RNAi-antisense strand
uagagaccuucucuguauguu

SEQ ID NO: 1191 - DNA, 21 bp, Homo sapiens
gagaaggtctctacatcacag

SEQ ID NO: 1192 - RNA, 21 nt, RNAi-sense strand
gaaggucucuacaucacaguu

SEQ ID NO: 1193 - RNA, 21 nt, RNAi-antisense strand
cugugauguagagaccuucuu

SEQ ID NO: 1194 - DNA, 21 bp, Homo sapiens
gaaggtctctacatcacagaa

SEQ ID NO: 1195 - RNA, 21 nt, RNAi-sense strand
aggucucuacaucacagaauu

SEQ ID NO: 1196 - RNA, 21 nt, RNAi-antisense strand
uucugugauguagagaccuuu

SEQ ID NO: 1197 - DNA, 21 bp, Homo sapiens
gaagacaccagaagaagtaac

SEQ ID NO: 1198 - RNA, 21 nt, RNAi-sense strand
agacaccagaagaaguaacuu

SEQ ID NO: 1199 - RNA, 21 nt, RNAi-antisense strand
guuacuucuucuggugucuuu

SEQ ID NO: 1200 - DNA, 21 bp, Homo sapiens
gacaccagaagaagtaactcg

SEQ ID NO: 1201 - RNA, 21 nt, RNAi-sense strand
caccagaagaaguaacucguu

SEQ ID NO: 1202 - RNA, 21 nt, RNAi-antisense strand
cgaguuacuucuucugguguu

SEQ ID NO: 1203 - DNA, 21 bp, Homo sapiens
gaagaagtaactcgaacagtg

SEQ ID NO: 1204 - RNA, 21 nt, RNAi-sense strand
agaaguaacucgaacaguguu

SEQ ID NO: 1205 - RNA, 21 nt, RNAi-antisense strand
cacuguucgaguuacuucuuu

SEQ ID NO: 1206 - DNA, 21 bp, Homo sapiens
gaagtaactcgaacagtgaac

SEQ ID NO: 1207 - RNA, 21 nt, RNAi-sense strand
aguaacucgaacagugaacuu

SEQ ID NO: 1208 - RNA, 21 nt, RNAi-antisense strand
guucacuguucgaguuacuuu

SEQ ID NO: 1209 - DNA, 21 bp, Homo sapiens
gaacagtgaacacagcagttg

SEQ ID NO: 1210 - RNA, 21 nt, RNAi-sense strand
acagugaacacagcaguuguu

SEQ ID NO: 1211 - RNA, 21 nt, RNAi-antisense strand
caacugcuguguucacuguuu

SEQ ID NO: 1212 - DNA, 21 bp, Homo sapiens
gaacacagcagttgcaataac

SEQ ID NO: 1213 - RNA, 21 nt, RNAi-sense strand
acacagcaguugcaauaacuu

SEQ ID NO: 1214 - RNA, 21 nt, RNAi-antisense strand
guuauugcaacugcuguguuu

SEQ ID NO: 1215 - DNA, 21 bp, Homo sapiens
gaaacatccgatataaatctc

SEQ ID NO: 1216 - RNA, 21 nt, RNAi-sense strand
aacauccgauauaaaucucuu

SEQ ID NO: 1217 - RNA, 21 nt, RNAi-antisense strand
gagauuuauaucggauguuuu

SEQ ID NO: 1218 - DNA, 21 bp, Homo sapiens
gagttatccttctaaagactt

SEQ ID NO: 1219 - RNA, 21 nt, RNAi-sense strand
guuauccuucuaaagacuuuu

SEQ ID NO: 1220 - RNA, 21 nt, RNAi-antisense strand
aagucuuuagaaggauaacuu

SEQ ID NO: 1221 - DNA, 21 bp, Homo sapiens
gacttatccttagaaagagaa

SEQ ID NO: 1222 - RNA, 21 nt, RNAi-sense strand
cuuauccuuagaaagagaauu

SEQ ID NO: 1223 - RNA, 21 nt, RNAi-antisense strand
uucucuuucuaaggauaaguu

SEQ ID NO: 1224 - DNA, 21 bp, Homo sapiens
gaaagagaagtgtacattgat

SEQ ID NO: 1225 - RNA, 21 nt, RNAi-sense strand
aagagaaguguacauugauuu

SEQ ID NO: 1226 - RNA, 21 nt, RNAi-antisense strand
aucaauguacacuucucuuuu

SEQ ID NO: 1227 - DNA, 21 bp, Homo sapiens
gagaagtgtacattgatttcc

SEQ ID NO: 1228 - RNA, 21 nt, RNAi-sense strand
gaaguguacauugauuuccuu

SEQ ID NO: 1229 - RNA, 21 nt, RNAi-antisense strand
ggaaaucaauguacacuucuu

SEQ ID NO: 1230 - DNA, 21 bp, Homo sapiens
gaagtgtacattgatttccaa

SEQ ID NO: 1231 - RNA, 21 nt, RNAi-sense strand
aguguacauugauuuccaauu

SEQ ID NO: 1232 - RNA, 21 nt, RNAi-antisense strand
uuggaaaucaauguacacuuu

SEQ ID NO: 1233 - DNA, 21 bp, Homo sapiens
gactcttggaatcaggagaca

SEQ ID NO: 1234 - RNA, 21 nt, RNAi-sense strand
cucuuggaaucaggagacauu

SEQ ID NO: 1235 - RNA, 21 nt, RNAi-antisense strand
ugucuccugauuccaagaguu

SEQ ID NO: 1236 - DNA, 21 bp, Homo sapiens
gaatcaggagacaaagctacc

SEQ ID NO: 1237 - RNA, 21 nt, RNAi-sense strand
aucaggagacaaagcuaccuu

SEQ ID NO: 1238 - RNA, 21 nt, RNAi-antisense strand
gguagcuuugucuccugauuu

SEQ ID NO: 1239 - DNA, 21 bp, Homo sapiens
gagacaaagctaccacatgtg

SEQ ID NO: 1240 - RNA, 21 nt, RNAi-sense strand
gacaaagcuaccacauguguu

SEQ ID NO: 1241 - RNA, 21 nt, RNAi-antisense strand
cacaugugguagcuuugucuu

SEQ ID NO: 1242 - DNA, 21 bp, Homo sapiens
gacaaagctaccacatgtgga

SEQ ID NO: 1243 - RNA, 21 nt, RNAi-sense strand
caaagcuaccacauguggauu

SEQ ID NO: 1244 - RNA, 21 nt, RNAi-antisense strand
uccacaugugguagcuuuguu

SEQ ID NO: 1245 - DNA, 21 bp, Homo sapiens
gaaaggtactatgtgtccatg

SEQ ID NO: 1246 - RNA, 21 nt, RNAi-sense strand
aagguacuauguguccauguu

SEQ ID NO: 1247 - RNA, 21 nt, RNAi-antisense strand
cauggacacauaguaccuuuu

SEQ ID NO: 1248 - DNA, 21 bp, Homo sapiens
tattggagctcctttctcaaa

SEQ ID NO: 1249 - RNA, 21 nt, RNAi-sense strand
uuggagcuccuuucucaaauu

SEQ ID NO: 1250 - RNA, 21 nt, RNAi-antisense strand
uuugagaaaggagcuccaauu

SEQ ID NO: 1251 - DNA, 21 bp, Homo sapiens
tacagtattgagaaaggctgg

SEQ ID NO: 1252 - RNA, 21 nt, RNAi-sense strand
caguauugagaaaggcugguu

SEQ ID NO: 1253 - RNA, 21 nt, RNAi-antisense strand
ccagccuuucucaauacuguu

SEQ ID NO: 1254 - DNA, 21 bp, Homo sapiens
tattgagaaaggctggtctgc

SEQ ID NO: 1255 - RNA, 21 nt, RNAi-sense strand
uugagaaaggcuggucugcuu

SEQ ID NO: 1256 - RNA, 21 nt, RNAi-antisense strand
gcagaccagccuuucucaauu

SEQ ID NO: 1257 - DNA, 21 bp, Homo sapiens
taaagaacaagagtgtgatgt

SEQ ID NO: 1258 - RNA, 21 nt, RNAi-sense strand
aagaacaagagugugauguuu

SEQ ID NO: 1259 - RNA, 21 nt, RNAi-antisense strand
acaucacacucuuguucuuuu

SEQ ID NO: 1260 - DNA, 21 bp, Homo sapiens
tatcaacactccactgacaac

SEQ ID NO: 1261 - RNA, 21 nt, RNAi-sense strand
ucaacacuccacugacaacuu

SEQ ID NO: 1262 - RNA, 21 nt, RNAi-antisense strand
guugucaguggaguguugauu

SEQ ID NO: 1263 - DNA, 21 bp, Homo sapiens
tatctttcctcctgaaggaac

SEQ ID NO: 1264 - RNA, 21 nt, RNAi-sense strand
ucuuuccuccugaaggaacuu

SEQ ID NO: 1265 - RNA, 21 nt, RNAi-antisense strand
guuccuucaggaggaaagauu

SEQ ID NO: 1266 - DNA, 21 bp, Homo sapiens
tatatctgccaaggatattgt

SEQ ID NO: 1267 - RNA, 21 nt, RNAi-sense strand
uaucugccaaggauauuguuu

SEQ ID NO: 1268 - RNA, 21 nt, RNAi-antisense strand
acaauauccuuggcagauauu

SEQ ID NO: 1269 - DNA, 21 bp, Homo sapiens
tatctgccaaggatattgtgt

SEQ ID NO: 1270 - RNA, 21 nt, RNAi-sense strand
ucugccaaggauauuguguuu

SEQ ID NO: 1271 - RNA, 21 nt, RNAi-antisense strand
acacaauauccuuggcagauu

SEQ ID NO: 1272 - DNA, 21 bp, Homo sapiens
tattgtgtatattggcttgag

SEQ ID NO: 1273 - RNA, 21 nt, RNAi-sense strand
uuguguauauuggcuugaguu

SEQ ID NO: 1274 - RNA, 21 nt, RNAi-antisense strand
cucaagccaauauacacaauu

SEQ ID NO: 1275 - DNA, 21 bp, Homo sapiens
tatattggcttgagagacgtg

SEQ ID NO: 1276 - RNA, 21 nt, RNAi-sense strand
uauuggcuugagagacguguu

SEQ ID NO: 1277 - RNA, 21 nt, RNAi-antisense strand
cacgucucucaagccaauauu

SEQ ID NO: 1278 - DNA, 21 bp, Homo sapiens
tattggcttgagagacgtgga

SEQ ID NO: 1279 - RNA, 21 nt, RNAi-sense strand
uuggcuugagagacguggauu

SEQ ID NO: 1280 - RNA, 21 nt, RNAi-antisense strand
uccacgucucucaagccaauu

SEQ ID NO: 1281 - DNA, 21 bp, Homo sapiens
taggaattggcaaggtgatgg

SEQ ID NO: 1282 - RNA, 21 nt, RNAi-sense strand
ggaauuggcaaggugaugguu

SEQ ID NO: 1283 - RNA, 21 nt, RNAi-antisense strand
ccaucaccuugccaauuccuu

SEQ ID NO: 1284 - DNA, 21 bp, Homo sapiens
tacagagaaggtctctacatc

SEQ ID NO: 1285 - RNA, 21 nt, RNAi-sense strand
cagagaaggucucuacaucuu

SEQ ID NO: 1286 - RNA, 21 nt, RNAi-antisense strand
gauguagagaccuucucuguu

SEQ ID NO: 1287 - DNA, 21 bp, Homo sapiens
tacatcacagaagaaatctac

SEQ ID NO: 1288 - RNA, 21 nt, RNAi-sense strand
caucacagaagaaaucuacuu

SEQ ID NO: 1289 - RNA, 21 nt, RNAi-antisense strand
guagauuucuucugugauguu

SEQ ID NO: 1290 - DNA, 21 bp, Homo sapiens
tagatataatggaagtgaacc

SEQ ID NO: 1291 - RNA, 21 nt, RNAi-sense strand
gauauaauggaagugaaccuu

SEQ ID NO: 1292 - RNA, 21 nt, RNAi-antisense strand
gguucacuuccauuauaucuu

SEQ ID NO: 1293 - DNA, 21 bp, Homo sapiens
taactcgaacagtgaacacag

SEQ ID NO: 1294 - RNA, 21 nt, RNAi-sense strand
acucgaacagugaacacaguu

SEQ ID NO: 1295 - RNA, 21 nt, RNAi-antisense strand
cuguguucacuguucgaguuu

SEQ ID NO: 1296 - DNA, 21 bp, Homo sapiens
taaccttggcttgtttcggac

SEQ ID NO: 1297 - RNA, 21 nt, RNAi-sense strand
accuuggcuuguuucggacuu

SEQ ID NO: 1298 - RNA, 21 nt, RNAi-antisense strand
guccgaaacaagccaagguuu

SEQ ID NO: 1299 - DNA, 21 bp, Homo sapiens
taatcacaagcctattgacta

SEQ ID NO: 1300 - RNA, 21 nt, RNAi-sense strand
aucacaagccuauugacuauu

SEQ ID NO: 1301 - RNA, 21 nt, RNAi-antisense strand
uagucaauaggcuugugauuu

SEQ ID NO: 1302 - DNA, 21 bp, Homo sapiens
taagtaaatgtggaaacatcc

SEQ ID NO: 1303 - RNA, 21 nt, RNAi-sense strand
aguaaauguggaaacauccuu

SEQ ID NO: 1304 - RNA, 21 nt, RNAi-antisense strand
ggauguuuccacauuuacuuu

SEQ ID NO: 1305 - DNA, 21 bp, Homo sapiens
taaatgtggaaacatccgata

SEQ ID NO: 1306 - RNA, 21 nt, RNAi-sense strand
aauguggaaacauccgauauu

SEQ ID NO: 1307 - RNA, 21 nt, RNAi-antisense strand
uaucggauguuuccacauuuu

SEQ ID NO: 1308 - DNA, 21 bp, Homo sapiens
taagcatagagttatccttct

SEQ ID NO: 1309 - RNA, 21 nt, RNAi-sense strand
agcauagaguuauccuucuuu

SEQ ID NO: 1310 - RNA, 21 nt, RNAi-antisense strand
agaaggauaacucuaugcuuu

SEQ ID NO: 1311 - DNA, 21 bp, Homo sapiens
tagagttatccttctaaagac

SEQ ID NO: 1312 - RNA, 21 nt, RNAi-sense strand
gaguuauccuucuaaagacuu

SEQ ID NO: 1313 - RNA, 21 nt, RNAi-antisense strand
gucuuuagaaggauaacucuu

SEQ ID NO: 1314 - DNA, 21 bp, Homo sapiens
tatccttctaaagacttgttc

SEQ ID NO: 1315 - RNA, 21 nt, RNAi-sense strand
uccuucuaaagacuuguucuu

SEQ ID NO: 1316 - RNA, 21 nt, RNAi-antisense strand
gaacaagucuuuagaaggauu

SEQ ID NO: 1317 - DNA, 21 bp, Homo sapiens
tatccttagaaagagaagtgt

SEQ ID NO: 1318 - RNA, 21 nt, RNAi-sense strand
uccuuagaaagagaaguguuu

SEQ ID NO: 1319 - RNA, 21 nt, RNAi-antisense strand
acacuucucuuucuaaggauu

SEQ ID NO: 1320 - DNA, 21 bp, Homo sapiens
tagaaagagaagtgtacattg

SEQ ID NO: 1321 - RNA, 21 nt, RNAi-sense strand
gaaagagaaguguacauuguu

SEQ ID NO: 1322 - RNA, 21 nt, RNAi-antisense strand
caauguacacuucucuuucuu

SEQ ID NO: 1323 - DNA, 21 bp, Homo sapiens
taccacatgtggaaaggtact

SEQ ID NO: 1324 - RNA, 21 nt, RNAi-sense strand
ccacauguggaaagguacuuu

SEQ ID NO: 1325 - RNA, 21 nt, RNAi-antisense strand
aguaccuuuccacaugugguu

SEQ ID NO: 1326 - DNA, 21 bp, Homo sapiens
tactatgtgtccatgtcattc

SEQ ID NO: 1327 - RNA, 21 nt, RNAi-sense strand
cuauguguccaugucauucuu

SEQ ID NO: 1328 - RNA, 21 nt, RNAi-antisense strand
gaaugacauggacacauaguu

SEQ ID NO: 1329 - DNA, 21 bp, Homo sapiens
tatgtgtccatgtcattcaaa

SEQ ID NO: 1330 - RNA, 21 nt, RNAi-sense strand
uguguccaugucauucaaauu

SEQ ID NO: 1331 - RNA, 21 nt, RNAi-antisense strand
uuugaaugacauggacacauu

SEQ ID NO: 1332 - DNA, 21 bp, Homo sapiens
aagattagtgaagagtttctc

SEQ ID NO: 1333 - RNA, 21 nt, RNAi-sense strand
gauuagugaagaguuucucuu

SEQ ID NO: 1334 - RNA, 21 nt, RNAi-antisense strand
gagaaacucuucacuaaucuu

SEQ ID NO: 1335 - DNA, 21 bp, Homo sapiens
aattgtgacgtctttgcttgt

SEQ ID NO: 1336 - RNA, 21 nt, RNAi-sense strand
uugugacgucuuugcuuguuu

SEQ ID NO: 1337 - RNA, 21 nt, RNAi-antisense strand
acaagcaaagacgucacaauu

SEQ ID NO: 1338 - DNA, 21 bp, Homo sapiens
aacacttatgatgccgtacta

SEQ ID NO: 1339 - RNA, 21 nt, RNAi-sense strand
cacuuaugaugccguacuauu

SEQ ID NO: 1340 - RNA, 21 nt, RNAi-antisense strand
uaguacggcaucauaaguguu

SEQ ID NO: 1341 - DNA, 21 bp, Homo sapiens
aaatatgtcgtcgcagctggt

SEQ ID NO: 1342 - RNA, 21 nt, RNAi-sense strand
auaugucgucgcagcugguuu

SEQ ID NO: 1343 - RNA, 21 nt, RNAi-antisense strand
accagcugcgacgacauauuu

SEQ ID NO: 1344 - DNA, 21 bp, Homo sapiens
aatatgtcgtcgcagctggtt

SEQ ID NO: 1345 - RNA, 21 nt, RNAi-sense strand
uaugucgucgcagcugguuuu

SEQ ID NO: 1346 - RNA, 21 nt, RNAi-antisense strand
aaccagcugcgacgacauauu

SEQ ID NO: 1347 - DNA, 21 bp, Homo sapiens
aatgaagaagatgcttatcca

SEQ ID NO: 1348 - RNA, 21 nt, RNAi-sense strand
ugaagaagaugcuuauccauu

SEQ ID NO: 1349 - RNA, 21 nt, RNAi-antisense strand
uggauaagcaucuucuucauu

SEQ ID NO: 1350 - DNA, 21 bp, Homo sapiens
caaactggaagattagtgaag

SEQ ID NO: 1351 - RNA, 21 nt, RNAi-sense strand
aacuggaagauuagugaaguu

SEQ ID NO: 1352 - RNA, 21 nt, RNAi-antisense strand
cuucacuaaucuuccaguuuu

SEQ ID NO: 1353 - DNA, 21 bp, Homo sapiens
cattgcaacaactggtgaaga

SEQ ID NO: 1354 - RNA, 21 nt, RNAi-sense strand
uugcaacaacuggugaagauu

SEQ ID NO: 1355 - RNA, 21 nt, RNAi-antisense strand
ucuucaccaguuguugcaauu

SEQ ID NO: 1356 - DNA, 21 bp, Homo sapiens
cattggaattgtgacgtcttt

SEQ ID NO: 1357 - RNA, 21 nt, RNAi-sense strand
uuggaauugugacgucuuuuu

SEQ ID NO: 1358 - RNA, 21 nt, RNAi-antisense strand
aaagacgucacaauuccaauu

SEQ ID NO: 1359 - DNA, 21 bp, Homo sapiens
cagctttaacacttatgatgc

SEQ ID NO: 1360 - RNA, 21 nt, RNAi-sense strand
gcuuuaacacuuaugaugcuu

SEQ ID NO: 1361 - RNA, 21 nt, RNAi-antisense strand
gcaucauaaguguuaaagcuu

SEQ ID NO: 1362 - DNA, 21 bp, Homo sapiens
cacttatgatgccgtactacc

SEQ ID NO: 1363 - RNA, 21 nt, RNAi-sense strand
cuuaugaugccguacuaccuu

SEQ ID NO: 1364 - RNA, 21 nt, RNAi-antisense strand
gguaguacggcaucauaaguu

SEQ ID NO: 1365 - DNA, 21 bp, Homo sapiens
caaatatgtcgtcgcagctgg

SEQ ID NO: 1366 - RNA, 21 nt, RNAi-sense strand
aauaugucgucgcagcugguu

SEQ ID NO: 1367 - RNA, 21 nt, RNAi-antisense strand
ccagcugcgacgacauauuuu

SEQ ID NO: 1368 - DNA, 21 bp, Homo sapiens
cacactcatggcctactctct

SEQ ID NO: 1369 - RNA, 21 nt, RNAi-sense strand
cacucauggccuacucucuuu

SEQ ID NO: 1370 - RNA, 21 nt, RNAi-antisense strand
agagaguaggccaugaguguu

SEQ ID NO: 1371 - DNA, 21 bp, Homo sapiens
cagcctggcttatcttacgac

SEQ ID NO: 1372 - RNA, 21 nt, RNAi-sense strand
gccuggcuuaucuuacgacuu

SEQ ID NO: 1373 - RNA, 21 nt, RNAi-antisense strand
gucguaagauaagccaggcuu

SEQ ID NO: 1374 - DNA, 21 bp, Homo sapiens
cagcttctctcgtgagctttc

SEQ ID NO: 1375 - RNA, 21 nt, RNAi-sense strand
gcuucucucgugagcuuucuu

SEQ ID NO: 1376 - RNA, 21 nt, RNAi-antisense strand
gaaagcucacgagagaagcuu

SEQ ID NO: 1377 - DNA, 21 bp, Homo sapiens
cacttacggagttcatgccat

SEQ ID NO: 1378 - RNA, 21 nt, RNAi-sense strand
cuuacggaguucaugccauuu

SEQ ID NO: 1379 - RNA, 21 nt, RNAi-antisense strand
auggcaugaacuccguaaguu

SEQ ID NO: 1380 - DNA, 21 bp, Homo sapiens
catttacttgatggtccagtt

SEQ ID NO: 1381 - RNA, 21 nt, RNAi-sense strand
uuuacuugaugguccaguuuu

SEQ ID NO: 1382 - RNA, 21 nt, RNAi-antisense strand
aacuggaccaucaaguaaauu

SEQ ID NO: 1383 - DNA, 21 bp, Homo sapiens
cagacaacgttcatgcagcag

SEQ ID NO: 1384 - RNA, 21 nt, RNAi-sense strand
gacaacguucaugcagcaguu

SEQ ID NO: 1385 - RNA, 21 nt, RNAi-antisense strand
cugcugcaugaacguugucuu

SEQ ID NO: 1386 - DNA, 21 bp, Homo sapiens
gactgcattgcaacaactggt

SEQ ID NO: 1387 - RNA, 21 nt, RNAi-sense strand
cugcauugcaacaacugguuu

SEQ ID NO: 1388 - RNA, 21 nt, RNAi-antisense strand
accaguuguugcaaugcaguu

SEQ ID NO: 1389 - DNA, 21 bp, Homo sapiens
gaattgtgacgtctttgcttg

SEQ ID NO: 1390 - RNA, 21 nt, RNAi-sense strand
auugugacgucuuugcuuguu

SEQ ID NO: 1391 - RNA, 21 nt, RNAi-antisense strand
caagcaaagacgucacaauuu

SEQ ID NO: 1392 - DNA, 21 bp, Homo sapiens
gagtaagaggcagtcaccagt

SEQ ID NO: 1393 - RNA, 21 nt, RNAi-sense strand
guaagaggcagucaccaguuu

SEQ ID NO: 1394 - RNA, 21 nt, RNAi-antisense strand
acuggugacugccucuuacuu

SEQ ID NO: 1395 - DNA, 21 bp, Homo sapiens
gaaggcgcttgtggacatgat

SEQ ID NO: 1396 - RNA, 21 nt, RNAi-sense strand
aggcgcuuguggacaugauuu

SEQ ID NO: 1397 - RNA, 21 nt, RNAi-antisense strand
aucauguccacaagcgccuuu

SEQ ID NO: 1398 - DNA, 21 bp, Homo sapiens
gattcctagctttcctcgtgt

SEQ ID NO: 1399 - RNA, 21 nt, RNAi-sense strand
uuccuagcuuuccucguguuu

SEQ ID NO: 1400 - RNA, 21 nt, RNAi-antisense strand
acacgaggaaagcuaggaauu

SEQ ID NO: 1401 - DNA, 21 bp, Homo sapiens
taacacttatgatgccgtact

SEQ ID NO: 1402 - RNA, 21 nt, RNAi-sense strand
acacuuaugaugccguacuuu

SEQ ID NO: 1403 - RNA, 21 nt, RNAi-antisense strand
aguacggcaucauaaguguuu

SEQ ID NO: 1404 - DNA, 21 bp, Homo sapiens
tatgatgccgtactacctcct

SEQ ID NO: 1405 - RNA, 21 nt, RNAi-sense strand
ugaugccguacuaccuccuuu

SEQ ID NO: 1406 - RNA, 21 nt, RNAi-antisense strand
aggagguaguacggcaucauu

SEQ ID NO: 1407 - DNA, 21 bp, Homo sapiens
tatccagacaacgttcatgca

SEQ ID NO: 1408 - RNA, 21 nt, RNAi-sense strand
uccagacaacguucaugcauu

SEQ ID NO: 1409 - RNA, 21 nt, RNAi-antisense strand
ugcaugaacguugucuggauu

SEQ ID NO: 1410 - DNA, 21 bp, Homo sapiens
aagaacaagagtgtgatgtga

SEQ ID NO: 1411 - RNA, 21 nt, RNAi-sense strand
gaacaagagugugaugugauu

SEQ ID NO: 1412 - RNA, 21 nt, RNAi-antisense strand
ucacaucacacucuuguucuu

SEQ ID NO: 1413 - DNA, 21 bp, Homo sapiens
aacaagagtgtgatgtgaagg

SEQ ID NO: 1414 - RNA, 21 nt, RNAi-sense strand
caagagugugaugugaagguu

SEQ ID NO: 1415 - RNA, 21 nt, RNAi-antisense strand
ccuucacaucacacucuuguu

SEQ ID NO: 1416 - DNA, 21 bp, Homo sapiens
aagagtgtgatgtgaaggatt

SEQ ID NO: 1417 - RNA, 21 nt, RNAi-sense strand
gagugugaugugaaggauuuu

SEQ ID NO: 1418 - RNA, 21 nt, RNAi-antisense strand
aauccuucacaucacacucuu

SEQ ID NO: 1419 - DNA, 21 bp, Homo sapiens
aacactccactgacaaccaca

SEQ ID NO: 1420 - RNA, 21 nt, RNAi-sense strand
cacuccacugacaaccacauu

SEQ ID NO: 1421 - RNA, 21 nt, RNAi-antisense strand
ugugguugucaguggaguguu

SEQ ID NO: 1422 - DNA, 21 bp, Homo sapiens
aaacttgcatggacaacctgt

SEQ ID NO: 1423 - RNA, 21 nt, RNAi-sense strand
acuugcauggacaaccuguuu

SEQ ID NO: 1424 - RNA, 21 nt, RNAi-antisense strand
acagguuguccaugcaaguuu

SEQ ID NO: 1425 - DNA, 21 bp, Homo sapiens
aacttgcatggacaacctgta

SEQ ID NO: 1426 - RNA, 21 nt, RNAi-sense strand
cuugcauggacaaccuguauu

SEQ ID NO: 1427 - RNA, 21 nt, RNAi-antisense strand
uacagguuguccaugcaaguu

SEQ ID NO: 1428 - DNA, 21 bp, Homo sapiens
aagtggacagactaggaattg

SEQ ID NO: 1429 - RNA, 21 nt, RNAi-sense strand
guggacagacuaggaauuguu

SEQ ID NO: 1430 - RNA, 21 nt, RNAi-antisense strand
caauuccuagucuguccacuu

SEQ ID NO: 1431 - DNA, 21 bp, Homo sapiens
aaaggccaattcatctaagtt

SEQ ID NO: 1432 - RNA, 21 nt, RNAi-sense strand
aggccaauucaucuaaguuuu

SEQ ID NO: 1433 - RNA, 21 nt, RNAi-antisense strand
aacuuagaugaauuggccuuu

SEQ ID NO: 1434 - DNA, 21 bp, Homo sapiens
aaggccaattcatctaagttt

SEQ ID NO: 1435 - RNA, 21 nt, RNAi-sense strand
ggccaauucaucuaaguuuuu

SEQ ID NO: 1436 - RNA, 21 nt, RNAi-antisense strand
aaacuuagaugaauuggccuu

SEQ ID NO: 1437 - DNA, 21 bp, Homo sapiens
aagacaccagaagaagtaact

SEQ ID NO: 1438 - RNA, 21 nt, RNAi-sense strand
gacaccagaagaaguaacuuu

SEQ ID NO: 1439 - RNA, 21 nt, RNAi-antisense strand
aguuacuucuucuggugucuu

SEQ ID NO: 1440 - DNA, 21 bp, Homo sapiens
aagaagtaactcgaacagtga

SEQ ID NO: 1441 - RNA, 21 nt, RNAi-sense strand
gaaguaacucgaacagugauu

SEQ ID NO: 1442 - RNA, 21 nt, RNAi-antisense strand
ucacuguucgaguuacuucuu

SEQ ID NO: 1443 - DNA, 21 bp, Homo sapiens
aagtaactcgaacagtgaaca

SEQ ID NO: 1444 - RNA, 21 nt, RNAi-sense strand
guaacucgaacagugaacauu

SEQ ID NO: 1445 - RNA, 21 nt, RNAi-antisense strand
uguucacuguucgaguuacuu

SEQ ID NO: 1446 - DNA, 21 bp, Homo sapiens
caagagtgtgatgtgaaggat

SEQ ID NO: 1447 - RNA, 21 nt, RNAi-sense strand
agagugugaugugaaggauuu

SEQ ID NO: 1448 - RNA, 21 nt, RNAi-antisense strand
auccuucacaucacacucuuu

SEQ ID NO: 1449 - DNA, 21 bp, Homo sapiens
cactccactgacaaccacaag

SEQ ID NO: 1450 - RNA, 21 nt, RNAi-sense strand
cuccacugacaaccacaaguu

SEQ ID NO: 1451 - RNA, 21 nt, RNAi-antisense strand
cuugugguugucaguggaguu

SEQ ID NO: 1452 - DNA, 21 bp, Homo sapiens
cactgacaaccacaagtggaa

SEQ ID NO: 1453 - RNA, 21 nt, RNAi-sense strand
cugacaaccacaaguggaauu

SEQ ID NO: 1454 - RNA, 21 nt, RNAi-antisense strand
uuccacuugugguugucaguu

SEQ ID NO: 1455 - DNA, 21 bp, Homo sapiens
caagtggaaacttgcatggac

SEQ ID NO: 1456 - RNA, 21 nt, RNAi-sense strand
aguggaaacuugcauggacuu

SEQ ID NO: 1457 - RNA, 21 nt, RNAi-antisense strand
guccaugcaaguuuccacuuu

SEQ ID NO: 1458 - DNA, 21 bp, Homo sapiens
catggacaacctgtatctttc

SEQ ID NO: 1459 - RNA, 21 nt, RNAi-sense strand
uggacaaccuguaucuuucuu

SEQ ID NO: 1460 - RNA, 21 nt, RNAi-antisense strand
gaaagauacagguuguccauu

SEQ ID NO: 1461 - DNA, 21 bp, Homo sapiens
cagactaggaattggcaaggt

SEQ ID NO: 1462 - RNA, 21 nt, RNAi-sense strand
gacuaggaauuggcaagguuu

SEQ ID NO: 1463 - RNA, 21 nt, RNAi-antisense strand
accuugccaauuccuagucuu

SEQ ID NO: 1464 - DNA, 21 bp, Homo sapiens
cactcagctatctactaggaa

SEQ ID NO: 1465 - RNA, 21 nt, RNAi-sense strand
cucagcuaucuacuaggaauu

SEQ ID NO: 1466 - RNA, 21 nt, RNAi-antisense strand
uuccuaguagauagcugaguu

SEQ ID NO: 1467 - DNA, 21 bp, Homo sapiens
cagctatctactaggaagaaa

SEQ ID NO: 1468 - RNA, 21 nt, RNAi-sense strand
gcuaucuacuaggaagaaauu

SEQ ID NO: 1469 - RNA, 21 nt, RNAi-antisense strand
uuucuuccuaguagauagcuu

SEQ ID NO: 1470 - DNA, 21 bp, Homo sapiens
cagagaaggtctctacatcac

SEQ ID NO: 1471 - RNA, 21 nt, RNAi-sense strand
gagaaggucucuacaucacuu

SEQ ID NO: 1472 - RNA, 21 nt, RNAi-antisense strand
gugauguagagaccuucucuu

SEQ ID NO: 1473 - DNA, 21 bp, Homo sapiens
gaacaagagtgtgatgtgaag

SEQ ID NO: 1474 - RNA, 21 nt, RNAi-sense strand
acaagagugugaugugaaguu

SEQ ID NO: 1475 - RNA, 21 nt, RNAi-antisense strand
cuucacaucacacucuuguuu

SEQ ID NO: 1476 - DNA, 21 bp, Homo sapiens
gagtgtgatgtgaaggattat

SEQ ID NO: 1477 - RNA, 21 nt, RNAi-sense strand
gugugaugugaaggauuauuu

SEQ ID NO: 1478 - RNA, 21 nt, RNAi-antisense strand
auaauccuucacaucacacuu

SEQ ID NO: 1479 - DNA, 21 bp, Homo sapiens
gaagaacggaagaatcagcct

SEQ ID NO: 1480 - RNA, 21 nt, RNAi-sense strand
agaacggaagaaucagccuuu

SEQ ID NO: 1481 - RNA, 21 nt, RNAi-antisense strand
aggcugauucuuccguucuuu

SEQ ID NO: 1482 - DNA, 21 bp, Homo sapiens
gagaccacagtttggcaattg

SEQ ID NO: 1483 - RNA, 21 nt, RNAi-sense strand
gaccacaguuuggcaauuguu

SEQ ID NO: 1484 - RNA, 21 nt, RNAi-antisense strand
caauugccaaacuguggucuu

SEQ ID NO: 1485 - DNA, 21 bp, Homo sapiens
gaccacagtttggcaattgga

SEQ ID NO: 1486 - RNA, 21 nt, RNAi-sense strand
ccacaguuuggcaauuggauu

SEQ ID NO: 1487 - RNA, 21 nt, RNAi-antisense strand
uccaauugccaaacugugguu

SEQ ID NO: 1488 - DNA, 21 bp, Homo sapiens
gatgctcacactgatatcaac

SEQ ID NO: 1489 - RNA, 21 nt, RNAi-sense strand
ugcucacacugauaucaacuu

SEQ ID NO: 1490 - RNA, 21 nt, RNAi-antisense strand
guugauaucagugugagcauu

SEQ ID NO: 1491 - DNA, 21 bp, Homo sapiens
gaaacttgcatggacaacctg

SEQ ID NO: 1492 - RNA, 21 nt, RNAi-sense strand
aacuugcauggacaaccuguu

SEQ ID NO: 1493 - RNA, 21 nt, RNAi-antisense strand
cagguuguccaugcaaguuuu

SEQ ID NO: 1494 - DNA, 21 bp, Homo sapiens
gaagtggacagactaggaatt

SEQ ID NO: 1495 - RNA, 21 nt, RNAi-sense strand
aguggacagacuaggaauuuu

SEQ ID NO: 1496 - RNA, 21 nt, RNAi-antisense strand
aauuccuagucuguccacuuu

SEQ ID NO: 1497 - DNA, 21 bp, Homo sapiens
gacagactaggaattggcaag

SEQ ID NO: 1498 - RNA, 21 nt, RNAi-sense strand
cagacuaggaauuggcaaguu

SEQ ID NO: 1499 - RNA, 21 nt, RNAi-antisense strand
cuugccaauuccuagucuguu

SEQ ID NO: 1500 - DNA, 21 bp, Homo sapiens
gaagaagtaactcgaacagtg

SEQ ID NO: 1501 - RNA, 21 nt, RNAi-sense strand
agaaguaacucgaacaguguu

SEQ ID NO: 1502 - RNA, 21 nt, RNAi-antisense strand
cacuguucgaguuacuucuuu

SEQ ID NO: 1503 - DNA, 21 bp, Homo sapiens
gaagtaactcgaacagtgaac

SEQ ID NO: 1504 - RNA, 21 nt, RNAi-sense strand
aguaacucgaacagugaacuu

SEQ ID NO: 1505 - RNA, 21 nt, RNAi-antisense strand
guucacuguucgaguuacuuu

SEQ ID NO: 1506 - DNA, 21 bp, Homo sapiens
taaagaacaagagtgtgatgt

SEQ ID NO: 1507 - RNA, 21 nt, RNAi-sense strand
aagaacaagagugugauguuu

SEQ ID NO: 1508 - RNA, 21 nt, RNAi-antisense strand
acaucacacucuuguucuuuu

SEQ ID NO: 1509 - DNA, 21 bp, Homo sapiens
tatatctgccaaggatattgt

SEQ ID NO: 1510 - RNA, 21 nt, RNAi-sense strand
uaucugccaaggauauuguuu

SEQ ID NO: 1511 - RNA, 21 nt, RNAi-antisense strand
acaauauccuuggcagauauu

SEQ ID NO: 1512 - DNA, 21 bp, Homo sapiens
tatctgccaaggatattgtgt

SEQ ID NO: 1513 - RNA, 21 nt, RNAi-sense strand
ucugccaaggauauuguguuu

SEQ ID NO: 1514 - RNA, 21 nt, RNAi-antisense strand
acacaauauccuuggcagauu

SEQ ID NO: 1515 - DNA, 21 bp, Homo sapiens
tattgtgtatattggcttgag

SEQ ID NO: 1516 - RNA, 21 nt, RNAi-sense strand
uuguguauauuggcuugaguu

SEQ ID NO: 1517 - RNA, 21 nt, RNAi-antisense strand
cucaagccaauauacacaauu

SEQ ID NO: 1518 - DNA, 18 bp, Mus musculus
cggtggttctggccatct

SEQ ID NO: 1519 - DNA, 31 bp, Mus musculus
ttacggtaccagccagaacaacctaatctgg

SEQ ID NO: 1520 - DNA, 19 bp, Mus musculus
tggctgcctgtgttttggt

SEQ ID NO: 1521 - DNA, 21 bp, Mus musculus
tcccaatgcctcgtgtaatct

SEQ ID NO: 1522 - DNA, 20 bp, Mus musculus
acccatcctccgccatagca

SEQ ID NO: 1523 - DNA, 26 bp, Mus musculus
tgatttgagctagacatttgaaaagc

SEQ ID NO: 1524 - DNA, 26 bp, Mus musculus
tggcttactgtttagatttcttgcaa

SEQ ID NO: 1525 - DNA, 24 bp, Mus musculus
agcaacgggtgactgcctcttact

SEQ ID NO: 1526 - DNA, 19 bp, Mus musculus
tgacccctgcagtcatcgt

SEQ ID NO: 1527 - DNA, 18 bp, Mus musculus
tgccttggcccttgtgtt

SEQ ID NO: 1528 - DNA, 21 bp, Mus musculus
aaaccagcaatccagtgagca

SEQ ID NO: 1529 - DNA, 24 bp, Mus musculus
ttggtaaccagggctgatttactt

SEQ ID NO: 1530 - DNA, 15 bp, Mus musculus
tccctgccgcgcatt

SEQ ID NO: 1531 - DNA, 18 bp, Mus musculus
agcccatcggcagccatg

SEQ ID NO: 1532 - DNA, 18 bp, Mus musculus
acgggcaaacacctggaa

SEQ ID NO: 1533 - DNA, 21 bp, Mus musculus
gtctggcagttggaagcatct

SEQ ID NO: 1534 - DNA, 18 bp, Mus musculus
tggcacgccagggtccac

SEQ ID NO: 1535 - DNA, 20 bp, Mus musculus
gcatccacccaaatgacaca